(19) United States
(12) Patent Application Publication
(10) Pub. No.: US 2005/0004599 A1
(43) Pub. Date: Jan. 6, 2005

(54) Non-light activated adhesive composite, system, and methods of use thereof

(76) Inventors: Karen M. McNally-Heintzelman, Cambridge, MA (US); Douglas L. Heintzelman, Cambridge, MA (US); Jeffrey N. Bloom, Chicago, IL (US); Mark T. Duffy, Chicago, IL (US)

Correspondence Address: BOSE MCKINNEY & EVANS LLP, 135 N PENNSYLVANIA ST, SUITE 2700, INDIANAPOLIS, IN, 46204, US

(21) Appl. No.: 10/610,068

(22) Filed: Jun. 30, 2003

(51) Int. Cl. A61D 1/00; A61B 17/08

(52) U.S. Cl. 606/214; 606/213

(57) ABSTRACT

The present invention provides a non-light activated adhesive composite, method, and system suitable for medical and surgical applications. The composite includes a scaffold and a non-light activated adhesive. The scaffold and the non-light activated adhesive include biological, biocompatible, or biodegradable materials.

### TECHNICAL FIELD

The present invention relates to the field of biological tissue repair and/or wound closure, e.g., after injury to the tissue or surgery. More particularly, the present invention relates to the use of biological or biocompatible adhesive composites for the repair of biological tissue.

### BACKGROUND

Known methods of biological tissue repair include sutures, staples and clips, sealants, and adhesives. Sutures are inexpensive, reliable, readily available and can be used on many types of lacerations and incisions. However, the use of sutures has many drawbacks. Sutures are intrusive in that they require puncturing of the tissue. Also, sutures require technical skill for their application, they can result in uneven healing, and they often necessitate patient follow-up visits for their removal. In addition, placement and removal of sutures in children may require sedation or anesthesia.

Staples or clips are preferred over sutures, for example, in minimally invasive endoscopic applications. Staples and clips require less time to apply than sutures, are available in different materials to suit different applications, and generally achieve uniform results. However, staples and clips are not easily adapted to different tissue dimensions and maintaining precision of alignment of the tissue is difficult because of the relatively large force required for application. Further, none of these fasteners is capable of producing a watertight seal for the repair.

Sealants, including fibrin-, collagen-, synthetic polymer- and protein-based sealants, act as a physical barrier to fluid and air, and can be used to promote wound healing, tissue regeneration and clot formation. However, sealants are generally time-consuming to prepare and apply. Also, with fibrin-based sealants, there is a risk of blood-borne viral disease transmission. Further, sealants cannot be used in high-tension areas.

Adhesives, for example, cyanoacrylate glues, have the advantage that they are generally easy to dispense. However, application of adhesives during the procedure can be cumbersome. Because of their liquid nature, these adhesives are difficult to precisely position on tissue and thus require adept and delicate application if precise positioning is desired. Cyanoacrylates also harden rapidly; therefore, the time available to the surgeon for proper tissue alignment is limited. Further, when cyanoacrylates dry, they become brittle. Thus, they cannot be used in areas of the body that have frequent movement. In addition, the currently available adhesives are not optimal for high-tension areas.

Laser tissue solders, or “light-activated adhesives,” are a possible alternative for overcoming the problems associated with the above-mentioned techniques. Laser tissue soldering is a bonding technique in which a protein solder is applied to the surface of the tissue(s) to be joined and laser energy is used to bond the solder to the tissue surface(s).

The use of biodegradable polymer scaffolding in laser-solder tissue repairs has been shown to improve the success rate and consistency of such repairs. See, for example, McNally et al., U.S. Pat. No. 6,391,049. However, a drawback of laser-soldering techniques is the need to supply light energy to the repair site to activate the adhesive. As a result, such techniques are only suitable for a limited number of clinical applications. For example, such techniques are generally not suitable for use outside of a hospital or other laser-equipped setting. Also, with laser techniques, there is always a risk of collateral thermal damage to the surrounding tissue.

Accordingly, there is a need for an improved method of biological tissue repair; particularly, a device or surgical product, system, and/or method which is capable of replacing the conventional suture, staple and clip techniques in a wide variety of applications.

### SUMMARY

A novel biocompatible or biological adhesive composite that results from the combination of a non-light activated adhesive and a scaffold material has been invented. This composite has exhibited surprisingly good tensile strength and consistency when compared with sutures and the use of adhesives alone. It can be used effectively as an adhesive, sealing or repairing device for biological tissue. It may also be used as a depot for drugs in providing medication to a wound or repair site. The composite can be precisely positioned across, on top of, or between two materials to be joined (i.e. tissue-to-tissue or tissue-to-biocompatible implant). Proper alignment is accomplished within the time period before the adhesive sets or hardens. Thus, the composite can be applied to a repair site more quickly and easily than sutures or adhesives alone. In addition, application of the composite can provide a watertight seal at the repair site when required.

The improved ease of clinical application makes the composite of the present invention applicable to all internal and external fields of surgery, extending from emergency neurosurgical and trauma procedures to elective cosmetic surgery, as well as to ophthalmic applications. Examples of external or topical applications for the composite include, but are not limited to, wound closure from trauma or at surgical incision sites. Internal surgical applications include, but are not limited to, repair of liver, spleen, or pancreas lacerations from trauma, dural laceration/incision closure, pneumothorax repair during thoracotomy, sealing points of vascular access following endovascular procedures, vascular anastomoses, tympanoplasty, endoscopic treatment of gastrointestinal ulcers/bleeds, dental applications for mucosal ulcerations or splinting of injured teeth, ophthalmologic surgeries, tendon and ligament repair in orthopedics, episiotomy/vaginal tear repair in gynecology. Additionally, as minimally invasive techniques become more common, the application of this technology to endoscopic, laparoscopic or endovascular techniques is very promising. With appropriate single-use packaging, the invention offers the potential for quick application in the field by less skilled professionals, paraprofessionals and bystanders in emergency situations—both military and civilian—outside a hospital or clinic setting.

Various techniques for forming the composite of the present invention and/or applying it to a wound or tissue repair site may be used. Additionally, there are numerous suitable alternatives for packaging the composite depending on the desired use, environment, or applications.

In accordance with the present invention, a composition suitable for medical and surgical applications is provided. The composition includes a scaffold including at least one of a biological material, biocompatible material, and biodegradable material, and a non-light activated adhesive including at least one of a biological material, biocompatible material, and biodegradable material. The non-light activated adhesive is combined with the scaffold to form a composite that, when used to repair biological tissue, has a tensile strength of at least about 120% of the tensile strength of the adhesive alone.

Also in accordance with the present invention, a method for repairing, joining, aligning, or sealing biological tissue is provided. The method includes the steps of combining a biological, biocompatible, or biodegradable scaffold and a non-light activated biological, biocompatible, or biodegradable adhesive to form a composite having a tensile strength of at least about 120% of the tensile strength of the adhesive alone, and applying the composite to an adhesion site.

Yet further in accordance with the present invention, a product for joining, repairing, aligning or sealing biological tissue is provided. The product includes a biological, biocompatible, or biodegradable scaffold, a biological, biocompatible, or biodegradable non-light activated adhesive, and means for coupling the scaffold and the adhesive to form a composite having a tensile strength of at least about 120% of the tensile strength of the adhesive alone.

### BRIEF DESCRIPTION OF THE DRAWINGS

FIG. 1 is a graph summarizing results obtained during the studies described in Example 1, comparing the maximum strength of repairs formed in organ specimens quoted as a percentage of native tissue strength;

FIG. 2 is a graph summarizing results obtained during the studies described in Example 1, comparing the maximum strength of repairs formed in vascular specimens quoted as a percentage of native tissue strength;

FIGS. 3A-3B are photographs showing the surgical technique used in Example 2 to perform strabismus surgery on rabbit eyes using cyanoacrylate glue alone;

FIG. 4A-4C are photographs showing the surgical technique used in Example 2 to perform strabismus surgery on rabbit eyes using scaffold-enhanced cyanoacrylate glue;

FIG. 5 is a photograph of the incision sites on the dorsal skin of a rat taken immediately following the repair of each incision using one of the four techniques described in Example 3;

FIG. 6 is a graph summarizing results obtained during the studies described in Example 3, showing the tensile strength of skin repairs performed using four different repair techniques seven days postoperatively;

FIG. 7 is a graph summarizing results obtained during the studies described in Example 3, showing the time to failure of the skin repairs seven days postoperatively;

FIG. 8A is a low magnification photomicrograph from Example 3 of rat skin 7 days after standardized full-thickness incision and repair with a 5-0 Nylon suture. E=keratinized squamous epithelium; D=dermis; ST=suture & suture tract; M=subdermal muscular layer; *=granulation tissue and healed wound tract;

FIG. 8B is a low magnification photomicrograph from Example 3 of rat skin 7 days after standardized full-thickness incision and repair by standard external application of cyanoacrylate (Dermabond™). E=keratinized squamous epithelium; D=dermis; SIR=superficial inflammatory reaction; M=subdermal muscular layer; *=granulation tissue and healed wound tract;

FIG. 8C is a low magnification photomicrograph from Example 3 of rat skin 7 days after standardized full-thickness incision and repair by external application of PLGA scaffold combined with cyanoacrylate. E=keratinized squamous epithelium; D=dermis; SIR=superficial inflammatory reaction; M=subdermal muscular layer; *=granulation tissue and healed wound tract; BV=blood vessel;

FIG. 9 is a graph summarizing results obtained during the studies described in Example 3, showing the tensile strength of skin repairs performed using four different repair techniques fourteen days postoperatively;

FIG. 10 is a graph summarizing results obtained during the studies described in Example 3, showing the time to failure of the skin repairs fourteen days postoperatively;

FIG. 11 is a graph summarizing tensile strength data from the studies described in Example 4;

FIG. 12 is a graph comparing time of failure for repairs tested in the studies described in Example 4;

FIG. 13A is an electron micrograph (magnification: 120×) of the smooth (intimal) surface of SIS used in studies described in Example 5;

FIG. 13B is an electron micrograph (magnification: 120×) of the irregular surface of SIS used in studies described in Example 5;

FIG. 14A is an electron micrograph (magnification: 120×) of the smooth (intimal) surface of PLGA used in studies described in Example 5;

FIG. 14B is an electron micrograph (magnification: 120×) of the irregular surface of PLGA used in studies described in Example 5;

FIG. 15 is a graph summarizing tensile strength results from the studies described in Example 5;

FIG. 16 is a graph summarizing time to failure results from the studies described in Example 5;

FIG. 17 is a graph summarizing tensile strength results from the studies described in Example 6;

FIG. 18 is a graph summarizing time to failure results from the studies described in Example 6;

FIGS. 19A-19D are electron micrographs (magnification: 120×) of irregularities added to the scaffold in studies described in Example 7;

FIG. 20 is a graph summarizing tensile strength results from the studies described in Example 7;

FIG. 21 is a graph summarizing time to failure results from the studies described in Example 7;

FIGS. 22A-22G are photographs of example embodiments of the disclosed scaffold;

FIG. 23 is a schematic representation of example embodiments of the disclosed scaffold;

FIGS. 24A and 24B are schematic representations of one embodiment of a form of packaging the composite, showing the scaffold isolated from the adhesive until the composite is needed for application to a wound or repair site;

FIG. 25 is another embodiment of a form of packaging the composite, showing the scaffold isolated from the adhesive until the composite is needed for application to a wound or repair site; and

FIGS. 26A and 26B are an illustrated representation of an application of one embodiment of the composite, showing how the scaffold provides biologically active materials to the tissue.

### DETAILED DESCRIPTION

Several experimental studies have confirmed the effectiveness of the present composite, which comprises a non-light activated adhesive and a scaffold, for biological tissue repair. The attached Appendix, incorporated herein by this reference, includes data tables relating to these studies. While specific compounds have been used in these studies, it is understood that the composite of the present invention is not limited to the particular compounds used in any of the disclosed examples.

The scaffold and adhesive used to form the composite of the present invention may each be composed of either biologic or synthetic materials. Examples of biologic materials that may be used as adhesives include, but are not limited to, serum albumin, collagen, fibrin, fibrinogen, fibronectin, thrombin, barnacle glues and marine algae. Examples of synthetic materials suitable for use as adhesives include, but are not limited to, cyanoacrylate (e.g., ethyl-, propyl-, butyl- and octyl-) glues. The biologic materials are, by their very nature, biodegradable. Currently marketed synthetic adhesives such as cyanoacrylates are not in themselves biodegradable, but processes can be applied to make them biodegradable. For example, a formaldehyde-scavenging process can be applied that allows the product to degrade in the body without producing a toxic reaction.

The mechanism by which the adhesive material bonds to the tissue, and thus, the determination of whether any auxiliary equipment is necessary, is dependent at least in part on the selection of the adhesive material. Some non-light activated adhesives require an activator or initiator (other than laser energy) to cause or accelerate bonding. For example, polymerization of octyl-cyanoacrylates can be accelerated through contact with a chemical initiator such as that contained in the tip of the applicator of Ethicon's Dermabond™. Cohesion's CoStasis and Cryolife's Bioglue also rely on the addition of an activator at the time of application, namely, fibrinogen and glutaraldehyde, respectively. It is understood that all of the above-mentioned adhesives, whether or not they require an initiator or activator, are considered “non-light activated” adhesives.

The scaffold operates to ensure continuous, consistent alignment of the apposed tissue edges. The scaffold also helps ensure that the tensile strength of the apposed edges is sufficient for healing to occur without the use of sutures, staples, clips, or other mechanical closures or means of reinforcement. By keeping the tissue edges in direct apposition, the scaffold helps foster primary intention healing and direct re-apposition internally. Thus, the scaffold functions as a bridge or framework for the apposed edges of severed tissue.

As mentioned above, the scaffold is either a synthetic or biological material. A suitable biological scaffold comprises SIS (small intestine submucosa), polymerized collagen, polymerized elastin, or other similarly suitable biological materials. Examples of synthetic materials suitable for use as a scaffold include, but are not limited to, various poly(alpha ester)s such as poly(lactic acid) (PLA), poly(glycolic acid) (PGA), poly(L-lactic-co-glycolic acid) (PLGA), poly(.epsilon.-caprolactone) (PGA) and poly(ethylene glycol) (PEG), as well as poly(alpha ester)s, poly(ortho ester)s and poly(anhydrides).

In alternative embodiments, the scaffold is engineered for specific applications of the composite by adjusting one or more of its properties. For example, the scaffold includes a smooth surface. Alternatively or in addition, the scaffold includes an irregular surface. Key properties of the scaffold are surface regularity or irregularity, elasticity, strength, porosity, surface area, degradation rate, and flexibility.

For purposes of this disclosure, “irregular” means that at least a portion of a surface of the scaffold is discontinuous or uneven, whether due to inherent porosity, roughness or other irregularities, or as a result of custom-engineering performed to introduce irregularities or roughness onto the surface (for example, using drilling, punching, or molding manufacturing techniques).

In further embodiments of the present invention, the scaffold is engineered to allow it to function as a depot for various dopants or biologically-active materials, such as antibiotics, anesthetics, anti-inflammatories, bacteriostatic or bacteriocidals, chemotherapeutic agents, vitamins, anti- or pro- neovascular or tissue cell growth factors, hemostatic and thrombogenic agents. This is accomplished by altering the macromolecular structure of the scaffold in order to adjust, for example, its porosity and/or degradation rate.

### EXAMPLE 1

### Comparison of Scaffold-Enhanced Albumin and n-Butyl-Cyanoacrylate Adhesives for Joining of Tissue in a Porcine Model

An ex vivo study was conducted to compare the tensile strength of tissue samples repaired using three different techniques: (i) application of a scaffold-enhanced light-activated albumin protein solder (Group I), (ii) application of a scaffold-enhanced n-butyl-cyanoacrylate (non-light activated) adhesive composite (Group II), and (iii) repair via conventional suture technique (Group III).

1.1 Preparation of the Surgical Adhesive

Porous synthetic polymer scaffolds were prepared from poly(L-lactic-co-glycolic acid) (PLGA), with a lactic:glycolic acid ratio of 85:15, using a solvent-casting and particulate leaching technique. The scaffolds were cast by dissolving 200 mg PLGA (Sigma Chemical Company, St. Louis, Mo.) in 2 mL dichloromethane (Sigma Chemical Company). Sodium chloride (salt particle size: 106-150 nm) with a 70% weight fraction was added to the polymer mix. The polymer solution was then spread to cover the bottom surface of a 60 mm diameter Petri dish that was cleaned first with dichloromethane, then ethanol, then ultra-filtered deionized water (Fisher Scientific, Pittsburgh, Pa.). The polymer was left in a fume hood for 24 hours to allow the dichloromethane to evaporate. The salt was leached out of the polymer scaffolds by immersion in filtered deionized water for 24 hours, to create the porous scaffolds. During this period the water was changed 3-4 times. The scaffolds were then air dried and stored at room temperature until required.

The PLGA scaffolds used for incision repair were cut into rectangular pieces with dimensions of 12±2 mm long by 5±1 mm wide. The scaffolds used for Group I were left to soak for a minimum of two hours before use in a protein solder mix comprised of 50% (w/v) bovine serum albumin (BSA) (Cohn Fraction V, Sigma Chemical Company) and Indocyanine Green (ICG) dye (Sigma Chemical Company) at a concentration of 0.5 mg/mL, mixed in deionized water. The thickness of the resulting scaffold-enhanced solders, determined by scanning electron microscopy and measurement with precision calipers (L. S. Starrett Co., Anthol, Mass.), was in the range of 200 to 205 μm. N-butyl-cyanoacrylate (Vetbond, 3M) was applied to the scaffolds used for Group II using a 22-G syringe immediately prior to application to the tissue.

1.2 Tissue Preparation

Porcine tissue specimens were harvested approximately 30 minutes after sacrificing the animals. Tissue specimens were stored in phosphate buffered saline for a maximum of two hours before they were prepared for experiments. Each tissue specimen was cut into small rectangular pieces with dimensions of about 2 cm long by 1 cm wide and a thickness of approximately 1.5±0.5 mm. Tissue specimens harvested included the small intestine, spleen, muscle, skin, atrium, ventricle, lung, pancreas, liver, gall bladder, kidney, ureter, sciatic nerve, carotid artery, femoral artery, splenic artery, coronary artery, pulmonary artery and aorta (both intima and adventitia). Ten repairs were performed for each tissue type and repair procedure investigated.

1.3 Incision Repair

A full thickness incision was cut through each specimen width using a scalpel, and opposing ends were placed together. All laser-assisted repairs were completed with a diode laser operating at a wavelength of 808-nm (Spectra Physics, Mountain View, Ca.). The laser light was coupled into a 660-μm diameter silica fiber bundle and focused onto the scaffold surface with an imaging hand-piece connected at the end of the fiber. The diode was operated in continuous mode with a spot size of approximately 1 mm at the surface of the scaffold-enhanced solder. An aiming beam was also incorporated into the system and was delivered through the same fiber as the 808-nm beam. The laser beam was scanned across the scaffold-enhanced solder twice, starting from the center and moving outwards in a spiral pattern with a total irradiation time of 80±2 seconds. Suture repairs were achieved using a single 4-0 nylon suture.

1.4 Strength Testing

Tensile strength measurements were performed to test the integrity of the resultant repairs immediately following the laser procedure using a calibrated MTS Material Strength Testing Machine (858 Table Top System, MTS, Eden Prairie, Minn.). This system was interfaced with a personal computer to collect the data. Each tissue specimen was clamped to the strength testing machine using a 100N load cell with pneumatic grips. The specimens were pulled apart at a rate of 100 gf/min until the repair failed. Complete separation at the tissue edges defined failure. The maximum load in Newton's was recorded at the breaking point. The strengths of corresponding native specimens were tested and used as references. Native tissue specimens were prepared for tensile testing in an identical manner to the experimental repair group specimens, with the exception that microscissors were used to cut in from each edge with care to leave a 5±1 mm bridge of tissue in the center. This spacing matched the width of the scaffold-enhanced adhesives used on specimens from Groups I and II.

1.5 Results

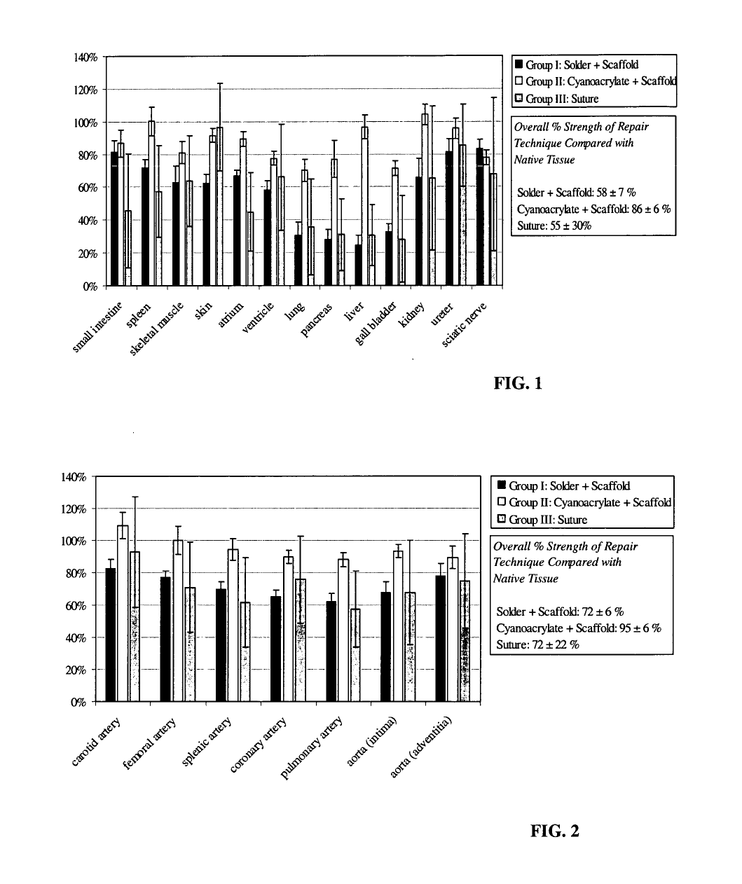

The tensile strengths recorded at the breaking point of the repaired organ specimens are recorded in Table 1 and displayed in FIG. 1. Table 2 and FIG. 2 list and display the tensile strengths recorded at the breaking point for the repaired vessel specimens. Tables A and B of the Appendix include more detailed data relating to Example 1. All measurements in FIGS. 1 and 2 are quoted as the percent strength of native tissue. In Group I and II, all repairs failed interfacially (at the solder/tissue interface), that is, the adhesive remained intact but detached from the tissue. In Group III, all repairs failed with the suture pulling through the tissue specimen.

Group I repairs formed on the ureter were the most successful followed by the small intestine, sciatic nerve, spleen, atrium, kidney, muscle, skin and ventricle. The repairs on the ureter, small intestine and sciatic nerve achieved 81-83% of the strength of native tissue while repairs on the spleen, atrium and kidney attained approximately 66-72% of the strength of native tissue. Group I repairs performed on the liver, pancreas, lung and gallbladder specimens resulted in a very weak bond between the scaffold-enhanced solder and tissue, at only approximately 24-33% of the strength of native specimens. The strongest Group I vascular repairs were achieved in the carotid arteries, aorta (adventitia) and femoral arteries where breaking strengths of approximately 83%, 78% and 77% of their native tissue specimens, respectively, were achieved.

Although, the weakest vascular repairs were made on the pulmonary artery, the repairs still achieved greater than 62% of the strength of the native tissue. The overall percentage repair strength of native tissue was equivalent between Groups I and III (Group I Organs: 58±21%; Group III Organs: 55±22%; Group I Vessels: 72±8%; Group III Vessels: 72±12%). This does not imply, however, that the strength of Group I and Group III repairs were equivalent for each tissue type (see FIGS. 1 and 2).

TABLE 1

| | Tensile Strength (N) | | | |
|---|---|---|---|---|
| | Solder + | Cyanoacrylate + | Single 4-0 | |
| Tissue | Scaffold | Scaffold | Suture | Native Tissue |
| small intestine | 0.87 ± 0.08 | 0.93 ± 0.09 | 0.49 ± 0.37 | 1.07 ± 0.09 |
| spleen | 0.65 ± 0.05 | 0.90 ± 0.08 | 0.52 ± 0.25 | 0.90 ± 0.05 |
| skeletal muscle | 1.20 ± 0.19 | 1.54 ± 0.13 | 1.21 ± 0.53 | 1.90 ± 0.08 |
| skin | 1.02 ± 0.10 | 1.50 ± 0.07 | 1.58 ± 0.44 | 1.64 ± 0.07 |
| atrium | 0.89 ± 0.05 | 1.20 ± 0.06 | 0.60 ± 0.32 | 1.34 ± 0.05 |
| ventricle | 0.83 ± 0.08 | 1.10 ± 0.06 | 0.94 ± 0.46 | 1.42 ± 0.07 |
| lung | 0.22 ± 0.06 | 0.50 ± 0.05 | 0.25 ± 0.21 | 0.72 ± 0.06 |
| pancreas | 0.36 ± 0.08 | 0.99 ± 0.15 | 0.40 ± 0.28 | 1.29 ± 0.06 |
| liver | 0.34 ± 0.09 | 1.32 ± 0.10 | 0.42 ± 0.26 | 1.37 ± 0.06 |
| gall bladder | 0.42 ± 0.06 | 0.92 ± 0.06 | 0.37 ± 0.34 | 1.29 ± 0.08 |
| kidney | 0.61 ± 0.11 | 0.97 ± 0.06 | 0.61 ± 0.41 | 0.93 ± 0.13 |
| ureter | 1.01 ± 0.10 | 1.18 ± 0.08 | 1.05 ± 0.31 | 1.23 ± 0.07 |
| sciatic nerve | 0.91 ± 0.06 | 0.85 ± 0.05 | 0.74 ± 0.51 | 1.10 ± 0.07 |

TABLE 2

| | Tensile Strength (N) | | | |
|---|---|---|---|---|
| | Solder + | Cyanoacrylate + | Single 4-0 | |
| Tissue | Scaffold | Scaffold | Suture | Native Tissue |
| carotid artery | 0.76 ± 0.05 | 1.01 ± 0.08 | 0.85 ± 0.34 | 0.92 ± 0.05 |
| femoral artery | 0.79 ± 0.04 | 1.02 ± 0.04 | 0.72 ± 0.24 | 1.02 ± 0.04 |
| splenic artery | 1.04 ± 0.07 | 1.40 ± 0.08 | 0.92 ± 0.42 | 1.49 ± 0.04 |
| coronary artery | 1.01 ± 0.07 | 1.39 ± 0.07 | 1.17 ± 0.25 | 1.55 ± 0.05 |
| pulmonary artery | 0.94 ± 0.08 | 1.34 ± 0.10 | 0.87 ± 0.23 | 1.52 ± 0.07 |
| aorta (intima) | 1.08 ± 0.11 | 1.48 ± 0.11 | 1.07 ± 0.39 | 1.59 ± 0.05 |
| aorta (adventitia) | 1.24 ± 0.12 | 1.42 ± 0.06 | 1.19 ± 0.19 | 1.59 ± 0.06 |

Group II repairs utilizing the cyanoacrylate-scaffold composite all performed extremely well. Bonds formed using the Group II composites were on average 34% stronger than Group I and III organ repairs and 24% stronger than Group I and III vascular repairs.

Group III repairs performed utilizing a single 4-0 suture revealed the high variability in tensile strength associated with this repair technique. This method is highly dependent upon operator skill and technique as indicated by the large standard deviations seen within each tissue group; as well as, tissue type. Considering organ repairs (FIG. 1) only: mean standard deviations for all tissue types in Group I, Group II and Group III, were 7%, 6% and 30%, respectively. Considering vascular repairs (FIG. 2) only: mean standard deviations for all tissue types in Group I, Group II and Group III, were 6%, 6% and 22%, respectively. Gall bladder, liver, lung, and pancreas suture repairs yielded particularly low tensile strengths compared to native tissue, 28%, 31%, 31%, and 35% respectively.

### EXAMPLE 2

### Scaffold Enhanced Use Of 2-Octyl-Cyanoacrylate Versus Sutures In Strabismus Surgery

Traditional strabismus surgery is time-consuming and technically demanding. Specialized spatulated needles must be passed mid-depth through a curved sclera that can be as little as 0.3 mm thick. Inadvertent ocular penetration during surgery can lead to blinding complications such as retinal detachment, vitreous hemorrhage and possibly endophthalmitis. A sutureless bioadhesive would eliminate many potential complications.

2.1 Surgical Procedure

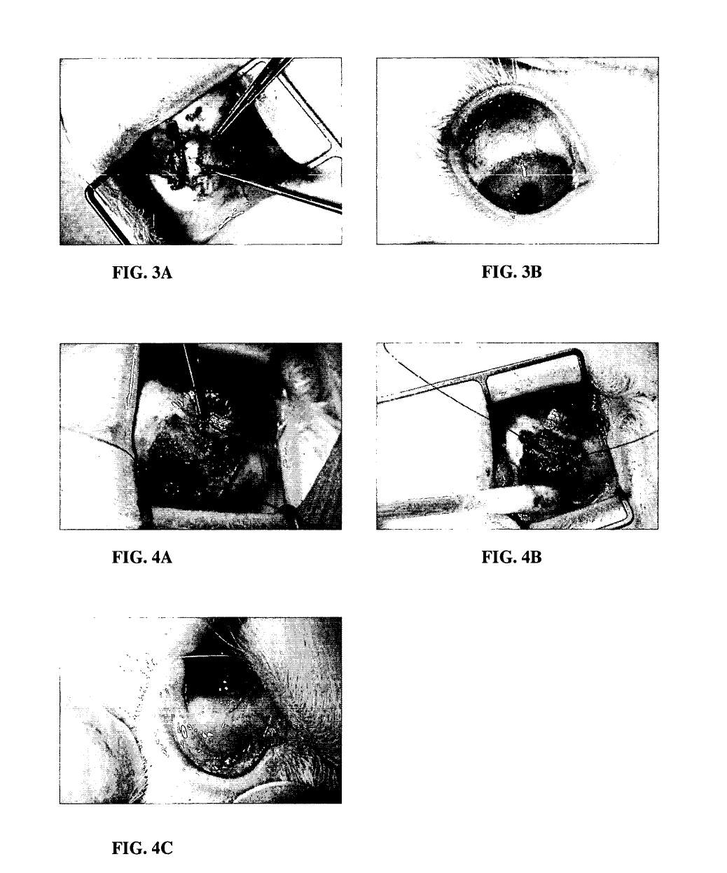

Rabbit (n=12) superior rectus muscles (n=24) were isolated, severed from their scleral insertions and recessed to a point 4.0 mm from the corneoscleral limbus. Three experimental groups based on the method of repair were designated. The ‘Suture’ group utilized standard 6-0 polyglycolic acid sutures with spatulated needles to reattach muscles. The ‘Glue’ group utilized 2-octyl-cyanoacrylate applied directly to the sclera with the spread-out tendon (superior rectus muscle) held in the desired position (FIG. 3A) until the adhesive had set (approx. 20 seconds). The ‘Composite’ group utilized a porous poly(L-lactic-co-glycolic acid) membrane to act as a scaffold for the glue between the muscle and sclera. The superior rectus muscles were isolated and the scaffold was glued in a predetermined position on the sclera using cyanoacrylate glue (FIG. 4A). Cyanoacrylate glue was then placed on the scaffold and the muscle was laid in the desired position (FIG. 4B).

2.2 Evaluation Techniques

Half of the animals were sacrificed at 2 days and the remainder were sacrificed at 14 days after surgery (FIGS. 3B and 4C). At each time point, half of the attachments immediately underwent tensile strength testing on an Instrom material strength testing machine and the other half were processed for histological examination.

2.3 Results

The results of the tensile strength analysis are shown below in Table 3.

TABLE 3

| | Tensile Strength (N) | | | |
|---|---|---|---|---|
| | | | | Cyanoacrylate |
| | Un-operated | Single 6-0 | Cyanoacrylate | Glue + Scaffold |
| Evaluation Period | Controls | Suture | Glue | Composite |
| 2 days | 2.73 ± 1.23 | 298 ± 1.07 | 1.96 ± 1.35 | 1.88 ± 0.50 |
| 14 days | 2.73 ± 1.23 | 2.02 ± 2.13 | 2.17 ± 0.13 | 2.36 ± 0.08 |

As shown in Table 3, preliminary experiments utilizing a glue+scaffold composite to reattach muscles following recession are encouraging. All attachments made using the composite maintained tensile strengths above that needed in humans following recession surgery. [Collins et al., Invest. Ophthal. Vis. Sci., 20:652-64, 1981] Additionally, the technique using the composite had improved ease of application which yielded more uniform results, as is reflected in the reduced variability compared to the other repair techniques evaluated. FIGS. 3B and 4C show the typical postoperative appearance of the eyes 14 days after strabismus surgery using cyanoacrylate glue alone (FIG. 3B) and scaffold-enhanced cyanoacrylate glue (FIG. 4C).

Histologic examination of muscle insertions at 14 days showed no significant signs of inflammation in any of the groups. Muscle-sclera attachments were histologically similar to control insertions. Clinically, all animals tolerated the surgery well with minimal clinical signs of inflammation. The ‘Composite’ group provided a more accurate placement of the muscle compared to ‘Glue’ alone. It also provided more consistent tensile strength than either ‘Suture’ or ‘Glue’ alone.

### EXAMPLE 3

### Composites Containing Cyanoacrylate Adhesives and Biodegradable Scaffolds: In Vivo Wound Closure Study in a Rat Model

3.1 Summary

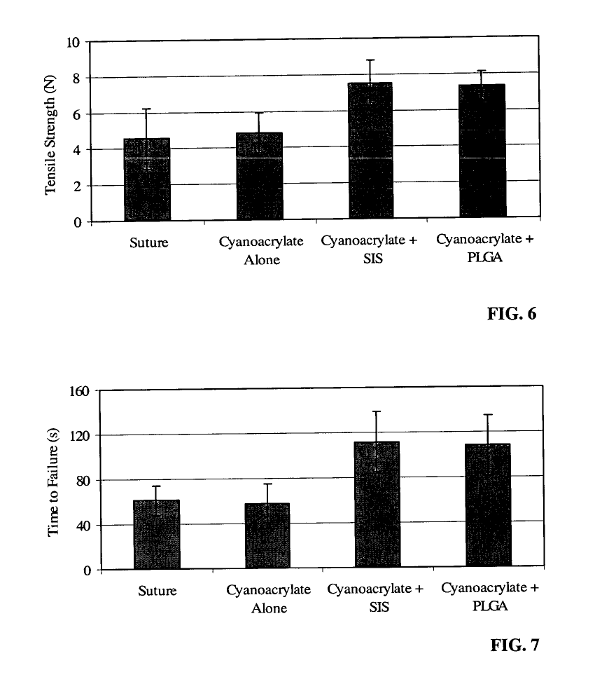

Composites comprising biodegradable scaffolds doped with a cyanoacrylate adhesive were investigated for use in wound closure as an alternative to using cyanoacrylate adhesives alone. Two different scaffold materials were investigated: (i) a biological material, small intestinal submucosa (SIS), manufactured by Cook BioTech; and (ii) a synthetic biodegradable material fabricated from poly(L-lactic-co-glycolic acid) (PLGA). Ethicon's Dermabond™, a 2-octyl-cyanoacrylate, was used as the adhesive. The tensile strengths of skin incisions repaired in vivo in a rat model were measured at seven and fourteen days postoperatively, and the time to failure was recorded. Incisions closed by suture or by cyanoacrylate alone were also tested for comparison. Finally, a histological analysis was conducted to investigate variations in wound healing associated with each technique at seven and fourteen days postoperatively. Data relating to Example 3 is shown in Tables C, D, E, and F of the Appendix, and in FIGS. 6, 7, 8A-8C, 9 and 10, as described below.

3.2 Materials and Methods

3.2.1 Preparation of PLGA Scaffolds

Porous synthetic polymer scaffolds were prepared from PLGA, with a lactic:glycolic acid ratio of 50:50, using a solvent-casting and particulate leaching technique. The scaffolds were cast by dissolving 200 mg PLGA (Sigma Chemical Company, St. Louis, Mo.) in 2 ml dichloromethane (Sigma Chemical Company). Sodium chloride (salt particle size: 106-150 μm) with a 70% weight fraction was added to the polymer mix. The polymer solution was then spread to cover the bottom surface of a 60 mm diameter Petri dish that was cleaned first with dichloromethane, then ethanol, then ultra-filtered deionized water (Fisher Scientific, Pittsburgh, Pa.). The polymer was left in a fume hood for 24 hours to allow the dichloromethane to evaporate. The salt was leached out of the polymer scaffolds by immersion in filtered deionized water for 24 hours, to create the porous scaffolds. During this period the water was changed 3-4 times. The scaffolds were then air dried and stored at room temperature until required. The PLGA scaffolds were cut into rectangular pieces with dimensions of 15±0.5 mm long by 10±0.5 mm wide. The average thickness of the scaffolds, determined by scanning electron microscopy and measurement with precision calipers, was 150±5 μm. Prior to use for tissue repair, the scaffolds were soaked in saline for a period of at least 10 minutes.

3.2.2 Preparation of SIS Scaffolds

SIS is prepared from decellularized porcine submucosa, which essentially contains intact extracellular matrix proteins, of which collagen is the most prevalent. Sheets of SIS, with surface dimensions of 50×10 cm and an average thickness of 100 μm, were provided by Cook BioTech (Lafayette, Ind.). The sheets were cut into rectangular pieces with dimensions of 15±0.5 mm long by 10±0.5 mm wide, and rehydrated in saline for at least 10 minutes prior to being using for tissue repair.

3.2.3 Surgical Repair

Eighteen Wistar rats, weighing 450±50 g, were anesthetized with a mixture of ketamine and xylazine. Four 15 mm long incisions were then made on the dorsal skin of each rat using a #15 scalpel blade: (1) left rostral parasagital; (2) right rostral parasagital; (3) left caudal parasagital; and (4) right caudal parasagital. Each incision site was randomly assigned to a one of the four repair techniques to be investigated.

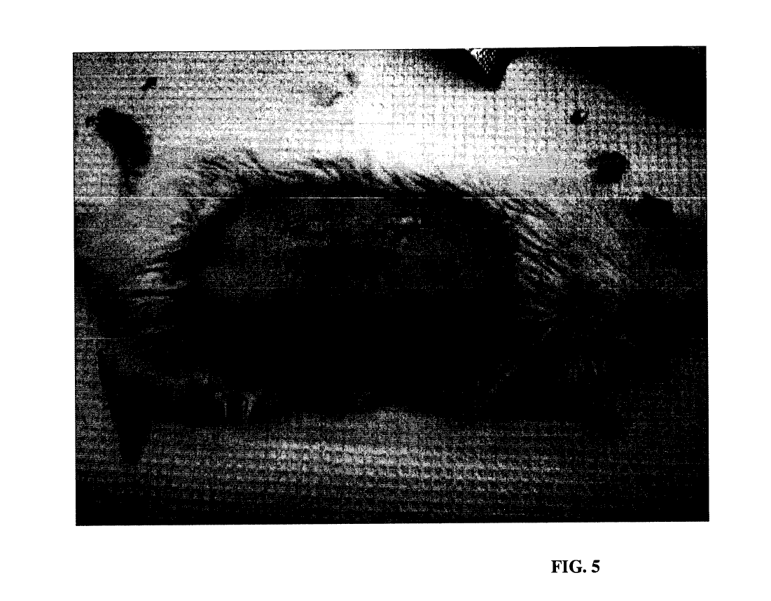

The “Suture” group utilized three, equally spaced interrupted 5-0 polyglycolic acid (Vicryl) sutures. The “Cyanoacrylate alone” group was closed in accordance with the directions provided in the packaging by Ethicon, Inc. One-half an ampoule (˜0.175 mL) was used for each closure. For the “Cyanoacrylate+PLGA” group, five drops of Dermabond (˜0.035 mL) were applied to the irregular surface of the scaffolding using a 26G syringe to create the composite. The composite was then placed across the incision and allowed to air dry (˜10-20s). Finally, for the “Cyanoacrylate+SIS” group, the hydrated SIS specimens were observed to easily fold over on themselves, and were difficult to unravel afterwards. Thus, five drops of Dermabond (˜0.035 mL) were first applied to the incision site, and a piece of hydrated SIS scaffolding was then laid across the Dermabond with its irregular surface against the tissue. FIG. 5 shows a photograph of the incision sites on the dorsal skin of a rat taken immediately following the repair of each incision using one of the four techniques described above. In FIG. 5, the incision on the left rostral parasagital was repaired using a composite including cyanoacrylate and SIS; the incision on the right rostral parasagital was repaired using sutures; the incision on the left caudal parasagital was repaired using a composite including cyanoacrylate and PLGA; and the incision on the right caudal parasagital was repaired using cyanoacrylate alone.

Following the surgical procedure, all animals received a post-operative analgesic dose of buprenorphine. All animals were divided into two groups. Group I (n=13) were observed for seven days after surgery and Group II (n=5) were observed for fourteen days after surgery. At the end of the observation period, all animals were euthanized with pentobarbital and the surgical sites were excised for evaluation. Ten repairs for each wound closure technique from Group I and three repairs for each wound closure technique from Group II were prepared for tensile strength testing. The remaining incision sites that did not undergo strength testing were subjected to histological examination. A summary of incision treatments is given in Table 4:

TABLE 4

| # | Evaluation | Repair | Repair | Repair | Repair |
|---|---|---|---|---|---|
| Animals | Technique | Technique #1 | Technique #2 | Technique #3 | Technique #4 |
| 10 | 7 days - | single 5-0 | cyanoacrylate | cyanoacrylate + | cyanoacrylate + |
| | tensile strength | nylon suture | alone | SIS | PLGA |
| 3 | 7 days - | single 5-0 | cyanoacrylate | cyanoacrylate + | cyanoacrylate + |
| | histology | nylon suture | alone | SIS | PLGA |
| 3 | 14 days - | single 5-0 | cyanoacrylate | cyanoacrylate + | cyanoacrylate + |
| | tensile strength | nylon suture | alone | SIS | PLGA |
| 2 | 14 days - | single 5-0 | cyanoacrylate | cyanoacrylate + | cyanoacrylate + |
| | histology | nylon suture | alone | SIS | PLGA |

3.2.4 Tensile Strength Analysis

The integrity of the resultant repairs were determined by tensile strength measurements performed immediately following the repair procedure using a calibrated MTS Material Strength Testing Machine (858 Table Top System, MTS, Eden Prairie, Minn.). This system was interfaced with a personal computer to collect the data. Each tissue specimen was clamped to the strength-testing machine using a 100N load cell with pneumatic grips. The specimens were pulled apart at a rate of 1 gf/sec until the repair failed. Complete separation of the two pieces of tissue defined failure. The maximum load in Newton's was recorded at the breaking point, as well as the time in seconds to failure. In order to avoid variations in repair strength associated with drying, the tissue specimens were kept moist during the procedure.

3.2.5 Histological Analysis

Light microscopy was used to assess the histological characteristics of wound healing associated with each technique at seven and fourteen days postoperatively. Harvested specimens were immediately fixed in formalin and stored at 6° C. until they could be prepared for staining and mounting. Hematoxylin and Eosin (H&E) was used as the staining agent.

3.3 Results

3.3.1 Wound Healing at Seven Days Postoperatively

The tensile strengths of the repair sites using the four different repair techniques harvested at seven days postoperatively are shown in FIG. 6. The time to failure for each repair procedure at 7 days postoperatively is shown in FIG. 7. All values are expressed as the mean and standard deviation for a total of ten repairs.

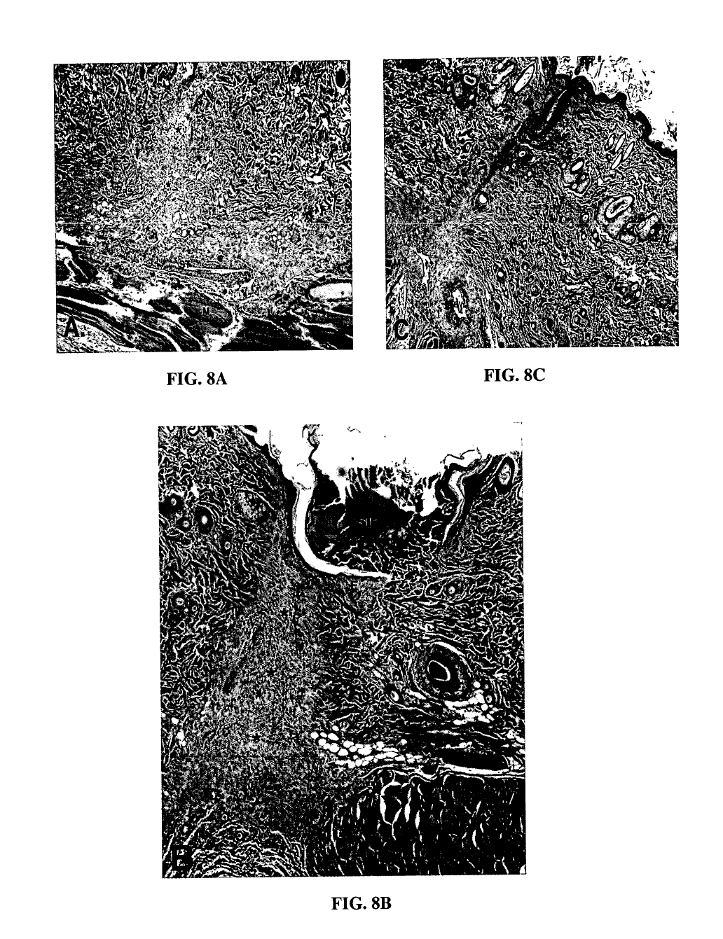

Typical photomicrographs of rat dorsal skin 7 days after standardized full-thickness incision and repair with: (i) 5-0 Nylon suture; (ii) standard external application of cyanoacrylate (Dermabond™); and (iii) external application of PLGA scaffold combined with cyanoacrylate, are shown in FIGS. 8A-8C. Histological examination of repairs made with 5-0 Nylon suture showed minimal inflammation (FIG. 8A). The repair was evidenced by a narrow tract of granulation tissue in the wound bed (*). Inflammation was limited to a low-grade granulomatous type reaction around the suture and suture tract seen at the dermal-subdermal junction. Repairs made with external application of cyanoacrylate alone (FIG. 8B) exhibited a localized superficial inflammatory reaction (SIR). Minimal inflammation was noted in the dermis and wound bed, however, the wound tract and repair was significantly widened. The granulation tissue and width of the repair were increasingly large with progression into the deeper dermis. Finally, repairs made by external application of a PLGA scaffold combined with cyanoacrylate (FIG. 8C) exhibited a minimal superficial inflammatory reaction (keratinized debris, few inflammatory cells). Of note, the wound tract was well apposed with a narrow band of granulation tissue. There was also minimal inflammation in the superficial, middle or deep dermis.

3.3.2 Wound Healing at Fourteen Days Postoperatively

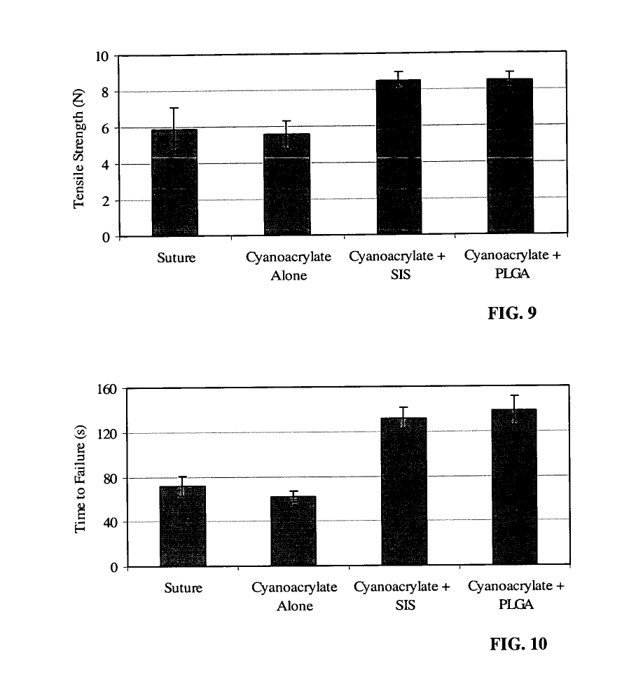

The tensile strengths of the repair sites using the four different repair techniques harvested at fourteen days postoperatively are shown in FIG. 9. The time to failure for each repair procedure at fourteen days postoperatively is shown in FIG. 10. All values are expressed as the mean and standard deviation for a total of three repairs.

3.4 Discussion

Differences in wound healing and tensile strength observed at 7 and 14 days post-operative can likely be explained by the properties of the different techniques.

SUTURES: Wound fixation by interrupted sutures creates a physical apposition of the dermis along the entire length of the wound. However, with any applied forces (including simply the movement and stretch of the skin as the animal moves and performs activities of daily living), the force is concentrated on the individual sutures. This allows differential movement of dermis between sutures and the contact away from the sutures is constantly being stressed, lost and reestablished with the alleviation of stress. In these areas, wound healing will be different and delayed from areas where dermis is kept in constant contact. Therefore, the wound healing between the sutures—which is the majority of the wound area—falls somewhere between true primary intention and secondary intention. Secondary intention healing always results in a longer time to restoration of wound integrity. Although it is sufficient, it is not optimal and at 7 and 14 days there are large areas of the wound that have not healed as well as they would if they were in constant physical apposition and were able to move in concert with externally applied stress.

CYANOACRYLATE: Cyanoacrylate alone performed comparably to that of suture repair. Early on it had less variability than that of sutures. This is likely due to the technical simplicity with which it is effectively applied versus that of the skill required and inherent variability in suture placement. Dermabond acts as a brittle scaffold that bridges the entire wound. This theoretically keeps the wound edges in apposition at all points along the closure. However, as our ex vivo and immediate tensile strength tests have shown, the tensile strength of cyanoacrylate alone is less than for the cyanoacrylate+scaffold composite. Cyanoacrylate is brittle and tends to lose adhesion either through cracking or a separation from the epithelium as an entire sheet when external stress is applied. In this study, early cracking and loss of tight continuous apposition along the entire length of the wound was noted within 24 hours with normal rat daily living activities. Since the animal will twist and bend and stretch the wound, cyanoacrylate is not an optimum method of skin wound closure. When the glue cracks and loses adhesion in focal areas, the healing replicates that of suture healing in that sections of the dermis are separated and must heal by something between true primary and secondary intention. With time, as adhesions are significantly lost, enough native tensile strength has returned to prevent significant numbers of dehiscences, but wound stretching and less cosmetic scar formation occurs along with a decrease in potential wound tensile strength early on.

COMPOSITE: The composite acts to keep the dermis in tight apposition throughout the critical early phase of wound healing when tissue gaps are bridged by scar and granulation tissue. It has the property of being more flexible than cyanoacrylate and may allow the apposed edges to move in conjunction with each other as a unit for a longer period of time and over a greater range of stresses than cyanoacrylate alone. This permits more rapid healing and establishment of integrity since the microgaps between the dermis edges are significantly reduced. By the time the scaffolds are sloughed (by either the animal scratching them off or loss of adhesion to the epithelium) there is greater strength and healing than that produced by cyanoacrylate alone and in wounds following suture removal.

### EXAMPLE 4

### Composites Containing Cyanoacrylate Adhesives and Biodegradable Scaffolds: Acute Wound Closure Study in a Rat Model

4.1 Summary

Composites comprising biodegradable scaffolds doped with cyanoacrylate adhesive were investigated for use in wound closure as an alternative to using cyanoacrylate adhesives alone. Two different scaffold materials were investigated: (i) a biological material, small intestinal submucosa (SIS), manufactured by Cook BioTech; and (ii) a synthetic biodegradable material fabricated from poly(L-lactic-co-glycolic acid) (PLGA). Ethicon's Dermabond™, a 2-octyl-cyanoacrylate, was used as the adhesive. The tensile strengths of skin incisions repaired ex vivo in a rat model were measured, and the time to failure was recorded.

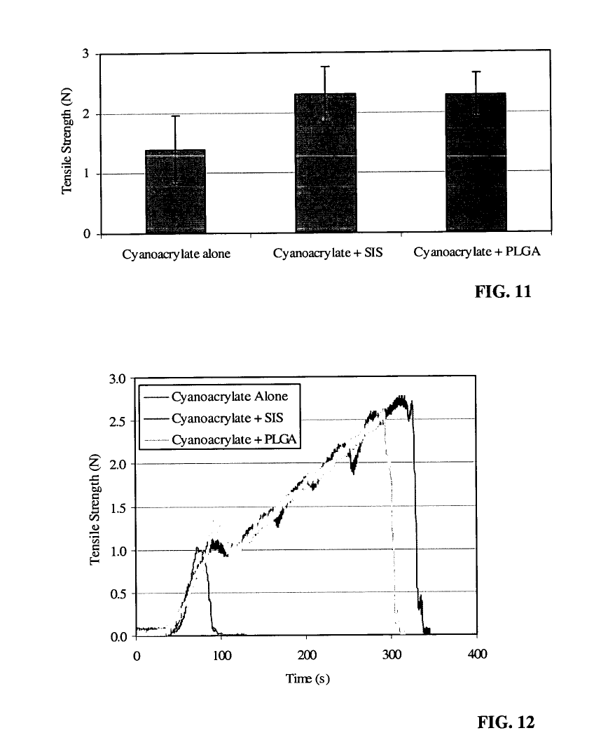

Data relating to Example 4 is shown in Tables G and H of the Appendix, and FIGS. 11-12, as described below.

4.2 Materials and Methods

4.2.1 Preparation of PLGA Scaffolds

Porous synthetic polymer scaffolds were prepared from PLGA, with a lactic:glycolic acid ratio of 50:50, using a solvent-casting and particulate leaching technique. The scaffolds were cast by dissolving 200 mg PLGA (Sigma Chemical Company, St. Louis, Mo.) in 2 ml dichloromethane (Sigma Chemical Company). Sodium chloride (salt particle size: 106-150 μm) with a 70% weight fraction was added to the polymer mix. The polymer solution was then spread to cover the bottom surface of a 60 mm diameter Petri dish that was cleaned first with dichloromethane, then ethanol, then ultra-filtered deionized water (Fisher Scientific, Pittsburgh, Pa.). The polymer was left in a fume hood for 24 hours to allow the dichloromethane to evaporate. The salt was leached out of the polymer scaffolds by immersion in filtered deionized water for 24 hours, to create the porous scaffolds. During this period the water was changed 3-4 times. The scaffolds were then air dried and stored at room temperature until required. The PLGA scaffolds were cut into square pieces with dimensions of 10±0.5 mm long by 10±0.5 mm wide. The average thickness of the scaffolds, determined by scanning electron microscopy and measurement with precision calipers, was 150±5 μm. Prior to use for tissue repair, the scaffolds were soaked in saline for a period of at least 10 minutes.

4.2.2 Preparation of SIS Scaffolds

SIS is prepared from decellularized porcine submucosa, which essentially contains intact extracellular matrix proteins, of which collagen is the most prevalent. Sheets of SIS, with surface dimensions of 50×10 cm and an average thickness of 100 μm, were provided by Cook BioTech (Lafayette, Ind.). The sheets were cut into square pieces with dimensions of 10±0.5 mm long by 10±0.5 mm wide, and rehydrated in saline for at least 10 minutes prior to being using for tissue repair.

4.2.3 Tissue Preparation and Incision Repair

The dorsal skin from thirteen Wistar rats was excised immediately after sacrificing the animals. Rectangular tissue specimens were cut from the skin samples with dimensions of about 20 mm long by 10 mm wide.

A full thickness incision was made with a scalpel across the width of the tissue specimen. Four drops of Dermabond™ were then applied to the irregular surface of the scaffolding using a 27G syringe and the adhesive material was placed across the incision and allowed to air dry. A sample size of ten was used for all experimental groups.

4.2.4 Tensile Strength Analysis

The integrity of the resultant repairs were determined by tensile strength measurements performed immediately following the repair procedure using a calibrated MTS Material Strength Testing Machine (858 Table Top System, MTS, Eden Prairie, Minn.). This system was interfaced with a personal computer to collect the data. Each tissue specimen was clamped to the strength-testing machine using a 100N load cell with pneumatic grips. The specimens were pulled apart at a rate of 1 gf/sec until the repair failed. Complete separation of the two pieces of tissue defined failure. The maximum load in Newton's was recorded at the breaking point, as well as the time profiles for failure of the repairs. In order to avoid variations in repair strength associated with drying, the tissue specimens were kept moist during the procedure. The strengths of corresponding specimens repaired with cyanoacrylate alone, in accordance with the directions provided by Ethicon, Inc., were tested and used as references.

4.3 Results

The tensile strength of the repairs performed in this acute wound closure study using cyanoacrylate alone and a composite including cyanoacrylate enhanced by a scaffold fabricated from either SIS or PLGA, are shown in FIG. 11. All values are expressed as the mean and standard deviation for a total of ten repairs. A comparison of typical time profiles for failure of the repairs is shown in FIG. 12. Each plot represents the mean and standard deviation for ten repairs.

4.4 Discussion

Successful wound closure will occur when dermal edges are kept in physical contact (or with as little gap as possible) so that granulation and scar tissue can result in a continuous integrated matrix from edge to edge. This principle of unobstructed apposition also applies to any non-dermal tissues/surfaces where physical attachment (or reattachment) to another dermal or non-dermal surface is desired. When cyanoacrylate is applied externally to a wound and not allowed to penetrate the reticular dermal level or deeper, it provides a consistent low strength bonding of epidermal surfaces. This keeps the dermal edges in apposition so that wound healing can progress unobstructed. Failure of cyanoacrylate surface closure occurs when either the epithelium (which is loosely attached to the papillary dermis) sloughs off, or the glue loses adhesion to the epithelium for various reasons. These reasons include oil secretion and sloughing of dead surface cells.

The composite formed of either a biocompatible (i.e. PLGA) or biological (i.e. SIS) scaffold and an adhesive provided significantly enhanced tensile strength of the adhesion. This produced a consistently stronger adhesion under standardized constantly increasing tensile strength testing conditions.

The combination of either a biocompatible (i.e. PLGA) or biological (i.e. SIS) scaffold and adhesive also produced different physical characteristics of the adhesion—in a favorable manner. Under constantly increasing tensile stress, force generation curves were prolonged in reaching their peaks. This indicates that adhesions resulting from application of the composite could distribute the forces better and withstand stress for longer periods of time.

The composite including either a biocompatible (i.e. PLGA) or biological (i.e. SIS) scaffold and adhesive also produced different peak-trough behavior of the length-tension curves than the adhesive alone. With the composite, adhesions frequently displayed many mini peaks, without significant troughs, with quick recovery of functional tensile strength. Cyanoacrylate alone almost always produced a single (or infrequently a doublet) peak followed by complete failure of strength and complete physical separation of tissues.

Thus, the composite provides a stronger, more durable and consistent adhesion than the adhesive alone. This theory is also supported by several ex vivo experiments demonstrating enhanced tensile strength of irregular porous versus smooth surface scaffolds in identical tissue repairs (refer to Example 5).

### EXAMPLE 5

### Composites Containing Cyanoacrylate Adhesives and Biodegradable Scaffolds: Surface Selection for Enhanced Tensile Strength in Wound Repair

5.1 Summary

An ex vivo study was conducted to determine the effect of the irregularity of the scaffold surface on the tensile strength of repairs formed using a composite comprising a scaffold and a biological adhesive. Two different scaffold materials were investigated: (i) a synthetic biodegradable material fabricated from poly(L-lactic-co-glycolic acid) (PLGA); and (ii) a biological material, small intestinal submucosa (SIS), manufactured by Cook BioTech. Ethicon's Dermabond™, a 2-octyl-cyanoacrylate, was used as the adhesive. The tensile strength of repairs performed on bovine thoracic aorta, liver, spleen, small intestine and lung, using both the smooth and irregular surfaces of the above materials were measured and the time to failure was recorded.

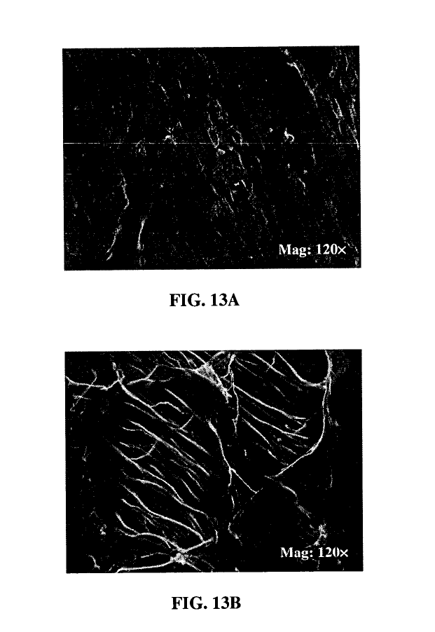

Data relating to Example 5 is shown in Tables I-1, I-2, I-3, I-4, and I-5 of the Appendix, and FIGS. 13A-13B, 14A-14B, 15 and 16, as described below.

5.2 Materials and Methods

5.2.1 Preparation of PLGA Scaffolds

Porous synthetic polymer scaffolds were prepared from PLGA, with a lactic:glycolic acid ratio of 50:50, using a solvent-casting and particulate leaching technique. The scaffolds were cast by dissolving 200 mg PLGA (Sigma Chemical Company, St. Louis, Mo.) in 2 ml dichloromethane (Sigma Chemical Company). Sodium chloride (salt particle size: 106-150 nm) with a 70% weight fraction was added to the polymer mix. The polymer solution was then spread to cover the bottom surface of a 60 mm diameter Petri dish that was cleaned first with dichloromethane, then ethanol, then ultra-filtered deionized water (Fisher Scientific, Pittsburgh, Pa.). The polymer was left in a fume hood for 24 hours to allow the dichloromethane to evaporate. The salt was leached out of the polymer scaffolds by immersion in filtered deionized water for 24 hours, to create the porous scaffolds. During this period the water was changed 3-4 times. The scaffolds were then air dried and stored at room temperature until required. The PLGA scaffolds were cut into square pieces with dimensions of 10±0.5 mm long by 10±0.5 mm wide. The average thickness of the scaffolds, determined by scanning electron microscopy and measurement with precision calipers, was 150±5 mm. Prior to use for tissue repair, the scaffolds were soaked in saline for a period of at least 10 minutes.

5.2.2 Preparation of SIS Scaffolds

SIS is prepared from decellularized porcine submucosa, which essentially contains intact extracellular matrix proteins, of which collagen is the most prevalent. Sheets of SIS, with surface dimensions of 50×10 cm and an average thickness of 100 μm, were provided by Cook BioTech (Lafayette, Ind.). The sheets were cut into square pieces with dimensions of 10±0.5 mm long by 10±0.5 mm wide, and rehydrated in saline for at least 10 minutes prior to being using for tissue repair.

5.2.3 Surface Analysis using Scanning Electron Microscopy

Prior to conducting any tissue repairs, sample surfaces of all scaffolds to be investigated were viewed with a Hitachi S-3000N scanning electron microscope (SEM) to characterize the degree and nature of their smoothness or irregularity.

5.2.4 Tissue Preparation and Incision Repair

Bovine tissue specimens were harvested approximately 30 minutes after sacrificing the animals. Tissue specimens were stored in phosphate buffered saline for a maximum of two hours before they were prepared for experiments. Each tissue specimen was cut into small rectangular pieces with dimensions of about 20 mm long by 10 mm wide and a thickness of approximately 1.5±0.5 mm. Tissue specimens harvested included the thoracic aorta, liver, spleen, small intestine, and lung.

A full thickness incision was made with a scalpel across the width of the tissue specimen. Four drops of Dermabond™ were then applied to the desired surface of the scaffolding (smooth or irregular) using a 26G syringe and the adhesive material was placed across the incision and allowed to air dry. A sample size of ten was used for all experimental groups.

5.2.5 Tensile Strength Analysis

The integrity of the resultant repairs were determined by tensile strength measurements performed immediately following the repair procedure using a calibrated MTS Material Strength Testing Machine (858 Table Top System, MTS, Eden Prairie, Minn.). This system was interfaced with a personal computer to collect the data. Each tissue specimen was clamped to the strength-testing machine using a 100N load cell with pneumatic grips. The specimens were pulled apart at a rate of 1 gf/sec until the repair failed. Complete separation of the two pieces of tissue defined failure. The maximum load in Newton's was recorded at the breaking point, as well as the time in seconds to failure. In order to avoid variations in repair strength associated with drying, the tissue specimens were kept moist during the procedure. The strengths of corresponding native specimens and incisions repaired with cyanoacrylate alone were tested and used as references.

5.3 Results

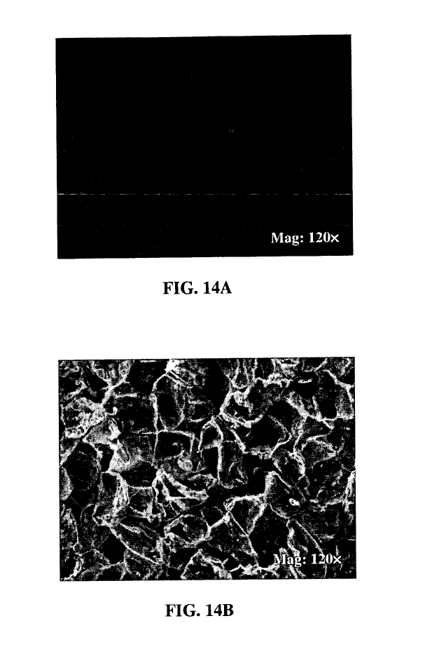

Electron micrographs of both the smooth (intimal) and irregular surfaces of the SIS scaffolds are shown in FIGS. 13A and 13B, respectively. Electron micrographs of both the smooth and irregular surfaces of the PLGA polymer scaffolds are shown in FIGS. 14A and 14B, respectively. The smooth surface of the SIS scaffolds represents the luminal side of the small intestine. The smooth surface of the PLGA scaffolds represents the side of the scaffold that was cast against the surface of the glass Petri dish.

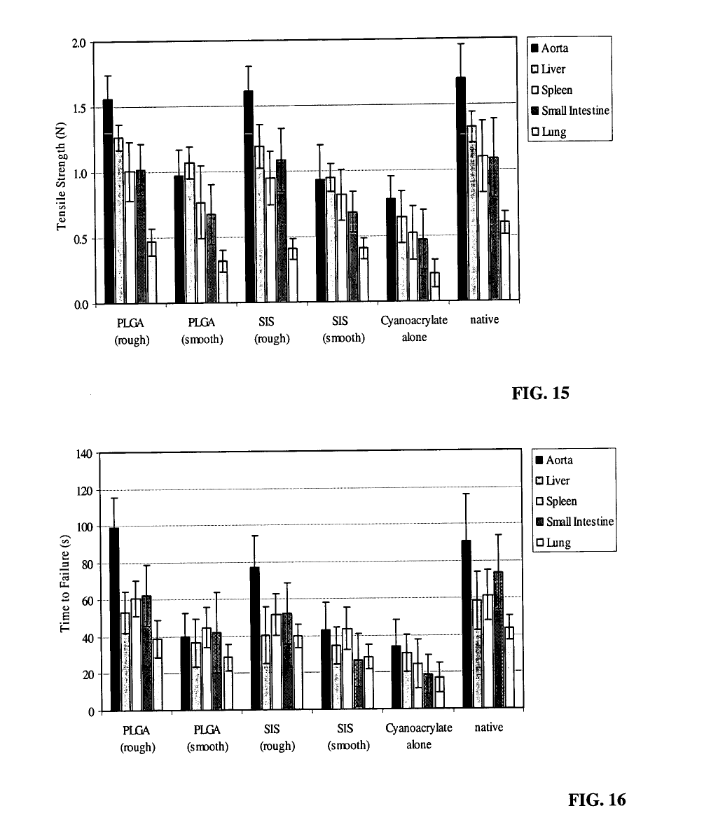

The tensile strength of repairs performed on bovine thoracic aorta, liver, spleen, small intestine and lung, by applying either the smooth or the irregular surfaces of the composites to the tissue surface, are shown in FIG. 15. The time to failure for each repair procedure is shown in FIG. 16. All values are expressed as the mean and standard deviation for a total of ten repairs. The results for incisions repaired with cyanoacrylate alone and for native tissue are also shown.

5.4 Discussion

Several key points are immediately noted from FIGS. 15 and 16.

The irregular, rough surface of the composite provides a greater tensile strength immediately after the adhesion is initiated than does the cyanoacrylate alone, approximating the native tissue strength.

The smooth surface of the composite provides a small increase in tensile strength over cyanoacrylate alone; however, the rough surface of the composite provides a consistently high tensile strength, approximating the native tensile strength of all tissues tested. These results suggest that distributing or dispersing the adhesive forces over an increased surface area of the scaffold, either smooth or rough, can produce better results than cyanoacrylate alone. However, an irregular, rough, or porous surface can significantly increase tensile strength. This presumably occurs by distributing the forces between thousands or millions of independent “microadhesion”.

The clinical relevance of these results is significant. Surgical repairs are more likely to fail in the first hours-to-days after surgery as a result of several factors: a) wound edges are only apposed by whatever artificial means was employed to repair the incision; these methods are subject to the limitations of how they grasp the tissues and anchor them together; b) during the early surgical period, there has not been significant time enough for primary or secondary intention wound healing to provide any native tensile strength to the apposition itself; c) postoperatively edema (which contributes increased forces on the wound, greater than that seen at the time of repair) is greatest in the first 24 hours after surgery (often increasing over this period of time); and d) certain tissues will immediately be subject to high forces after repair/surgery, i.e. aortic pulsatile blood pressure, muscle/tendon contractions against insertions, etc.

All the above factors may contribute to the early postoperatively failure of suture or other methods of repair, such as adhesives or staples. If a tissue repair can achieve a tensile strength approximating the native tensile strength of the tissue in the immediate postoperatively period, the likelihood of failure is markedly diminished and it is certainly much less likely to fail than would a system characterized by more variability and lower tensile strengths.

### EXAMPLE 6

### Composites Containing Cyanoacrylate Adhesives and Biodegradable Scaffolds: Effect of Scaffold Surface Area on Tensile Strength of Repairs

6.1 Summary

An ex vivo study was conducted to determine the effect of varying the area of the scaffold surface in contact with the tissue on the tensile strength of repairs formed using a scaffold-enhanced biological adhesive composite. Biodegradable polymer scaffolds of controlled porosity were fabricated with poly(L-lactic-co-glycolic acid) and salt particles using a solvent-casting and particulate-leaching technique. The scaffolds were doped with Ethicon's Dermabond™, a 2-octyl-cyanoacrylate adhesive. The tensile strength of repairs performed on bovine thoracic aorta and small intestine were measured and the time to failure was recorded.

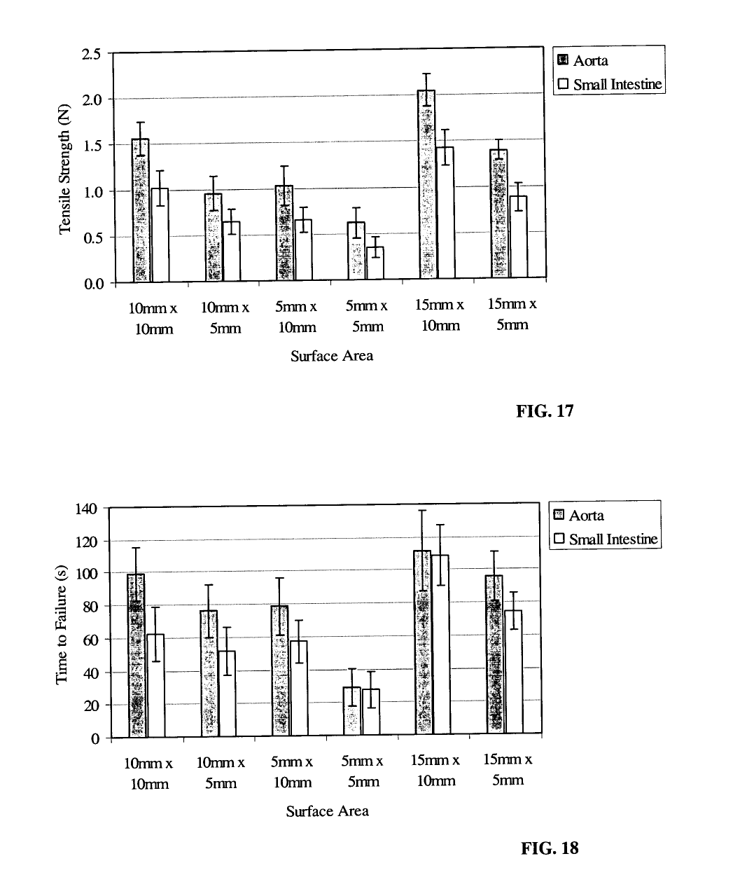

Data relating to Example 6 is shown in Tables J-1 and J-2 of the Appendix, and in FIGS. 17-18, as described below.

6.2 Materials and Methods

6.2.1 Preparation of PLGA Scaffolds

Porous synthetic polymer scaffolds were prepared from PLGA, with a lactic:glycolic acid ratio of 50:50, using a solvent-casting and particulate leaching technique. The scaffolds were cast by dissolving 200 mg PLGA (Sigma Chemical Company, St. Louis, Mo.) in 2 ml dichloromethane (Sigma Chemical Company). Sodium chloride (salt particle size: 106-150 μm) with a 70% weight fraction was added to the polymer mix. The polymer solution was then spread to cover the bottom surface of a 60 mm diameter Petri dish that was cleaned first with dichloromethane, then ethanol, then ultra-filtered deionized water (Fisher Scientific, Pittsburgh, Pa.). The polymer was left in a fume hood for 24 hours to allow the dichloromethane to evaporate. The salt was leached out of the polymer scaffolds by immersion in filtered deionized water for 24 hours, to create the porous scaffolds. During this period the water was changed 3-4 times. The scaffolds were then air dried and stored at room temperature until required. The PLGA scaffolds were cut into rectangular pieces with the desired surface dimensions (length by width): (i) 10±0.5 mm by 10±0.5 mm; (ii) 10±0.5 mm by 5±0.5 mm; (iii) 5±0.5 mm by 10±0.5 mm; (iv) 15±0.5 mm by 10±0.5 mm; and (v) 15±0.5 mm by 5±0.5 mm. The average thickness of the scaffolds, determined by scanning electron microscopy and measurement with precision calipers, was 150±5 μm. Prior to use for tissue repair, the scaffolds were soaked in saline for a period of at least 10 minutes.

6.2.2 Tissue Preparation and Incision Repair

Bovine tissue specimens were harvested approximately 30 minutes after sacrificing the animal. Tissue specimens were stored in phosphate buffered saline for a maximum of two hours before they were prepared for experiments. Each tissue specimen was cut into small rectangular pieces with dimensions of about 20 mm long by 10 mm wide and a thickness of approximately 1.5±0.5 mm. Tissue specimens harvested included the thoracic aorta and small intestine.

A full thickness incision was made with a scalpel across the width of the tissue specimen. Four drops of Dermabond™ were then applied to the irregular surface of the scaffold using a 26G syringe, and the composite was placed across the incision and allowed to air dry. A sample size of ten was used for all experimental groups.

6.2.3 Tensile Strength Analysis

The integrity of the resultant repairs was determined by tensile strength measurements performed immediately following the repair procedure using a calibrated MTS Material Strength Testing Machine (858 Table Top System, MTS, Eden Prairie, Minn.). This system was interfaced with a personal computer to collect the data. Each tissue specimen was clamped to the strength-testing machine using a 100N load cell with pneumatic grips. The specimens were pulled apart at a rate of 1 gf/sec until the repair failed. Complete separation of the two pieces of tissue defined failure. The maximum load in newtons was recorded at the breaking point, as well as the time in seconds to failure. In order to avoid variations in repair strength associated with drying, the tissue specimens were kept moist during the procedure.

6.3 Results

The tensile strength of repairs performed on bovine thoracic aorta and small intestine by applying the irregular surface of the cyanoacrylate-PLGA scaffold composites to the tissue surface, are shown in FIG. 17, as a function of surface area. The time to failure for each repair procedure is shown in FIG. 18. All values are expressed as the mean±standard deviation for a total of ten repairs.

6.4 Discussion

As shown in FIG. 17, there is an increase in the tensile strength of the repairs with increasing surface area. However, geometric dimensions appear to be less important than total surface area. These results are not unexpected. Since there are probably millions of microadhesions that provide the increased tensile strength and the prolonged time to failure, it is simply a matter of supplying enough microadhesions on both sides of the wound. In contrast, in other types of closures (i.e., suture repairs) geometry and precision placement are crucial to maintenance of strength in the repair since (during early wound healing) all forces are concentrated on a very limited number of small focal points in the repair. The composite structure allows for distribution of forces across the entire repair site including beyond the tissue edges, which further reinforces the wound closure. Thus, the same amount of force applied to a sutured wound and to a composite-closed wound will have much less effect on any given area of the composite-repaired wound. This is most likely why the cosmesis of the composite-closed skin incisions was better than for suture or glue alone.

With the composite, a butterfly-bandage effect occurs, i.e., reinforcement of the wound by the combination of the scaffold and glue brought the edges of the incision, along its entire length, into better apposition for an extended period of time, which contributed to a more satisfactory cosmetic healing.

Geometry may not be completely unimportant (as one would expect when dealing with vector forces). However, it may be clinically insignificant. As seen in small intestine repair, less surface area (oriented differently) had a statistically significant effect (p<0.05): 10×10 mm versus 15×5 mm. This is, however, the only result like this and, depending on the size and orientation of the actual tissue in the experiment, it may be a clinically insignificant isolated result. While the rest of the time points reveal that surface area is likely proportional to the increased time to failure, as would be expected, further studies are needed to confirm these results.

### EXAMPLE 7

### Composites Containing Cyanoacrylate Adhesives and Biodegradable Scaffolds: Custom Manufactured Scaffold Surfaces for Improved Tissue Repair

7.1. Summary

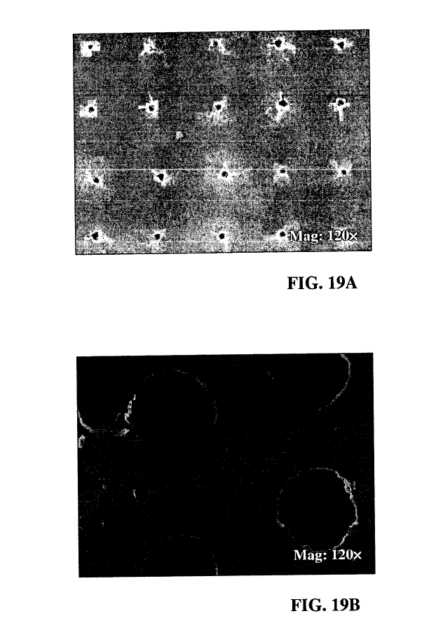

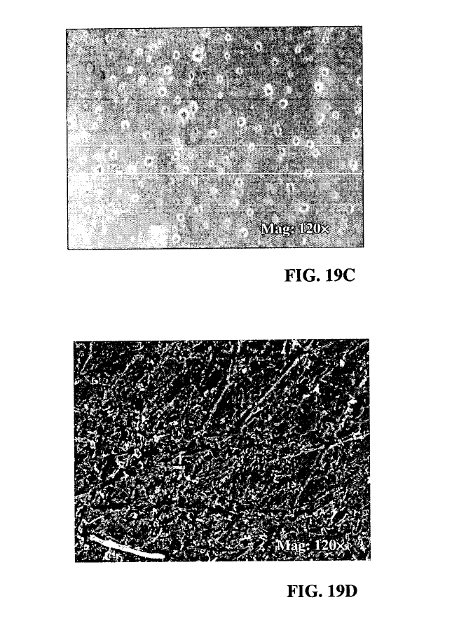

An ex vivo study was conducted to determine the effect of using several different custom modified scaffold surfaces on the tensile strength of repairs formed using our scaffold-adhesive composite. Porous PLGA scaffolds were fabricated using four different manufacturing techniques: (i) a computer-controlled drilling technique; (ii) a punching technique utilizing an arbor press; (iii) a polymer molding technique, and (iv) 220 grit sandpaper. FIGS. 19A-19D show electron micrographs of the irregularities added to the scaffold surface using each of these techniques, respectively. Ethicon's Dermabond™, a 2-octyl-cyanoacrylate, was used as the bioadhesive. The tensile strength of repairs performed on bovine thoracic aorta, liver, spleen, small intestine and lung were measured and the time to failure was recorded. The results of this study were compared with those obtained in a previous study (Example 3 above) using PLGA scaffolds manufactured with a particulate-leaching technique.

Data relating to this Example 7 is shown in Tables K-1, K-2, K-3, K-4 and K-5 of the Appendix, and in FIGS. 19A-19D, 20 and 21, as described below.

7.2 Materials and Methods

7.2.1 Preparation of PLGA Using Various Mechanical Manufacturing Techniques

Synthetic polymer scaffolds were prepared from PLGA, with a lactic:glycolic acid ratio of 50:50. The scaffolds were cast by dissolving 250 mg PLGA in 2.5 ml dichloromethane. The polymer solution was then spread to cover the bottom surface of a 60 mm diameter Petri dish that was cleaned first with dichloromethane, then ethanol, then ultra-filtered deionized water. The polymer was left in a fume hood for 24 hours to allow the dichloromethane to evaporate, and then allowed to soak in filtered deionized water for a period of 2 hours prior to removing from the Petri dish.

Upon drying of the polymer scaffolds, an irregularity was added to the scaffold surfaces using one of four mechanical techniques:
a) Use of a computer numeric control (CNC) machine to punch holes in the scaffold in accordance with a preprogrammed staggered layout. The diameter of each needle was 0.020 in. (500 μm) (FIG. 19A);
b) A punch was created utilizing hundreds of 0.020 in (500 μm) diameter needles, and the punch was then inserted into an arbor press apparatus. Hard rubber was used as a base for the punch (FIG. 19B);
c) A silicone mold was made to provide a textured surface during the casting stage of scaffold manufacture (FIG. 19C); and
d) Use of 220 grit sandpaper to give the scaffold surface a rough texture (FIG. 19D).

The PLGA scaffolds were cut into square pieces with dimensions of 10±0.5 mm long by 10±0.5 mm wide. The average thickness of the scaffolds, determined by scanning electron microscopy and measurement with precision calipers, was 150±10 μm. Prior to use for tissue repair, the scaffolds were soaked in saline for a period of at least 10 minutes.

7.2.2 Surface Analysis using Scanning Electron Microscopy

Prior to conducting any tissue repairs, the surfaces of samples of all scaffolds to be investigated were viewed with a Hitachi S-3000N scanning electron microscope (SEM) to allow characterization of their irregularity.

7.2.3 Tissue Preparation and Incision Repair

Bovine tissue specimens were harvested approximately 30 minutes after sacrificing the animal. Tissue specimens were stored in phosphate buffered saline for a maximum of two hours before they were prepared for experiments. Each tissue specimen was cut into small rectangular pieces with dimensions of about 20 mm long by 10 mm wide and a thickness of approximately 1.5±0.5 mm. Tissue specimens harvested included the thoracic aorta, liver, spleen, small intestine, and lung.

A full thickness incision was made with a scalpel across the width of the tissue specimen. Four drops of Dermabond™ were then applied to the rough surface of the scaffolding using a 26G syringe, and the adhesive material was placed across the incision and allowed to air dry. A sample size of five was used for all experimental groups.

7.2.4 Tensile Strength Analysis

The integrity of the resultant repairs was determined by tensile strength measurements performed immediately following the repair procedure using a calibrated MTS Material Strength Testing Machine (858 Table Top System, MTS, Eden Prairie, Minn.). This system was interfaced with a personal computer to collect the data. Each tissue specimen was clamped to the strength-testing machine using a 100N load cell with pneumatic grips. The specimens were pulled apart at a rate of 1 gf/sec until the repair failed. Complete separation of the two pieces of tissue defined failure. The maximum load in Newton's was recorded at the breaking point, as well as the time in seconds to failure. In order to avoid variations in repair strength associated with drying, the tissue specimens were kept moist during the procedure.

7.3 Results

Electron micrographs of the PLGA polymer scaffolds given an irregular surface using one of the four mechanical techniques described above are shown in FIGS. 19A-19D. All photomicrographs were taken of the rough (most irregular) surface of the scaffolds.

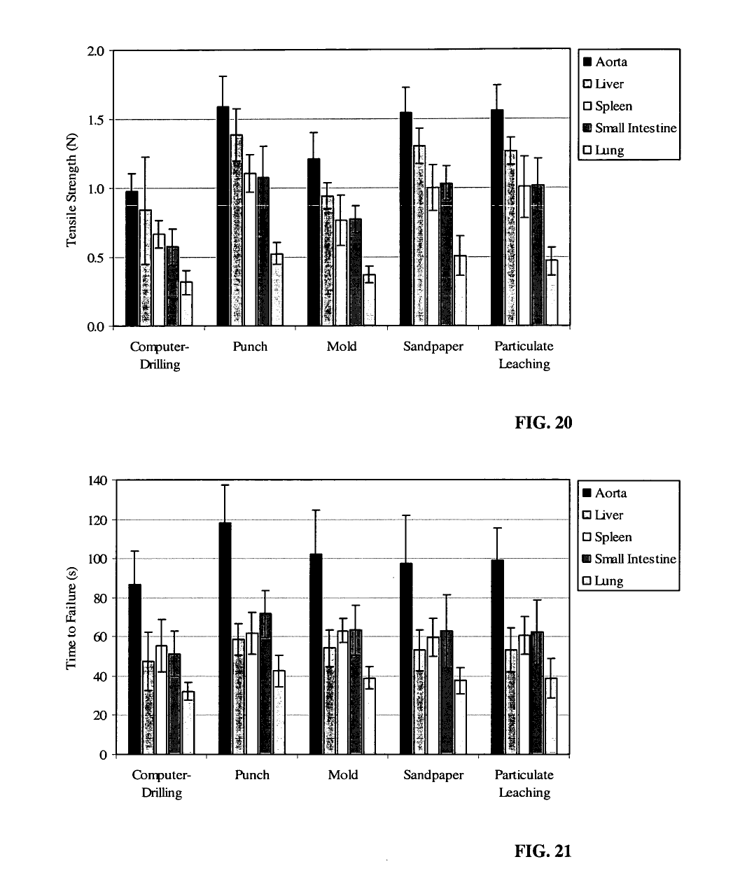

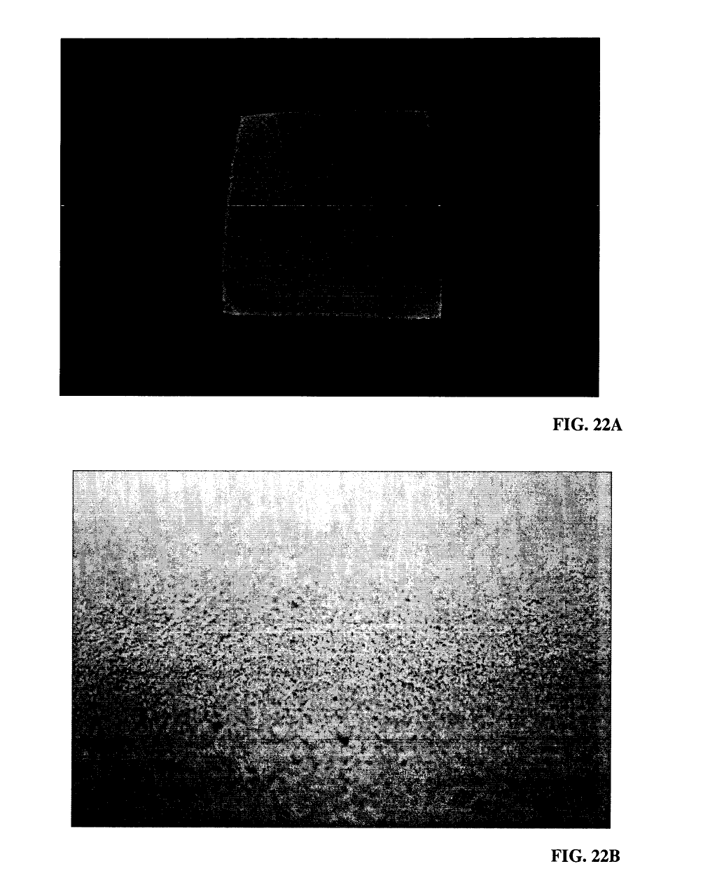

The tensile strength of repairs performed on bovine thoracic aorta, liver, spleen, small intestine and lung, using the cyanoacrylate-scaffold composites described above, are shown in FIG. 20. The time to failure for each repair procedure is shown in FIG. 21. The tensile strength and the time of failure for repairs formed using the irregular surface of the PLGA scaffolds manufactured with the particulate leaching technique of Example 3 are also included for comparison.

7.4 Discussion

As can be seen in the photomicrographs, irregular scaffold surfaces can be manufactured to different specifications of irregularity and porosity, in order to suit various surgical requirements. The photomicrographs of the PLGA scaffolds produced using the punch and sandpaper techniques show the greatest areas of troughs, where the tissue would be in direct contact with the adhesive rather than the scaffold material. Repairs formed using scaffolds manufactured using the punch and sandpaper techniques were the strongest of the four custom manufactured scaffolds investigated (FIG. 20). The strength of these repairs were statistically equivalent (p<0.05) to the strength of repairs formed using scaffolds manufactured with the particulate-leaching technique described in Example 3, with a tendency seen for an increase in tensile strength with the use of the punch technique. The photomicrograph of the computer-drilled PLGA appears to have a smoother surface than the silicone mold PLGA product, while the individual pore sizes are approximately the same. As can be seen in FIG. 20, there is less tensile strength for the computer-drilled scaffold than the scaffold formed with the silicone mold, which is much more irregular and possibly more porous. The above findings support our hypothesis that irregularity and possibly (irregular) porosity contribute to the previously unrecognized synergistic increase in tensile strength of the irregular scaffold over both smooth scaffolds and adhesive alone.

Clinical relevance is less apparent here, other than as support to our theory described in Example 3. However, this finding suggests that many aspects of these scaffolds may be custom manufactured, including porosity (including pore size and distribution), roughness, non-geometric topography (irregularity), to ensure reproducibility of results and to meet the needs of specific applications.

Future studies may be directed at determining whether different surfaces actually work better with one type of adhesive versus another or with adhesives of different viscosity allowing deeper penetration into the depth of the surface irregularities.

As a result of these and other studies, it has been found that a non-light activated adhesive-scaffold composite, incorporating a biological, biocompatible, or biodegradable adhesive and a biological, biocompatible, or biodegradable scaffold, exhibits significantly enhanced tensile strength and consistently stronger adhesion under constantly increasing time periods of tensile strength testing. Also, the composite exhibits more favorable adhesion characteristics. When subjected to constantly increasing loads, the composites exhibited force generation curves that were prolonged in reaching their peaks, indicating better distribution of forces. This allowed the composites to withstand stress for longer periods of time.

Additionally, length-tension curves for the composites are remarkably different than those for bioadhesives alone (e.g., cyanoacrylate). While the bioadhesive alone frequently produced a single peak followed by a trough (indicating complete failure of strength and complete physical separation of tissues), the composite curve showed many peaks without significant troughs (indicating quick recovery of functional tensile strength and little-to-no tissue separation) (FIG. 12).

The specifications of the composite of the present invention can be tailored to meet the specific requirements of a range of clinical applications, such as wound closure from trauma or at surgical incision sites, repair of liver, spleen, or pancreas lacerations from trauma, dural laceration/incision closure, pneumothorax repair during thoracotomy, sealing points of vascular access following endovascular procedures, vascular anastomoses, tympanoplasty, endoscopic treatment of gastrointestinal ulcers/bleeds, dental applications for mucosal ulcerations or splinting of injured teeth, ophthalmologic surgeries, tendon and ligament repair in orthopedics, and episiotomy/vaginal tear repair in gynecology. Patches prepared using the adhesive composites can be used in a non-surgical setting as a simple, quick, and effective wound closure solution, for example, in emergency situations.

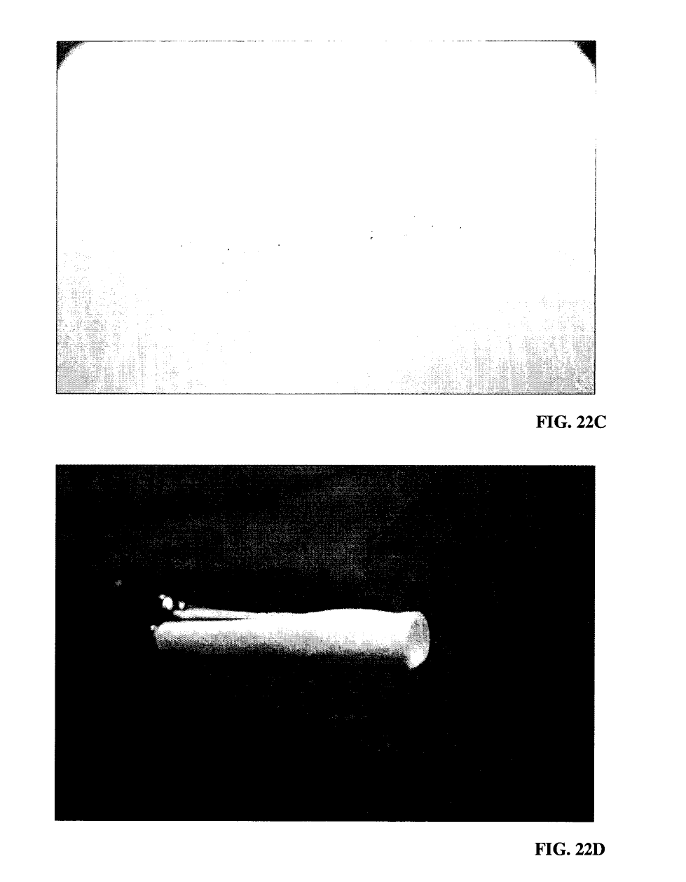

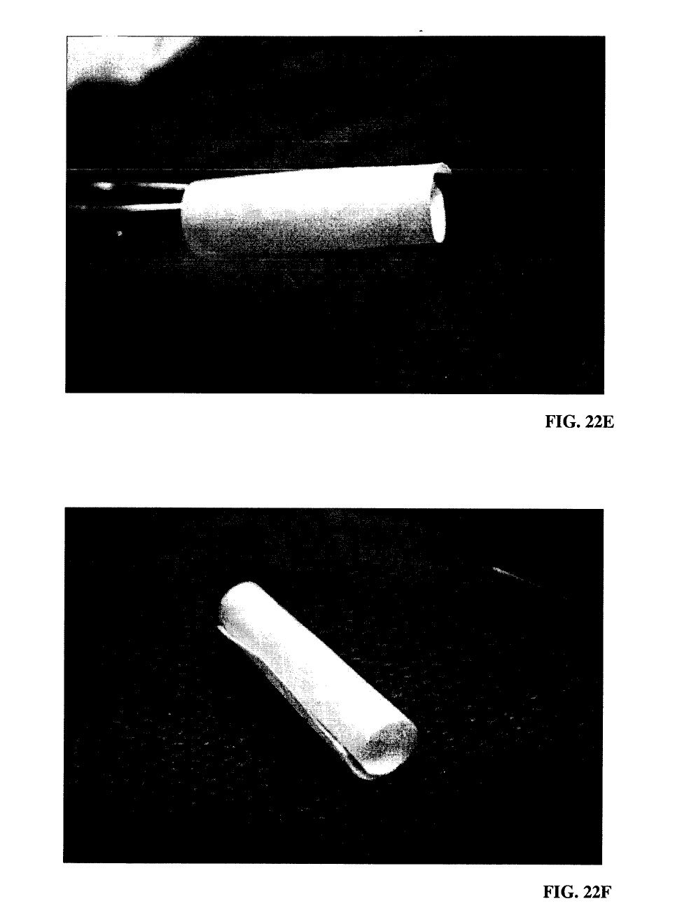

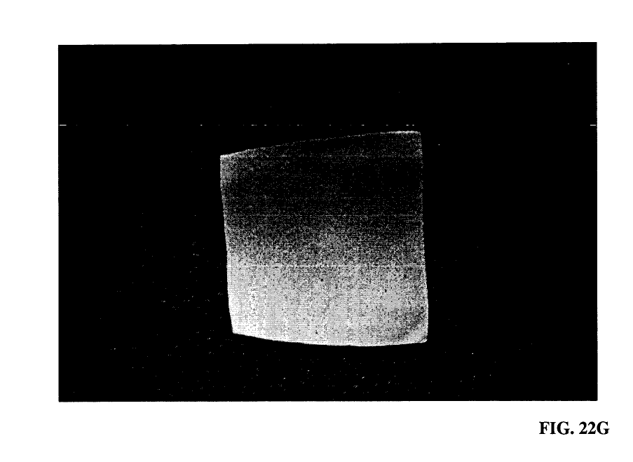

FIGS. 22A-22G show photographs of exemplary embodiments of a scaffold suitable for use in the composite discussed above. In the illustrated embodiment, the scaffold has a rectangular or square shape. FIG. 22A shows that the scaffold may take the form of a thin wafer or sheet. FIG. 22B shows that at least a portion of the scaffold's surface may be irregular, and FIG. 22C shows that at least a portion of the scaffold surface may be smooth. As discussed above, it is understood that different embodiments of the composite may take a variety of forms and/or shapes.

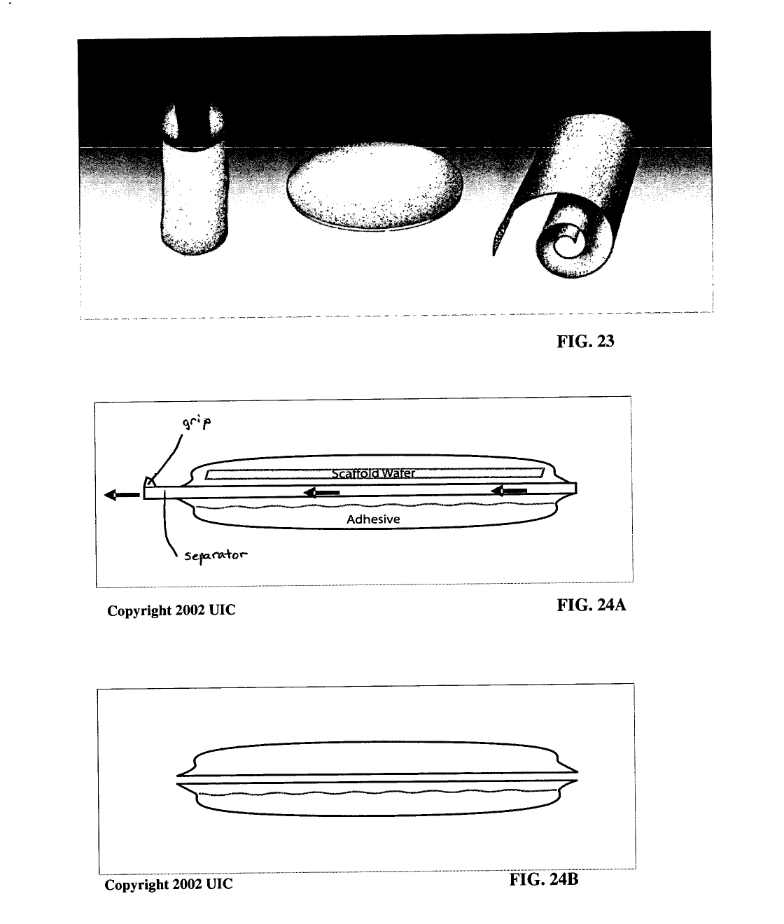

FIGS. 22D and 22E show that the scaffold may be rolled in a tight roll (FIG. 22D) or a loose or wide roll (FIG. 22E) to adapt to various applications, without any damage to its structural integrity. FIG. 22F shows how the scaffold may retain its rolled shape after an elapse of time. FIG. 22G shows that the scaffold may be unrolled after being rolled, and still retain its structural integrity. Additionally, the scaffold may be bent or folded as may be suitable for a particular application. FIG. 23 shows a schematic representation of the some of the above-listed embodiments.

The composite of the present invention may be created by a variety of methods or techniques. For example, a physician or other health care provider may place the scaffold in the desired position for tissue repair, sealing, or adhesion, then apply the adhesive to the scaffold. Alternatively, the adhesive may be applied to the scaffold and then the device containing both scaffold and adhesive placed in position. As another alternative, the adhesive may be placed at the repair site first and then the scaffold applied. Additional adhesive material may be applied to the site before or after the scaffold is positioned. It is understood that the terms “placed” and “positioned” include applying an adhesive and/or scaffold on a wound, tissue, or repair site, across edges of a wound or incision, and/or across a juncture between tissue and a biocompatible implant to be joined or adhered.

The composite of the present invention may be designed and packaged in a variety of different ways. For example, in one embodiment, the composite is packaged in an inert cellophane-like material. The inert material peels off the surface of the composite to allow immediate use. The packaged item may be made available in a variety of sizes and shapes as appropriate for various uses or applications.

In another embodiment, the composite is supported by one or two rollers made of an inert material. The rollers may be configured to be disposable or reusable. The composite is wrapped around the roller or rollers to form a scroll. The scroll is unrolled to apply the composite to a wound or repair site; for example, a curved or irregular surface. A double roller scroll is particularly advantageous in a non-sterile setting (such as an emergency setting, where surgical/sterile gloves are not available), since it avoids the need for a person to directly handle the composite. A single roller scroll is particularly suitable for sterile environments, for example, during surgery, where a gloved hand may be used to position the edge of the composite prior to unrolling.

Yet another alternative packaging technique involves positioning a thin, expendable, fracturable membrane on top of the composite in such a way that the thin membrane protects the composite until it is ready to be used. Upon application of the composite to a wound or repair site, the expendable membrane ruptures or fractures, for example, to expose the adhesive to the desired tissue site.

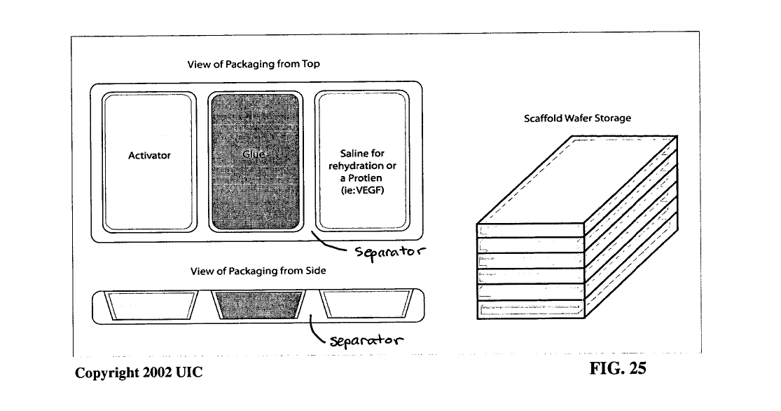

Further alternative embodiments involve the use of a separator, such as an inert tab made of plastic, paper, or other suitable material, to which a grip, for example a ring (similar to that used in laser printer cartridges), is attached. In one such alternative embodiment, a separator is positioned between the scaffold and the adhesive to isolate the scaffold from the adhesive until the composite is needed for application to a wound or repair site (FIG. 24A). Exertion of force on the grip, e.g., in the direction of the arrows shown in FIG. 24A, removes the separator (FIG. 24B), enabling immediate use of the composite.

In another such alternative embodiment, the separator is positioned between the adhesive and an adhesive activator to isolate the adhesive from its activator until the composite is needed for use (FIG. 25). In the embodiment of FIG. 25, a saline or protein, e.g., VEGF, is also included in the composite as shown. The right-hand side of FIG. 25 shows how the packaged composite may be stacked for storage.

In yet another such alternative embodiment, two separators may be provided. A first separator may be positioned between the scaffold and the adhesive, and a second separator positioned between the adhesive and the activator. In this embodiment, one grip may be provided to remove the separator between the activator and adhesive in order to activate the adhesive, and then a second grip may be provided to remove the separator between the adhesive and scaffold, to enable contact between the adhesive and the scaffold. This design may be useful in situations where it may be necessary or desirable to activate the adhesive a certain amount of time prior to application of the composite to the wound or repair site. Alternatively, one grip may be provided, which operates to remove both separators at once.

The composite can be modified to provide biologically active materials to biological tissue. The controlled release of various dopants including hemostatic and thrombogenic agents, antibiotics, anesthetics, various growth factors, enzymes, anti-inflammatories, bacteriostatic or bacteriocidal factors, chemotherapeutic agents, anti-angiogenic agents and vitamins can be added to the composite to assist in the therapeutic goal of the procedure. The degradation rate of the composite, and consequently the drug delivery rate, can be controlled by altering the macromolecular structure of the device or a portion thereof.

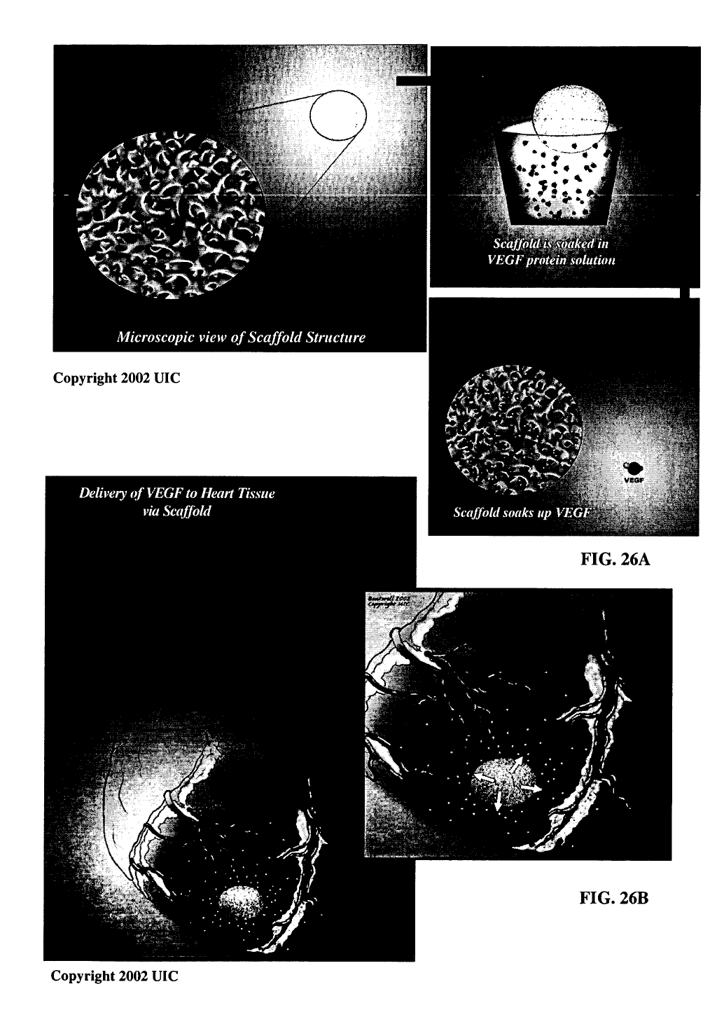

FIGS. 26A and 26B show an example of how the composite may be used to deliver VEGF to heart tissue after surgery. It is understood that similar techniques may be used in the repair of other internal or external wounds. FIG. 26A shows one embodiment in which the scaffold is immersed in VEGF protein. As a result, the scaffold absorbs the VEGF. When combined with the adhesive to form the composite, the composite is then able to release the VEGF to biological tissue when used to repair a wound, for example, as shown in FIG. 26B. It is understood that variations exist in the way the biologically active material is combined with the composite and that such variations are within the scope and spirit of the present invention.

Furthermore, the elasticity, strength, and flexibility of the composite can be modified to meet the demands of and enhance clinical applicability in a wide range of applications. For example, alteration of composition and pore size modifies pliability and elasticity, making it easier to process and fabricate the composite, for example, into different forms and shapes for different applications.

Although specific illustrated embodiments of the invention have been disclosed, it is understood by those skilled in the art that changes in form and details may be made without departing from the spirit and scope of the invention. The present invention is not limited to the specific details disclosed herein, but is to be defined by the appended claims.

TABLE A
Data relating to Example 1, summarized in Table 1 and FIG. 1

| | Tensile Strength (N) | | | |
|---|---|---|---|---|
| | Solder + | Cyanoacrylate + | Single 4-0 | Native |
| Tissue | Scaffold | Scaffold | Suture | Tissue |
| small intestine | 0.91 | 0.96 | 0.16 | 1.06 |
| | 0.79 | 1.04 | 0.47 | 1.13 |
| | 0.84 | 0.88 | 0.04 | 1.02 |
| | 1.00 | 0.87 | 0.57 | 1.20 |
| | 0.90 | 0.80 | 0.76 | 1.11 |
| | 0.75 | 1.02 | 0.12 | 0.90 |
| | 0.78 | 0.89 | 0.96 | 1.16 |
| | 0.90 | 0.94 | 0.25 | 1.05 |
| | 0.94 | 0.85 | 1.13 | 1.01 |
| | 0.83 | 1.00 | 0.41 | 1.08 |
| spleen | 0.72 | 0.93 | 0.37 | 0.86 |
| | 0.57 | 0.83 | 0.27 | 0.83 |
| | 0.62 | 0.78 | 0.67 | 0.92 |
| | 0.65 | 1.00 | 0.80 | 0.88 |
| | 0.69 | 0.90 | 0.84 | 0.94 |
| | 0.61 | 0.86 | 0.21 | 0.97 |
| | 0.63 | 0.98 | 0.75 | 0.95 |
| | 0.70 | 0.94 | 0.48 | 0.81 |
| | 0.68 | 0.84 | 0.18 | 0.90 |
| | 0.64 | 0.96 | 0.60 | 0.94 |
| skeletal muscle | 1.28 | 1.47 | 1.60 | 1.91 |
| | 1.10 | 1.62 | 1.55 | 1.78 |
| | 0.94 | 1.72 | 1.08 | 1.85 |
| | 1.16 | 1.39 | 1.75 | 1.99 |
| | 1.53 | 1.56 | 0.50 | 1.95 |
| | 1.06 | 1.43 | 1.87 | 1.88 |
| | 1.26 | 1.39 | 0.61 | 1.80 |
| | 0.95 | 1.56 | 0.46 | 1.93 |
| | 1.38 | 1.59 | 1.24 | 2.01 |
| | 1.29 | 1.66 | 1.46 | 1.93 |
| skin | 1.06 | 1.43 | 1.68 | 1.67 |
| | 1.22 | 1.46 | 1.83 | 1.59 |
| | 0.95 | 1.50 | 1.19 | 1.50 |
| | 0.97 | 1.60 | 1.06 | 1.63 |
| | 1.03 | 1.58 | 0.99 | 1.70 |
| | 0.91 | 1.48 | 1.99 | 1.72 |
| | 1.01 | 1.40 | 2.12 | 1.64 |
| | 1.10 | 1.59 | 1.40 | 1.69 |
| | 1.03 | 1.50 | 2.18 | 1.67 |
| | 0.89 | 1.44 | 1.36 | 1.60 |
| atrium | 1.02 | 1.25 | 0.34 | 1.29 |
| | 0.78 | 1.27 | 0.94 | 1.36 |
| | 0.98 | 1.16 | 0.18 | 1.42 |
| | 0.94 | 1.14 | 0.78 | 1.33 |
| | 0.75 | 1.18 | 1.01 | 1.29 |
| | 0.97 | 1.20 | 0.44 | 1.44 |
| | 0.91 | 1.23 | 0.90 | 1.36 |
| | 0.85 | 1.26 | 0.34 | 1.32 |
| | 0.80 | 1.13 | 0.82 | 1.34 |
| | 0.91 | 1.19 | 0.26 | 1.29 |
| ventricle | 0.90 | 1.01 | 0.41 | 1.42 |
| | 0.82 | 1.11 | 0.59 | 1.38 |
| | 0.94 | 1.17 | 1.12 | 1.33 |
| | 0.80 | 1.11 | 0.97 | 1.48 |
| | 0.73 | 1.15 | 1.53 | 1.39 |
| | 0.83 | 1.06 | 1.70 | 1.58 |
| | 0.70 | 1.16 | 0.30 | 1.39 |
| | 0.78 | 1.19 | 1.24 | 1.46 |
| | 0.86 | 1.12 | 0.80 | 1.34 |
| | 0.89 | 0.96 | 0.74 | 1.45 |
| lung | 0.18 | 0.46 | 0.07 | 0.75 |
| | 0.21 | 0.53 | 0.37 | 0.74 |
| | 0.25 | 0.55 | 0.11 | 0.71 |
| | 0.34 | 0.52 | 0.08 | 0.73 |
| | 0.16 | 0.42 | 0.48 | 0.72 |
| | 0.19 | 0.51 | 0.55 | 0.75 |
| | 0.22 | 0.60 | 0.21 | 0.68 |
| | 0.17 | 0.45 | 0.54 | 0.70 |
| | 0.24 | 0.57 | 0.04 | 0.66 |
| | 0.27 | 0.41 | 0.10 | 0.69 |
| pancreas | 0.27 | 0.99 | 1.43 | 1.27 |
| | 0.35 | 1.20 | 1.21 | 1.32 |
| | 0.42 | 1.14 | 0.91 | 1.38 |
| | 0.25 | 1.22 | 0.52 | 1.25 |
| | 0.45 | 1.19 | 0.80 | 1.24 |
| | 0.48 | 1.22 | 1.36 | 1.37 |
| | 0.41 | 1.23 | 1.24 | 1.33 |
| | 0.27 | 1.23 | 1.28 | 1.23 |
| | 0.34 | 1.22 | 0.66 | 1.27 |
| | 0.37 | 1.20 | 1.12 | 1.26 |
| liver | 0.31 | 0.82 | 1.35 | 1.37 |
| | 0.42 | 0.85 | 1.20 | 1.43 |
| | 0.51 | 0.90 | 0.27 | 1.29 |
| | 0.25 | 0.80 | 0.25 | 1.32 |
| | 0.26 | 0.76 | 0.39 | 1.45 |
| | 0.30 | 0.79 | 0.15 | 1.34 |
| | 0.41 | 0.86 | 0.31 | 1.43 |
| | 0.25 | 0.87 | 1.43 | 1.36 |
| | 0.27 | 0.93 | 0.99 | 1.41 |
| | 0.35 | 0.91 | 1.10 | 1.31 |
| gall bladder | 0.44 | 0.85 | 0.94 | 1.21 |
| | 0.38 | 0.97 | 0.06 | 1.32 |
| | 0.39 | 0.96 | 0.61 | 1.23 |
| | 0.56 | 0.88 | 0.11 | 1.38 |
| | 0.36 | 0.99 | 0.07 | 1.35 |
| | 0.41 | 0.80 | 0.03 | 1.39 |
| | 0.39 | 0.87 | 0.15 | 1.29 |
| | 0.35 | 0.92 | 0.75 | 1.25 |
| | 0.50 | 1.00 | 0.68 | 1.16 |
| | 0.44 | 0.94 | 0.26 | 1.34 |
| kidney | 0.73 | 0.95 | 0.08 | 0.86 |
| | 0.80 | 0.89 | 0.66 | 1.01 |
| | 0.53 | 1.00 | 0.21 | 1.21 |
| | 0.57 | 0.87 | 1.29 | 0.92 |
| | 0.73 | 1.02 | 1.16 | 0.81 |
| | 0.61 | 1.04 | 0.75 | 0.87 |
| | 0.54 | 0.99 | 0.40 | 0.91 |
| | 0.46 | 0.90 | 0.26 | 0.86 |
| | 0.59 | 0.98 | 0.40 | 1.03 |
| | 0.57 | 1.07 | 0.88 | 0.79 |
| ureter | 0.96 | 1.13 | 0.42 | 1.16 |
| | 1.10 | 1.19 | 0.13 | 1.26 |
| | 0.90 | 0.90 | 0.66 | 1.21 |
| | 1.04 | 1.02 | 0.74 | 1.15 |
| | 1.01 | 0.85 | 0.25 | 1.32 |
| | 0.88 | 0.93 | 0.08 | 1.26 |
| | 1.16 | 1.00 | 0.54 | 1.27 |
| | 0.92 | 0.90 | 0.85 | 1.33 |
| | 1.13 | 1.10 | 0.13 | 1.22 |
| | 0.97 | 0.93 | 0.21 | 1.16 |
| sciatic nerve | 0.90 | 1.35 | 0.12 | 1.00 |
| | 0.92 | 1.37 | 0.07 | 1.17 |
| | 1.03 | 1.20 | 0.81 | 1.11 |
| | 0.99 | 1.51 | 0.45 | 1.15 |
| | 0.87 | 1.30 | 0.56 | 1.09 |
| | 0.91 | 1.25 | 0.60 | 1.03 |
| | 0.85 | 1.28 | 0.37 | 1.01 |
| | 0.87 | 1.37 | 0.31 | 1.22 |
| | 0.94 | 1.31 | 0.74 | 1.12 |
| | 0.84 | 1.29 | 0.19 | 1.14 |

TABLE B
Data relating to Example 1, summarized in Table 2 and FIG. 2

| | Tensile Strength (N) | | | |
|---|---|---|---|---|
| | Solder + | Cyanoacrylate + | Single 4-0 | Native |
| Tissue | Scaffold | Scaffold | Suture | Tissue |
| carotid artery | 0.83 | 1.04 | 1.06 | 0.95 |
| | 0.74 | 0.95 | 0.67 | 1.00 |
| | 0.80 | 1.07 | 0.64 | 0.92 |
| | 0.68 | 0.87 | 1.27 | 0.89 |
| | 0.80 | 1.00 | 0.55 | 0.86 |
| | 0.73 | 0.93 | 0.50 | 1.02 |
| | 0.70 | 1.04 | 0.41 | 0.90 |
| | 0.81 | 1.08 | 0.97 | 0.88 |
| | 0.77 | 0.99 | 1.21 | 0.87 |
| | 0.75 | 1.10 | 1.27 | 0.91 |
| femoral artery | 0.84 | 0.99 | 0.44 | 1.01 |
| | 0.76 | 0.94 | 0.96 | 1.05 |
| | 0.80 | 1.02 | 0.86 | 1.00 |
| | 0.73 | 1.06 | 0.34 | 1.11 |
| | 0.83 | 0.97 | 0.71 | 1.04 |
| | 0.80 | 1.00 | 0.65 | 0.98 |
| | 0.74 | 1.07 | 0.91 | 1.03 |
| | 0.77 | 1.10 | 1.02 | 1.00 |
| | 0.82 | 1.03 | 0.85 | 1.02 |
| | 0.77 | 1.00 | 0.49 | 1.00 |
| splenic artery | 1.02 | 1.43 | 1.29 | 1.53 |
| | 1.02 | 1.48 | 0.61 | 1.48 |
| | 1.08 | 1.34 | 0.47 | 1.51 |
| | 1.04 | 1.31 | 1.38 | 1.45 |
| | 1.14 | 1.45 | 1.51 | 1.54 |
| | 1.09 | 1.36 | 1.33 | 1.49 |
| | 0.97 | 1.39 | 0.35 | 1.55 |
| | 1.12 | 1.34 | 0.63 | 1.41 |
| | 1.03 | 1.47 | 0.74 | 1.45 |
| | 0.90 | 1.46 | 0.85 | 1.47 |
| coronary artery | 0.92 | 1.29 | 0.96 | 1.49 |
| | 1.01 | 1.46 | 1.47 | 1.60 |
| | 1.06 | 1.35 | 1.12 | 1.56 |
| | 0.99 | 1.32 | 1.16 | 1.55 |
| | 0.94 | 1.39 | 1.23 | 1.66 |
| | 0.97 | 1.44 | 1.43 | 1.50 |
| | 1.11 | 1.46 | 0.74 | 1.54 |
| | 1.05 | 1.43 | 0.90 | 1.51 |
| | 1.09 | 1.30 | 1.33 | 1.58 |
| | 0.92 | 1.44 | 1.40 | 1.49 |
| pulmonary artery | 0.93 | 1.38 | 0.99 | 1.59 |
| | 1.06 | 1.22 | 0.75 | 1.43 |
| | 1.03 | 1.40 | 1.18 | 1.55 |
| | 0.79 | 1.44 | 0.61 | 1.49 |
| | 0.86 | 1.35 | 0.97 | 1.40 |
| | 0.93 | 1.33 | 0.51 | 1.45 |
| | 0.91 | 1.39 | 0.67 | 1.52 |
| | 0.88 | 1.23 | 1.02 | 1.54 |
| | 1.02 | 1.32 | 0.87 | 1.59 |
| | 0.99 | 1.30 | 1.14 | 1.61 |
| aorta (intima) | 1.12 | 1.59 | 1.40 | 1.64 |
| | 1.00 | 1.47 | 0.69 | 1.60 |
| | 1.25 | 1.33 | 1.24 | 1.66 |
| | 0.92 | 1.64 | 0.87 | 1.59 |
| | 1.06 | 1.44 | 1.36 | 1.60 |
| | 0.97 | 1.39 | 1.56 | 1.55 |
| | 1.22 | 1.50 | 0.60 | 1.51 |
| | 1.08 | 1.43 | 0.46 | 1.56 |
| | 1.02 | 1.56 | 1.11 | 1.64 |
| | 1.14 | 1.50 | 1.43 | 1.55 |
| aorta (adventitia) | 1.20 | 1.29 | 1.03 | 1.59 |
| | 1.23 | 1.42 | 1.29 | 1.50 |
| | 1.08 | 1.44 | 1.38 | 1.54 |
| | 1.29 | 1.36 | 1.23 | 1.65 |
| | 1.33 | 1.33 | 1.21 | 1.60 |
| | 1.35 | 1.39 | 1.19 | 1.66 |
| | 1.26 | 1.44 | 1.32 | 1.51 |
| | 1.00 | 1.50 | 0.95 | 1.56 |
| | 1.23 | 1.46 | 1.44 | 1.48 |
| | 1.40 | 1.54 | 0.87 | 1.57 |

TABLE C
Data relating to Example 3, summarized in FIG. 6

| | Tensile Strength (N) | | | |
|---|---|---|---|---|
| | | Cyanoacrylate | Cyanoacrylate + | Cyanoacrylate + |
| Rat | Suture | Alone | SIS | PLGA |
| 1 | 6.2 | 3.5 | 8.5 | 6.8 |
| 2 | 4.7 | 5.3 | 7.0 | 7.8 |
| 3 | 6.3 | 3.7 | 8.0 | 6.3 |
| 4 | 2.5 | 5.8 | 5.5 | 8.1 |
| 5 | 2.0 | 6.5 | 9.0 | 8.1 |
| 6 | 4.5 | 4.9 | 8.4 | 7.2 |
| 7 | 5.2 | 3.2 | 6.6 | 8.6 |
| 8 | 2.4 | 5.0 | 6.2 | 6.7 |
| 9 | 6.2 | 5.9 | 9.1 | 7.1 |
| 10 | 5.5 | 4.3 | 7.0 | 6.5 |
| Mean | 4.3 | 5.0 | 7.6 | 7.4 |
| St Dev | 1.6 | 1.0 | 1.2 | 0.8 |

TABLE D
Data relating to Example 3, summarized in FIG. 7

| | Time to Failure (s) | | | |
|---|---|---|---|---|
| | | Cyanoacrylate | Cyanoacrylate + | Cyanoacrylate + |
| Rat | Suture | Alone | SIS | PLGA |
| 1 | 40 | 65 | 160 | 125 |
| 2 | 55 | 40 | 150 | 150 |
| 3 | 95 | 30 | 75 | 55 |
| 4 | 65 | 70 | 90 | 110 |
| 5 | 60 | 85 | 85 | 100 |
| 6 | 60 | 60 | 95 | 120 |
| 7 | 55 | 45 | 105 | 135 |
| 8 | 45 | 50 | 80 | 80 |
| 9 | 70 | 75 | 140 | 115 |
| 10 | 65 | 60 | 135 | 95 |
| Mean | 61 | 58 | 112 | 108 |
| St Dev | 13 | 17 | 27 | 27 |

TABLE E
Data relating to Example 3, summarized in FIG. 9

| | Tensile Strength (N) | | | |
|---|---|---|---|---|
| | | Cyanoacrylate | Cyanoacrylate + | Cyanoacrylate + |
| Rat | Suture | Alone | SIS | PLGA |
| 1 | 7.0 | 5.6 | 8.0 | 8.9 |
| 2 | 6.5 | 6.3 | 9.2 | 8.7 |
| 3 | 4.2 | 4.8 | 8.3 | 7.9 |
| Mean | 5.9 | 5.6 | 8.5 | 8.5 |
| St Dev | 1.2 | 0.8 | 0.5 | 0.4 |

TABLE F
Data relating to Example 3, summarized in FIG. 10

| | Time to Failure (s) | | | |
|---|---|---|---|---|
| | | Cyanoacrylate | Cyanoacrylate + | Cyanoacrylate + |
| Rat | Suture | Alone | SIS | PLGA |
| 1 | 54 | 56 | 128 | 136 |
| 2 | 73 | 69 | 143 | 152 |
| 3 | 88 | 60 | 125 | 127 |
| Mean | 72 | 62 | 132 | 138 |
| St Dev | 9 | 5 | 9 | 13 |

TABLE G
Data relating to Example 4, summarized in FIG. 11

| | Tensile Strength (N) | | |
|---|---|---|---|
| | Cyanoacrylate | Cyanoacrylate + | Cyanoacrylate + |
| Specimen | alone | SIS | PLGA |
| 1 | 1.34 | 3.32 | 2.64 |
| 2 | 2.55 | 2.20 | 2.05 |
| 3 | 0.71 | 2.77 | 2.28 |
| 4 | 0.89 | 1.83 | 2.09 |
| 5 | 1.15 | 1.77 | 2.17 |
| 6 | 0.72 | 2.27 | 1.63 |
| 7 | 1.42 | 2.10 | 2.94 |
| 8 | 1.79 | 2.32 | 2.62 |
| 9 | 1.80 | 1.99 | 2.29 |
| 10 | 1.54 | 2.45 | 2.19 |
| Mean | 1.39 | 2.30 | 2.29 |
| St Dev | 0.57 | 0.46 | 0.37 |

TABLE H
Data relating to Example 4, summarized in FIG. 12

| | Tensile | | Tensile | | Tensile |
|---|---|---|---|---|---|
| | Strength (N) − | | Strength (N) − | | Strength (N) − |
| | Cyanoacrylate | | Cyanoacrylate + | | Cyanoacrylate + |
| Time (s) | Alone | Time (s) | SIS | Time (s) | PLGA |
| 0.5662 | −0.0091 | 0.5254 | 0.0565 | 0.6558 | 0.0635 |
| 1.0662 | 0.0111 | 1.0254 | 0.0930 | 1.1558 | −0.0600 |
| 1.5662 | 0.0272 | 1.5254 | −0.0260 | 1.6558 | 0.0025 |
| 2.0662 | −0.0024 | 2.0254 | 0.0490 | 2.1558 | 0.0786 |
| 2.5662 | −0.0013 | 2.5254 | 0.0945 | 2.6558 | −0.0486 |
| 3.0662 | 0.0257 | 3.0254 | −0.0230 | 3.1558 | 0.0026 |
| 3.5662 | −0.0057 | 3.5254 | 0.0544 | 3.6558 | 0.0646 |
| 4.0662 | 0.0008 | 4.0254 | 0.0890 | 4.1558 | −0.0443 |
| 4.5662 | 0.0236 | 4.5254 | −0.0171 | 4.6558 | 0.0063 |
| 5.0662 | −0.0095 | 5.0254 | 0.0564 | 5.1558 | 0.0693 |
| 5.5662 | 0.0034 | 5.5254 | 0.0873 | 5.6558 | −0.0490 |
| 6.0662 | 0.0258 | 6.0254 | −0.0227 | 6.1558 | 0.0108 |
| 6.5662 | −0.0096 | 6.5254 | 0.0596 | 6.6558 | 0.0736 |
| 7.0662 | 0.0076 | 7.0254 | 0.0849 | 7.1558 | −0.0493 |
| 7.5662 | 0.0195 | 7.5254 | −0.0290 | 7.6558 | 0.0167 |
| 8.0662 | −0.0054 | 8.0254 | 0.0525 | 8.1558 | 0.0673 |
| 8.5662 | 0.0129 | 8.5254 | 0.0892 | 8.6558 | −0.0494 |
| 9.0662 | 0.0213 | 9.0254 | −0.0281 | 9.1558 | 0.0114 |
| 9.5662 | 0.0003 | 9.5254 | 0.0678 | 9.6558 | 0.0641 |
| 10.0662 | 0.0110 | 10.0254 | 0.0888 | 10.1558 | −0.0497 |
| 10.5662 | 0.0207 | 10.5254 | −0.0282 | 10.6558 | 0.0018 |
| 11.0662 | −0.0086 | 11.0254 | 0.0645 | 11.1558 | 0.0716 |
| 11.5662 | 0.0070 | 11.5254 | 0.0869 | 11.6558 | −0.0540 |
| 12.0662 | 0.0194 | 12.0254 | −0.0324 | 12.1558 | 0.0043 |
| 12.5662 | −0.0093 | 12.5254 | 0.0650 | 12.6558 | 0.0608 |
| 13.0662 | 0.0026 | 13.0254 | 0.0844 | 13.1558 | −0.0581 |
| 13.5662 | 0.0245 | 13.5254 | −0.0252 | 13.6558 | 0.0261 |
| 14.0662 | 0.0021 | 14.0254 | 0.0553 | 14.1558 | 0.0598 |
| 14.5662 | 0.0033 | 14.5254 | 0.0890 | 14.6558 | −0.0586 |
| 15.0662 | 0.0201 | 15.0254 | −0.0227 | 15.1558 | 0.0074 |
| 15.5662 | −0.0061 | 15.5254 | 0.0691 | 15.6558 | 0.0670 |
| 16.0662 | 0.0071 | 16.0254 | 0.0854 | 16.1558 | −0.0553 |
| 16.5662 | 0.0207 | 16.5254 | −0.0301 | 16.6558 | 0.0227 |
| 17.0662 | −0.0085 | 17.0254 | 0.0672 | 17.1558 | 0.0690 |
| 17.5662 | 0.0111 | 17.5254 | 0.0922 | 17.6558 | −0.0505 |
| 18.0662 | 0.0245 | 18.0254 | −0.0173 | 18.1558 | 0.0126 |
| 18.5662 | −0.0057 | 18.5254 | 0.0709 | 18.6558 | 0.0719 |
| 19.0662 | 0.0115 | 19.0254 | 0.0899 | 19.1558 | −0.0388 |
| 19.5662 | 0.0241 | 19.5254 | −0.0284 | 19.6558 | 0.0217 |
| 20.0662 | −0.0024 | 20.0254 | 0.0709 | 20.1558 | 0.0671 |
| 20.5662 | 0.0107 | 20.5254 | 0.0825 | 20.6558 | −0.0414 |
| 21.0662 | 0.0235 | 21.0254 | −0.0286 | 21.1558 | 0.0370 |
| 21.5662 | −0.0032 | 21.5254 | 0.0662 | 21.6558 | 0.0711 |
| 22.0662 | 0.0077 | 22.0254 | 0.0896 | 22.1558 | −0.0435 |
| 22.5662 | 0.0235 | 22.5254 | −0.0217 | 22.6558 | 0.0466 |
| 23.0662 | −0.0041 | 23.0254 | 0.0746 | 23.1558 | 0.0701 |
| 23.5662 | 0.0089 | 23.5254 | 0.0835 | 23.6558 | −0.0435 |
| 24.0662 | 0.0243 | 24.0254 | −0.0274 | 24.1558 | 0.0437 |
| 24.5662 | 0.0064 | 24.5254 | 0.0753 | 24.6558 | 0.0747 |
| 25.0662 | 0.0130 | 25.0254 | 0.0860 | 25.1558 | −0.0515 |
| 25.5662 | 0.0264 | 25.5254 | −0.0171 | 25.6558 | 0.0425 |
| 26.0662 | 0.0037 | 26.0254 | 0.0655 | 26.1558 | 0.0810 |
| 26.5662 | 0.0139 | 26.5254 | 0.0955 | 26.6558 | −0.0442 |
| 27.0662 | 0.0241 | 27.0254 | −0.0116 | 27.1558 | 0.0448 |
| 27.5662 | 0.0002 | 27.5254 | 0.0843 | 27.6558 | 0.0817 |
| 28.0662 | 0.0158 | 28.0254 | 0.0841 | 28.1558 | −0.0311 |
| 28.5662 | 0.0304 | 28.5254 | −0.0131 | 28.6558 | 0.0498 |
| 29.0662 | 0.0089 | 29.0254 | 0.0809 | 29.1558 | 0.0793 |
| 29.5662 | 0.0143 | 29.5254 | 0.0857 | 29.6558 | −0.0282 |
| 30.0662 | 0.0269 | 30.0254 | −0.0193 | 30.1558 | 0.0573 |
| 30.5662 | 0.0013 | 30.5254 | 0.0775 | 30.6558 | 0.0808 |
| 31.0662 | 0.0164 | 31.0254 | 0.0838 | 31.1558 | −0.0199 |
| 31.5662 | 0.0278 | 31.5254 | −0.0119 | 31.6558 | 0.0588 |
| 32.0662 | 0.0042 | 32.0254 | 0.0849 | 32.1558 | 0.0840 |
| 32.5662 | 0.0203 | 32.5254 | 0.0946 | 32.6558 | −0.0228 |
| 33.0662 | 0.0401 | 33.0254 | 0.0004 | 33.1558 | 0.0748 |
| 33.5662 | 0.0064 | 33.5254 | 0.0974 | 33.6558 | 0.0835 |
| 34.0662 | 0.0202 | 34.0254 | 0.0888 | 34.1558 | −0.0071 |
| 34.5662 | 0.0338 | 34.5254 | −0.0031 | 34.6558 | 0.0831 |
| 35.0662 | −0.0001 | 35.0254 | 0.0928 | 35.1558 | 0.1080 |
| 35.5662 | 0.0250 | 35.5254 | 0.1016 | 35.6558 | 0.0004 |
| 36.0662 | 0.0419 | 36.0254 | 0.0051 | 36.1558 | 0.0943 |
| 36.5662 | 0.0134 | 36.5254 | 0.1034 | 36.6558 | 0.1213 |
| 37.0662 | 0.0284 | 37.0254 | 0.0973 | 37.1558 | 0.0184 |
| 37.5662 | 0.0410 | 37.5254 | 0.0068 | 37.6558 | 0.1110 |
| 38.0662 | 0.0133 | 38.0254 | 0.1090 | 38.1558 | 0.1209 |
| 38.5662 | 0.0361 | 38.5254 | 0.1072 | 38.6558 | 0.0260 |
| 39.0662 | 0.0432 | 39.0254 | 0.0132 | 39.1558 | 0.1284 |
| 39.5662 | 0.0119 | 39.5254 | 0.1233 | 39.6558 | 0.1465 |
| 40.0662 | 0.0398 | 40.0254 | 0.1120 | 40.1558 | 0.0379 |
| 40.5662 | 0.0383 | 40.5254 | 0.0282 | 40.6558 | 0.1439 |
| 41.0662 | 0.0164 | 41.0254 | 0.1363 | 41.1558 | 0.1589 |
| 41.5662 | 0.0410 | 41.5254 | 0.1353 | 41.6558 | 0.0625 |
| 42.0662 | 0.0488 | 42.0254 | 0.0490 | 42.1558 | 0.1546 |
| 42.5662 | 0.0297 | 42.5254 | 0.1528 | 42.6558 | 0.1818 |
| 43.0662 | 0.0440 | 43.0254 | 0.1431 | 43.1558 | 0.0780 |
| 43.5662 | 0.0535 | 43.5254 | 0.0582 | 43.6558 | 0.1762 |
| 44.0662 | 0.0355 | 44.0254 | 0.1680 | 44.1558 | 0.2017 |
| 44.5662 | 0.0545 | 44.5254 | 0.1634 | 44.6558 | 0.0915 |
| 45.0662 | 0.0620 | 45.0254 | 0.0752 | 45.1558 | 0.2031 |
| 45.5662 | 0.0423 | 45.5254 | 0.1862 | 45.6558 | 0.2126 |
| 46.0662 | 0.0726 | 46.0254 | 0.1739 | 46.1558 | 0.1311 |
| 46.5662 | 0.0801 | 46.5254 | 0.0900 | 46.6558 | 0.2274 |
| 47.0662 | 0.0516 | 47.0254 | 0.2173 | 47.1558 | 0.2379 |
| 47.5662 | 0.0822 | 47.5254 | 0.1934 | 47.6558 | 0.1582 |
| 48.0662 | 0.0904 | 48.0254 | 0.1243 | 48.1558 | 0.2486 |
| 48.5662 | 0.0598 | 48.5254 | 0.2412 | 48.6558 | 0.2522 |
| 49.0662 | 0.0873 | 49.0254 | 0.2264 | 49.1558 | 0.1682 |
| 49.5662 | 0.1030 | 49.5254 | 0.1412 | 49.6558 | 0.2759 |
| 50.0662 | 0.0784 | 50.0254 | 0.2571 | 50.1558 | 0.2871 |
| 50.5662 | 0.1137 | 50.5254 | 0.2453 | 50.6558 | 0.1933 |
| 51.0662 | 0.1229 | 51.0254 | 0.1610 | 51.1558 | 0.2939 |
| 51.5662 | 0.1026 | 51.5254 | 0.3027 | 51.6558 | 0.3078 |
| 52.0662 | 0.1381 | 52.0254 | 0.2935 | 52.1558 | 0.2265 |
| 52.5662 | 0.1524 | 52.5254 | 0.2153 | 52.6558 | 0.3188 |
| 53.0662 | 0.1321 | 53.0254 | 0.3471 | 53.1558 | 0.3242 |
| 53.5662 | 0.1725 | 53.5254 | 0.3322 | 53.6558 | 0.2489 |
| 54.0662 | 0.1852 | 54.0254 | 0.2531 | 54.1558 | 0.3463 |
| 54.5662 | 0.1705 | 54.5254 | 0.3860 | 54.6558 | 0.3477 |
| 55.0662 | 0.2095 | 55.0254 | 0.3812 | 55.1558 | 0.2729 |
| 55.5662 | 0.2245 | 55.5254 | 0.3050 | 55.6558 | 0.3698 |
| 56.0662 | 0.2080 | 56.0254 | 0.4264 | 56.1558 | 0.3797 |
| 56.5662 | 0.2470 | 56.5254 | 0.4006 | 56.6558 | 0.3092 |
| 57.0662 | 0.2689 | 57.0254 | 0.3451 | 57.1558 | 0.3952 |
| 57.5662 | 0.2602 | 57.5254 | 0.4732 | 57.6558 | 0.3968 |
| 58.0662 | 0.3262 | 58.0254 | 0.4389 | 58.1558 | 0.3451 |
| 58.5662 | 0.3497 | 58.5254 | 0.3866 | 58.6558 | 0.4444 |
| 59.0662 | 0.3354 | 59.0254 | 0.5191 | 59.1558 | 0.4252 |
| 59.5662 | 0.3799 | 59.5254 | 0.5147 | 59.6558 | 0.3365 |
| 60.0662 | 0.4002 | 60.0254 | 0.4365 | 60.1558 | 0.4531 |
| 60.5662 | 0.4047 | 60.5254 | 0.5677 | 60.6558 | 0.4646 |
| 61.0662 | 0.4626 | 61.0254 | 0.5392 | 61.1558 | 0.3788 |
| 61.5662 | 0.4843 | 61.5254 | 0.4821 | 61.6558 | 0.5118 |
| 62.0662 | 0.4841 | 62.0254 | 0.6078 | 62.1558 | 0.5079 |
| 62.5662 | 0.5096 | 62.5254 | 0.5869 | 62.6558 | 0.4284 |
| 63.0662 | 0.5346 | 63.0254 | 0.5103 | 63.1558 | 0.5610 |
| 63.5662 | 0.5401 | 63.5254 | 0.6340 | 63.6558 | 0.5560 |
| 64.0662 | 0.6048 | 64.0254 | 0.6051 | 64.1558 | 0.4850 |
| 64.5662 | 0.6434 | 64.5254 | 0.5433 | 64.6558 | 0.6229 |
| 65.0662 | 0.6466 | 65.0254 | 0.6627 | 65.1558 | 0.5912 |
| 65.5662 | 0.7034 | 65.5254 | 0.6489 | 65.6558 | 0.5206 |
| 66.0662 | 0.7319 | 66.0254 | 0.5903 | 66.1558 | 0.6515 |
| 66.5662 | 0.7296 | 66.5254 | 0.7124 | 66.6558 | 0.6560 |
| 67.0662 | 0.7812 | 67.0254 | 0.6868 | 67.1558 | 0.5663 |
| 67.5662 | 0.8245 | 67.5254 | 0.6226 | 67.6558 | 0.6745 |
| 68.0662 | 0.8143 | 68.0254 | 0.7311 | 68.1558 | 0.6949 |
| 68.5662 | 0.8697 | 68.5254 | 0.6968 | 68.6558 | 0.5977 |
| 69.0662 | 0.8950 | 69.0254 | 0.6694 | 69.1558 | 0.7006 |
| 69.5662 | 0.8903 | 69.5254 | 0.7460 | 69.6558 | 0.7167 |
| 70.0662 | 0.9447 | 70.0254 | 0.7182 | 70.1558 | 0.6466 |
| 70.5662 | 0.9580 | 70.5254 | 0.6671 | 70.6558 | 0.7853 |
| 71.0662 | 0.9722 | 71.0254 | 0.7869 | 71.1558 | 0.7667 |
| 71.5662 | 1.0043 | 71.5254 | 0.7528 | 71.6558 | 0.6706 |
| 72.0662 | 1.0323 | 72.0254 | 0.7061 | 72.1558 | 0.7699 |
| 72.5662 | 1.0003 | 72.5254 | 0.8182 | 72.6558 | 0.7282 |
| 73.0662 | 1.0034 | 73.0254 | 0.7819 | 73.1558 | 0.6681 |
| 73.5662 | 1.0202 | 73.5254 | 0.7247 | 73.6558 | 0.7883 |
| 74.0662 | 0.9740 | 74.0254 | 0.8305 | 74.1558 | 0.7932 |
| 74.5662 | 0.9786 | 74.5254 | 0.7982 | 74.6558 | 0.7392 |
| 75.0662 | 0.9916 | 75.0254 | 0.7542 | 75.1558 | 0.8474 |
| 75.5662 | 0.9799 | 75.5254 | 0.8685 | 75.6558 | 0.8278 |
| 76.0662 | 0.9983 | 76.0254 | 0.8127 | 76.1558 | 0.7282 |
| 76.5662 | 0.9970 | 76.5254 | 0.7563 | 76.6558 | 0.8575 |
| 77.0662 | 0.9838 | 77.0254 | 0.8622 | 77.1558 | 0.7959 |
| 77.5662 | 0.9859 | 77.5254 | 0.8337 | 77.6558 | 0.7446 |
| 78.0662 | 0.9690 | 78.0254 | 0.7886 | 78.1558 | 0.8550 |
| 78.5662 | 0.9315 | 78.5254 | 0.9194 | 78.6558 | 0.8127 |
| 79.0662 | 0.9622 | 79.0254 | 0.8416 | 79.1558 | 0.7529 |
| 79.5662 | 0.9899 | 79.5254 | 0.8144 | 79.6558 | 0.8798 |
| 80.0662 | 0.9726 | 80.0254 | 0.9137 | 80.1558 | 0.8644 |
| 80.5662 | 0.9978 | 80.5254 | 0.8873 | 80.6558 | 0.8303 |
| 81.0662 | 0.9357 | 81.0254 | 0.8639 | 81.1558 | 0.9743 |
| 81.5662 | 0.8981 | 81.5254 | 0.9849 | 81.6558 | 0.9309 |
| 82.0662 | 0.8623 | 82.0254 | 0.9298 | 82.1558 | 0.8491 |
| 82.5662 | 0.7964 | 82.5254 | 0.9306 | 82.6558 | 0.9989 |
| 83.0662 | 0.7561 | 83.0254 | 1.0418 | 83.1558 | 0.9567 |
| 83.5662 | 0.7497 | 83.5254 | 0.9784 | 83.6558 | 0.9116 |
| 84.0662 | 0.7104 | 84.0254 | 0.9776 | 84.1558 | 1.0292 |
| 84.5662 | 0.6552 | 84.5254 | 1.0905 | 84.6558 | 1.0169 |
| 85.0662 | 0.6317 | 85.0254 | 1.0160 | 85.1558 | 0.9501 |
| 85.5662 | 0.4368 | 85.5254 | 1.0025 | 85.6558 | 1.0932 |
| 86.0662 | 0.4109 | 86.0254 | 1.0888 | 86.1558 | 1.0846 |
| 86.5662 | 0.4253 | 86.5254 | 0.9953 | 86.6558 | 1.0228 |
| 87.0662 | 0.4164 | 87.0254 | 0.9726 | 87.1558 | 1.1636 |
| 87.5662 | 0.3940 | 87.5254 | 1.0686 | 87.6558 | 1.1369 |
| 88.0662 | 0.3914 | 88.0254 | 0.9988 | 88.1558 | 1.0708 |
| 88.5662 | 0.1006 | 88.5254 | 0.9735 | 88.6558 | 1.2250 |
| 89.0662 | 0.0791 | 89.0254 | 1.1150 | 89.1558 | 1.2148 |
| 89.5662 | 0.1035 | 89.5254 | 1.0385 | 89.6558 | 1.1490 |
| 90.0662 | 0.0934 | 90.0254 | 1.0114 | 90.1558 | 1.3004 |
| 90.5662 | 0.0609 | 90.5254 | 1.1318 | 90.6558 | 1.2924 |
| 91.0662 | 0.0901 | 91.0254 | 1.0501 | 91.1558 | 1.2294 |
| 91.5662 | 0.0795 | 91.5254 | 1.0027 | 91.6558 | 1.3589 |
| 92.0662 | 0.0477 | 92.0254 | 1.1004 | 92.1558 | 1.3312 |
| 92.5662 | 0.0818 | 92.5254 | 1.0152 | 92.6558 | 1.2635 |
| 93.0662 | 0.0717 | 93.0254 | 0.9960 | 93.1558 | 1.3822 |
| 93.5662 | 0.0554 | 93.5254 | 1.1032 | 93.6558 | 1.3359 |
| 94.0662 | 0.0753 | 94.0254 | 1.0304 | 94.1558 | 1.2581 |
| 94.5662 | 0.0754 | 94.5254 | 0.9952 | 94.6558 | 1.3693 |
| 95.0662 | 0.0525 | 95.0254 | 1.0993 | 95.1558 | 1.3083 |
| 95.5662 | 0.0433 | 95.5254 | 1.0268 | 95.6558 | 1.2484 |
| 96.0662 | 0.0216 | 96.0254 | 1.0040 | 96.1558 | 1.3356 |
| 96.5662 | −0.0002 | 96.5254 | 1.1117 | 96.6558 | 1.2691 |
| 97.0662 | 0.0216 | 97.0254 | 1.0142 | 97.1558 | 1.1935 |
| 97.5662 | 0.0167 | 97.5254 | 0.9717 | 97.6558 | 1.3079 |
| 98.0662 | 0.0071 | 98.0254 | 1.0963 | 98.1558 | 1.2440 |
| 98.5662 | 0.0238 | 98.5254 | 1.0002 | 98.6558 | 1.1641 |
| 99.0662 | 0.0128 | 99.0254 | 0.9735 | 99.1558 | 1.2714 |
| 99.5662 | 0.0116 | 99.5254 | 1.0762 | 99.6558 | 1.1953 |
| 100.0662 | 0.0328 | 100.0254 | 0.9837 | 100.1558 | 1.1641 |
| 100.5662 | 0.0255 | 100.5254 | 0.9627 | 100.6558 | 1.2371 |
| 101.0662 | 0.0198 | 101.0254 | 1.0702 | 101.1558 | 1.1685 |
| 101.5662 | 0.0189 | 101.5254 | 0.9871 | 101.6558 | 1.1014 |
| 102.0662 | 0.0067 | 102.0254 | 0.9567 | 102.1558 | 1.1958 |
| 102.5662 | −0.0121 | 102.5254 | 1.0603 | 102.6558 | 1.1316 |
| 103.0662 | 0.0127 | 103.0254 | 0.9650 | 103.1558 | 1.0533 |
| 103.5662 | 0.0058 | 103.5254 | 0.9274 | 103.6558 | 1.1714 |
| 104.0662 | 0.0004 | 104.0254 | 1.0448 | 104.1558 | 1.0882 |
| 104.5662 | 0.0123 | 104.5254 | 0.9637 | 104.6558 | 1.0081 |
| 105.0662 | 0.0035 | 105.0254 | 0.9281 | 105.1558 | 1.1301 |
| 105.5662 | −0.0105 | 105.5254 | 1.0257 | 105.6558 | 1.0427 |
| 106.0662 | 0.0165 | 106.0254 | 0.9495 | 106.1558 | 0.9803 |
| 106.5662 | 0.0021 | 106.5254 | 0.9351 | 106.6558 | 1.0825 |
| 107.0662 | −0.0057 | 107.0254 | 1.0271 | 107.1558 | 1.0183 |
| 107.5662 | 0.0173 | 107.5254 | 0.9369 | 107.6558 | 0.9506 |
| 108.0662 | 0.0054 | 108.0254 | 0.9235 | 108.1558 | 1.0779 |
| 108.5662 | −0.0061 | 108.5254 | 1.0173 | 108.6558 | 0.9794 |
| 109.0662 | 0.0160 | 109.0254 | 0.9315 | 109.1558 | 0.9463 |
| 109.5662 | 0.0031 | 109.5254 | 0.9225 | 109.6558 | 1.0473 |
| 110.0662 | −0.0055 | 110.0254 | 1.0246 | 110.1558 | 0.9756 |
| 110.5662 | 0.0073 | 110.5254 | 0.9408 | 110.6558 | 0.9154 |
| 111.0662 | 0.0007 | 111.0254 | 0.9238 | 111.1558 | 1.0425 |
| 111.5662 | −0.0063 | 111.5254 | 1.0217 | 111.6558 | 0.9925 |
| 112.0662 | 0.0151 | 112.0254 | 0.9648 | 112.1558 | 0.9371 |
| 112.5662 | 0.0048 | 112.5254 | 0.9459 | 112.6558 | 1.0297 |
| 113.0662 | −0.0111 | 113.0254 | 1.0614 | 113.1558 | 0.9714 |
| 113.5662 | 0.0142 | 113.5254 | 0.9684 | 113.6558 | 0.9519 |
| 114.0662 | 0.0033 | 114.0254 | 0.9527 | 114.1558 | 1.0538 |
| 114.5662 | −0.0074 | 114.5254 | 1.0556 | 114.6558 | 0.9820 |
| 115.0662 | 0.0169 | 115.0254 | 0.9729 | 115.1558 | 0.9400 |
| 115.5662 | 0.0059 | 115.5254 | 0.9484 | 115.6558 | 1.0619 |
| 116.0662 | −0.0094 | 116.0254 | 1.0482 | 116.1558 | 0.9704 |
| 116.5662 | 0.0154 | 116.5254 | 0.9606 | 116.6558 | 0.9587 |
| 117.0662 | 0.0007 | 117.0254 | 0.9581 | 117.1558 | 1.0770 |
| 117.5662 | −0.0078 | 117.5254 | 1.0623 | 117.6558 | 0.9903 |
| 118.0662 | 0.0149 | 118.0254 | 0.9700 | 118.1558 | 0.9483 |
| 118.5662 | −0.0019 | 118.5254 | 0.9716 | 118.6558 | 1.0798 |
| 119.0662 | −0.0056 | 119.0254 | 1.0570 | 119.1558 | 0.9804 |
| 119.5662 | 0.0209 | 119.5254 | 0.9815 | 119.6558 | 0.9520 |
| 120.0662 | 0.0004 | 120.0254 | 0.9812 | 120.1558 | 1.0850 |
| 120.5662 | −0.0097 | 120.5254 | 1.0638 | 120.6558 | 0.9978 |
| 121.0662 | 0.0124 | 121.0254 | 0.9834 | 121.1558 | 0.9458 |
| 121.5662 | 0.0052 | 121.5254 | 0.9871 | 121.6558 | 1.0709 |
| 122.0662 | −0.0065 | 122.0254 | 1.0778 | 122.1558 | 0.9864 |
| 122.5662 | 0.0148 | 122.5254 | 0.9988 | 122.6558 | 0.9773 |
| 123.0662 | 0.0003 | 123.0254 | 0.9992 | 123.1558 | 1.0836 |
| 123.5662 | −0.0092 | 123.5254 | 1.0677 | 123.6558 | 1.0040 |
| 124.0662 | 0.0194 | 124.0254 | 1.0043 | 124.1558 | 0.9886 |
| 124.5662 | −0.0035 | 124.5254 | 1.0137 | 124.6558 | 1.1052 |
| 125.0662 | −0.0098 | 125.0254 | 1.1097 | 125.1558 | 1.0077 |
| 125.5662 | 0.0151 | 125.5254 | 1.0558 | 125.6558 | 1.0127 |
| 126.0662 | 0.0010 | 126.0254 | 1.0592 | 126.1558 | 1.1126 |
| 126.5662 | −0.0044 | 126.5254 | 1.1658 | 126.6558 | 1.0206 |
| 127.0662 | 0.0183 | 127.0254 | 1.0812 | 127.1558 | 1.0242 |
| 127.5662 | −0.0001 | 127.5254 | 1.0895 | 127.6558 | 1.1280 |
| 128.0662 | −0.0022 | 128.0254 | 1.1711 | 128.1558 | 1.0523 |
| 128.5662 | 0.0120 | 128.5254 | 1.0802 | 128.6558 | 1.0318 |
| | | 129.0254 | 1.1171 | 129.1558 | 1.1481 |
| | | 129.5254 | 1.2157 | 129.6558 | 1.0756 |
| | | 130.0254 | 1.1282 | 130.1558 | 1.0429 |
| | | 130.5254 | 1.1378 | 130.6558 | 1.1728 |
| | | 131.0254 | 1.2270 | 131.1558 | 1.1039 |
| | | 131.5254 | 1.1425 | 131.6558 | 1.0691 |
| | | 132.0254 | 1.1632 | 132.1558 | 1.1731 |
| | | 132.5254 | 1.2470 | 132.6558 | 1.0991 |
| | | 133.0254 | 1.1461 | 133.1558 | 1.0742 |
| | | 133.5254 | 1.1660 | 133.6558 | 1.2086 |
| | | 134.0254 | 1.2594 | 134.1558 | 1.0867 |
| | | 134.5254 | 1.1567 | 134.6558 | 1.0952 |
| | | 135.0254 | 1.1904 | 135.1558 | 1.2207 |
| | | 135.5254 | 1.2565 | 135.6558 | 1.1233 |
| | | 136.0254 | 1.1689 | 136.1558 | 1.1081 |
| | | 136.5254 | 1.1970 | 136.6558 | 1.2359 |
| | | 137.0254 | 1.2888 | 137.1558 | 1.1756 |
| | | 137.5254 | 1.1667 | 137.6558 | 1.1398 |
| | | 138.0254 | 1.2043 | 138.1558 | 1.2702 |
| | | 138.5254 | 1.2745 | 138.6558 | 1.1769 |
| | | 139.0254 | 1.1801 | 139.1558 | 1.1765 |
| | | 139.5254 | 1.1982 | 139.6558 | 1.2667 |
| | | 140.0254 | 1.2596 | 140.1558 | 1.1985 |
| | | 140.5254 | 1.1590 | 140.6558 | 1.1905 |
| | | 141.0254 | 1.2049 | 141.1558 | 1.2736 |
| | | 141.5254 | 1.2720 | 141.6558 | 1.1891 |
| | | 142.0254 | 1.1880 | 142.1558 | 1.1953 |
| | | 142.5254 | 1.2258 | 142.6558 | 1.2958 |
| | | 143.0254 | 1.3077 | 143.1558 | 1.1958 |
| | | 143.5254 | 1.2122 | 143.6558 | 1.1882 |
| | | 144.0254 | 1.2288 | 144.1558 | 1.3123 |
| | | 144.5254 | 1.3213 | 144.6558 | 1.2290 |
| | | 145.0254 | 1.2364 | 145.1558 | 1.2235 |
| | | 145.5254 | 1.2691 | 145.6558 | 1.3367 |
| | | 146.0254 | 1.3469 | 146.1558 | 1.2463 |
| | | 146.5254 | 1.2800 | 146.6558 | 1.2479 |
| | | 147.0254 | 1.3131 | 147.1558 | 1.3480 |
| | | 147.5254 | 1.3980 | 147.6558 | 1.2488 |
| | | 148.0254 | 1.2787 | 148.1558 | 1.2773 |
| | | 148.5254 | 1.3219 | 148.6558 | 1.3719 |
| | | 149.0254 | 1.4038 | 149.1558 | 1.2705 |
| | | 149.5254 | 1.3058 | 149.6558 | 1.2849 |
| | | 150.0254 | 1.3435 | 150.1558 | 1.3786 |
| | | 150.5254 | 1.4282 | 150.6558 | 1.2698 |
| | | 151.0254 | 1.3252 | 151.1558 | 1.2759 |
| | | 151.5254 | 1.3688 | 151.6558 | 1.3885 |
| | | 152.0254 | 1.4159 | 152.1558 | 1.2786 |
| | | 152.5254 | 1.3090 | 152.6558 | 1.2968 |
| | | 153.0254 | 1.3378 | 153.1558 | 1.3819 |
| | | 153.5254 | 1.4103 | 153.6558 | 1.2831 |
| | | 154.0254 | 1.2855 | 154.1558 | 1.3039 |
| | | 154.5254 | 1.3340 | 154.6558 | 1.4115 |
| | | 155.0254 | 1.4151 | 155.1558 | 1.2880 |
| | | 155.5254 | 1.3076 | 155.6558 | 1.3021 |
| | | 156.0254 | 1.3545 | 156.1558 | 1.4125 |
| | | 156.5254 | 1.4471 | 156.6558 | 1.3162 |
| | | 157.0254 | 1.3450 | 157.1558 | 1.3283 |
| | | 157.5254 | 1.4047 | 157.6558 | 1.4228 |
| | | 158.0254 | 1.4631 | 158.1558 | 1.3359 |
| | | 158.5254 | 1.3631 | 158.6558 | 1.3380 |
| | | 159.0254 | 1.4150 | 159.1558 | 1.4404 |
| | | 159.5254 | 1.4740 | 159.6558 | 1.3516 |
| | | 160.0254 | 1.3650 | 160.1558 | 1.3666 |
| | | 160.5254 | 1.3987 | 160.6558 | 1.4505 |
| | | 161.0254 | 1.4800 | 161.1558 | 1.3330 |
| | | 161.5254 | 1.3543 | 161.6558 | 1.3832 |
| | | 162.0254 | 1.3875 | 162.1558 | 1.4469 |
| | | 162.5254 | 1.4435 | 162.6558 | 1.3547 |
| | | 163.0254 | 1.3313 | 163.1558 | 1.3792 |
| | | 163.5254 | 1.3386 | 163.6558 | 1.4480 |
| | | 164.0254 | 1.3879 | 164.1558 | 1.3617 |
| | | 164.5254 | 1.2757 | 164.6558 | 1.3907 |
| | | 165.0254 | 1.3099 | 165.1558 | 1.5113 |
| | | 165.5254 | 1.3773 | 165.6558 | 1.4053 |
| | | 166.0254 | 1.2621 | 166.1558 | 1.4040 |
| | | 166.5254 | 1.3001 | 166.6558 | 1.5089 |
| | | 167.0254 | 1.3716 | 167.1558 | 1.4075 |
| | | 167.5254 | 1.2621 | 167.6558 | 1.4319 |
| | | 168.0254 | 1.3177 | 168.1558 | 1.5345 |
| | | 168.5254 | 1.3990 | 168.6558 | 1.4371 |
| | | 169.0254 | 1.3097 | 169.1558 | 1.4636 |
| | | 169.5254 | 1.3492 | 169.6558 | 1.5517 |
| | | 170.0254 | 1.4388 | 170.1558 | 1.4529 |
| | | 170.5254 | 1.3552 | 170.6558 | 1.4708 |
| | | 171.0254 | 1.4291 | 171.1558 | 1.5767 |
| | | 171.5254 | 1.4757 | 171.6558 | 1.4623 |
| | | 172.0254 | 1.3934 | 172.1558 | 1.5162 |
| | | 172.5254 | 1.4594 | 172.6558 | 1.5952 |
| | | 173.0254 | 1.5054 | 173.1558 | 1.4814 |
| | | 173.5254 | 1.4260 | 173.6558 | 1.5150 |
| | | 174.0254 | 1.4967 | 174.1558 | 1.6018 |
| | | 174.5254 | 1.5615 | 174.6558 | 1.4697 |
| | | 175.0254 | 1.4691 | 175.1558 | 1.5124 |
| | | 175.5254 | 1.5155 | 175.6558 | 1.6151 |
| | | 176.0254 | 1.5755 | 176.1558 | 1.5009 |
| | | 176.5254 | 1.4786 | 176.6558 | 1.5527 |
| | | 177.0254 | 1.5610 | 177.1558 | 1.6275 |
| | | 177.5254 | 1.6044 | 177.6558 | 1.5192 |
| | | 178.0254 | 1.5219 | 178.1558 | 1.5530 |
| | | 178.5254 | 1.5954 | 178.6558 | 1.6408 |
| | | 179.0254 | 1.6321 | 179.1558 | 1.5225 |
| | | 179.5254 | 1.5354 | 179.6558 | 1.5530 |
| | | 180.0254 | 1.6005 | 180.1558 | 1.6510 |
| | | 180.5254 | 1.6334 | 180.6558 | 1.5523 |
| | | 181.0254 | 1.5508 | 181.1558 | 1.5947 |
| | | 181.5254 | 1.6327 | 181.6558 | 1.6629 |
| | | 182.0254 | 1.6559 | 182.1558 | 1.5604 |
| | | 182.5254 | 1.5468 | 182.6558 | 1.5933 |
| | | 183.0254 | 1.6326 | 183.1558 | 1.6603 |
| | | 183.5254 | 1.6804 | 183.6558 | 1.5601 |
| | | 184.0254 | 1.5876 | 184.1558 | 1.5978 |
| | | 184.5254 | 1.6614 | 184.6558 | 1.6606 |
| | | 185.0254 | 1.6881 | 185.1558 | 1.5627 |
| | | 185.5254 | 1.5912 | 185.6558 | 1.6132 |
| | | 186.0254 | 1.6797 | 186.1558 | 1.6717 |
| | | 186.5254 | 1.7023 | 186.6558 | 1.5769 |
| | | 187.0254 | 1.5935 | 187.1558 | 1.6131 |
| | | 187.5254 | 1.6758 | 187.6558 | 1.6851 |
| | | 188.0254 | 1.7277 | 188.1558 | 1.5700 |
| | | 188.5254 | 1.6279 | 188.6558 | 1.6318 |
| | | 189.0254 | 1.7077 | 189.1558 | 1.6833 |
| | | 189.5254 | 1.7240 | 189.6558 | 1.5731 |
| | | 190.0254 | 1.6213 | 190.1558 | 1.6335 |
| | | 190.5254 | 1.7187 | 190.6558 | 1.6893 |
| | | 191.0254 | 1.7655 | 191.1558 | 1.5955 |
| | | 191.5254 | 1.6705 | 191.6558 | 1.6786 |
| | | 192.0254 | 1.7436 | 192.1558 | 1.7255 |
| | | 192.5254 | 1.7744 | 192.6558 | 1.6290 |
| | | 193.0254 | 1.6555 | 193.1558 | 1.6715 |
| | | 193.5254 | 1.7525 | 193.6558 | 1.7537 |
| | | 194.0254 | 1.7811 | 194.1558 | 1.6415 |
| | | 194.5254 | 1.6889 | 194.6558 | 1.6925 |
| | | 195.0254 | 1.7507 | 195.1558 | 1.7606 |
| | | 195.5254 | 1.7907 | 195.6558 | 1.6744 |
| | | 196.0254 | 1.7014 | 196.1558 | 1.7331 |
| | | 196.5254 | 1.7733 | 196.6558 | 1.7759 |
| | | 197.0254 | 1.8069 | 197.1558 | 1.6775 |
| | | 197.5254 | 1.7218 | 197.6558 | 1.7357 |
| | | 198.0254 | 1.8102 | 198.1558 | 1.8097 |
| | | 198.5254 | 1.8339 | 198.6558 | 1.6983 |
| | | 199.0254 | 1.7534 | 199.1558 | 1.7760 |
| | | 199.5254 | 1.8338 | 199.6558 | 1.8225 |
| | | 200.0254 | 1.8508 | 200.1558 | 1.7212 |
| | | 200.5254 | 1.7513 | 200.6558 | 1.7968 |
| | | 201.0254 | 1.8314 | 201.1558 | 1.8481 |
| | | 201.5254 | 1.8462 | 201.6558 | 1.7391 |
| | | 202.0254 | 1.7543 | 202.1558 | 1.7993 |
| | | 202.5254 | 1.8409 | 202.6558 | 1.8730 |
| | | 203.0254 | 1.8356 | 203.1558 | 1.7623 |
| | | 203.5254 | 1.7418 | 203.6558 | 1.8052 |
| | | 204.0254 | 1.8243 | 204.1558 | 1.8747 |
| | | 204.5254 | 1.8439 | 204.6558 | 1.7571 |
| | | 205.0254 | 1.7414 | 205.1558 | 1.8324 |
| | | 205.5254 | 1.8041 | 205.6558 | 1.8813 |
| | | 206.0254 | 1.8078 | 206.1558 | 1.7485 |
| | | 206.5254 | 1.7207 | 206.6558 | 1.8279 |
| | | 207.0254 | 1.8006 | 207.1558 | 1.8907 |
| | | 207.5254 | 1.7941 | 207.6558 | 1.7626 |
| | | 208.0254 | 1.6834 | 208.1558 | 1.8148 |
| | | 208.5254 | 1.7788 | 208.6558 | 1.8817 |
| | | 209.0254 | 1.7940 | 209.1558 | 1.7612 |
| | | 209.5254 | 1.7011 | 209.6558 | 1.8422 |
| | | 210.0254 | 1.8042 | 210.1558 | 1.8905 |
| | | 210.5254 | 1.8011 | 210.6558 | 1.7620 |
| | | 211.0254 | 1.7164 | 211.1558 | 1.8461 |
| | | 211.5254 | 1.8084 | 211.6558 | 1.8846 |
| | | 212.0254 | 1.8052 | 212.1558 | 1.7685 |
| | | 212.5254 | 1.7209 | 212.6558 | 1.8454 |
| | | 213.0254 | 1.8319 | 213.1558 | 1.9100 |
| | | 213.5254 | 1.8460 | 213.6558 | 1.7857 |
| | | 214.0254 | 1.7495 | 214.1558 | 1.8548 |
| | | 214.5254 | 1.8703 | 214.6558 | 1.9146 |
| | | 215.0254 | 1.8675 | 215.1558 | 1.8002 |
| | | 215.5254 | 1.7855 | 215.6558 | 1.8762 |
| | | 216.0254 | 1.9008 | 216.1558 | 1.9314 |
| | | 216.5254 | 1.8923 | 216.6558 | 1.8195 |
| | | 217.0254 | 1.8102 | 217.1558 | 1.9027 |
| | | 217.5254 | 1.9152 | 217.6558 | 1.9401 |
| | | 218.0254 | 1.9114 | 218.1558 | 1.8314 |
| | | 218.5254 | 1.8395 | 218.6558 | 1.9102 |
| | | 219.0254 | 1.9534 | 219.1558 | 1.9487 |
| | | 219.5254 | 1.9470 | 219.6558 | 1.8409 |
| | | 220.0254 | 1.8684 | 220.1558 | 1.9111 |
| | | 220.5254 | 1.9911 | 220.6558 | 1.9523 |
| | | 221.0254 | 1.9693 | 221.1558 | 1.8487 |
| | | 221.5254 | 1.8862 | 221.6558 | 1.9326 |
| | | 222.0254 | 1.9889 | 222.1558 | 1.9816 |
| | | 222.5254 | 1.9789 | 222.6558 | 1.8854 |
| | | 223.0254 | 1.8981 | 223.1558 | 1.9658 |
| | | 223.5254 | 2.0190 | 223.6558 | 1.9924 |
| | | 224.0254 | 2.0009 | 224.1558 | 1.8873 |
| | | 224.5254 | 1.9335 | 224.6558 | 1.9611 |
| | | 225.0254 | 2.0602 | 225.1558 | 2.0019 |
| | | 225.5254 | 2.0320 | 225.6558 | 1.8895 |
| | | 226.0254 | 1.9499 | 226.1558 | 1.9872 |
| | | 226.5254 | 2.0551 | 226.6558 | 2.0050 |
| | | 227.0254 | 2.0533 | 227.1558 | 1.8831 |
| | | 227.5254 | 1.9590 | 227.6558 | 1.9927 |
| | | 228.0254 | 2.0655 | 228.1558 | 2.0100 |
| | | 228.5254 | 2.0333 | 228.6558 | 1.9058 |
| | | 229.0254 | 1.9713 | 229.1558 | 2.0139 |
| | | 229.5254 | 2.0943 | 229.6558 | 2.0155 |
| | | 230.0254 | 2.0807 | 230.1558 | 1.9264 |
| | | 230.5254 | 2.0033 | 230.6558 | 2.0332 |
| | | 231.0254 | 2.1218 | 231.1558 | 2.0326 |
| | | 231.5254 | 2.0882 | 231.6558 | 1.9549 |
| | | 232.0254 | 2.0158 | 232.1558 | 2.0577 |
| | | 232.5254 | 2.1266 | 232.6558 | 2.0667 |
| | | 233.0254 | 2.1168 | 233.1558 | 1.9527 |
| | | 233.5254 | 2.0288 | 233.6558 | 2.0684 |
| | | 234.0254 | 2.1615 | 234.1558 | 2.1051 |
| | | 234.5254 | 2.1209 | 234.6558 | 1.9946 |
| | | 235.0254 | 2.0342 | 235.1558 | 2.0944 |
| | | 235.5254 | 2.1576 | 235.6558 | 2.1156 |
| | | 236.0254 | 2.1104 | 236.1558 | 2.0121 |
| | | 236.5254 | 2.0504 | 236.6558 | 2.1214 |
| | | 237.0254 | 2.1821 | 237.1558 | 2.1237 |
| | | 237.5254 | 2.1576 | 237.6558 | 2.0462 |
| | | 238.0254 | 2.0781 | 238.1558 | 2.1243 |
| | | 238.5254 | 2.2120 | 238.6558 | 2.1624 |
| | | 239.0254 | 2.1832 | 239.1558 | 2.0607 |
| | | 239.5254 | 2.0997 | 239.6558 | 2.1630 |
| | | 240.0254 | 2.2105 | 240.1558 | 2.1561 |
| | | 240.5254 | 2.1847 | 240.6558 | 2.0758 |
| | | 241.0254 | 2.0996 | 241.1558 | 2.1805 |
| | | 241.5254 | 2.2095 | 241.6558 | 2.1460 |
| | | 242.0254 | 2.1748 | 242.1558 | 2.0474 |
| | | 242.5254 | 2.1067 | 242.6558 | 2.1601 |
| | | 243.0254 | 2.2154 | 243.1558 | 2.1840 |
| | | 243.5254 | 2.1630 | 243.6558 | 2.0732 |
| | | 244.0254 | 2.1123 | 244.1558 | 2.1663 |
| | | 244.5254 | 2.2160 | 244.6558 | 2.1612 |
| | | 245.0254 | 2.1759 | 245.1558 | 2.0596 |
| | | 245.5254 | 2.1262 | 245.6558 | 2.1745 |
| | | 246.0254 | 2.2252 | 246.1558 | 2.1748 |
| | | 246.5254 | 2.1804 | 246.6558 | 2.0797 |
| | | 247.0254 | 2.1103 | 247.1558 | 2.1881 |
| | | 247.5254 | 2.2170 | 247.6558 | 2.1772 |
| | | 248.0254 | 2.1452 | 248.1558 | 2.0921 |
| | | 248.5254 | 2.0774 | 248.6558 | 2.2105 |
| | | 249.0254 | 2.1826 | 249.1558 | 2.2258 |
| | | 249.5254 | 2.1333 | 249.6558 | 2.1231 |
| | | 250.0254 | 2.0735 | 250.1558 | 2.2371 |
| | | 250.5254 | 2.1764 | 250.6558 | 2.2271 |
| | | 251.0254 | 2.1189 | 251.1558 | 2.1210 |
| | | 251.5254 | 2.0632 | 251.6558 | 2.2510 |
| | | 252.0254 | 2.1832 | 252.1558 | 2.2371 |
| | | 252.5254 | 2.0532 | 252.6558 | 2.1452 |
| | | 253.0254 | 1.9346 | 253.1558 | 2.2658 |
| | | 253.5254 | 2.0401 | 253.6558 | 2.2824 |
| | | 254.0254 | 1.9773 | 254.1558 | 2.1720 |
| | | 254.5254 | 1.8949 | 254.6558 | 2.2722 |
| | | 255.0254 | 2.0064 | 255.1558 | 2.2611 |
| | | 255.5254 | 1.9560 | 255.6558 | 2.1583 |
| | | 256.0254 | 1.8667 | 256.1558 | 2.2602 |
| | | 256.5254 | 2.0311 | 256.6558 | 2.2697 |
| | | 257.0254 | 1.9797 | 257.1558 | 2.1603 |
| | | 257.5254 | 1.9255 | 257.6558 | 2.2737 |
| | | 258.0254 | 2.0564 | 258.1558 | 2.2901 |
| | | 258.5254 | 2.0008 | 258.6558 | 2.2074 |
| | | 259.0254 | 1.9624 | 259.1558 | 2.2977 |
| | | 259.5254 | 2.0827 | 259.6558 | 2.2875 |
| | | 260.0254 | 2.0420 | 260.1558 | 2.2040 |
| | | 260.5254 | 1.9898 | 260.6558 | 2.3003 |
| | | 261.0254 | 2.1291 | 261.1558 | 2.2962 |
| | | 261.5254 | 2.0910 | 261.6558 | 2.2146 |
| | | 262.0254 | 2.0601 | 262.1558 | 2.3218 |
| | | 262.5254 | 2.1896 | 262.6558 | 2.2964 |
| | | 263.0254 | 2.1543 | 263.1558 | 2.1895 |
| | | 263.5254 | 2.1267 | 263.6558 | 2.3387 |
| | | 264.0254 | 2.2577 | 264.1558 | 2.3021 |
| | | 264.5254 | 2.2201 | 264.6558 | 2.2278 |
| | | 265.0254 | 2.1899 | 265.1558 | 2.3541 |
| | | 265.5254 | 2.3228 | 265.6558 | 2.3289 |
| | | 266.0254 | 2.2765 | 266.1558 | 2.2480 |
| | | 266.5254 | 2.2261 | 266.6558 | 2.3671 |
| | | 267.0254 | 2.3383 | 267.1558 | 2.3553 |
| | | 267.5254 | 2.2868 | 267.6558 | 2.2719 |
| | | 268.0254 | 2.2679 | 268.1558 | 2.3912 |
| | | 268.5254 | 2.3975 | 268.6558 | 2.3553 |
| | | 269.0254 | 2.3547 | 269.1558 | 2.2976 |
| | | 269.5254 | 2.3070 | 269.6558 | 2.4005 |
| | | 270.0254 | 2.4417 | 270.1558 | 2.3858 |
| | | 270.5254 | 2.3943 | 270.6558 | 2.3029 |
| | | 271.0254 | 2.3529 | 271.1558 | 2.4296 |
| | | 271.5254 | 2.4853 | 271.6558 | 2.4008 |
| | | 272.0254 | 2.4283 | 272.1558 | 2.3293 |
| | | 272.5254 | 2.3973 | 272.6558 | 2.4491 |
| | | 273.0254 | 2.5266 | 273.1558 | 2.4343 |
| | | 273.5254 | 2.4716 | 273.6558 | 2.3292 |
| | | 274.0254 | 2.4122 | 274.1558 | 2.4637 |
| | | 274.5254 | 2.5432 | 274.6558 | 2.4168 |
| | | 275.0254 | 2.5007 | 275.1558 | 2.3518 |
| | | 275.5254 | 2.4427 | 275.6558 | 2.4974 |
| | | 276.0254 | 2.5660 | 276.1558 | 2.4818 |
| | | 276.5254 | 2.4859 | 276.6558 | 2.3975 |
| | | 277.0254 | 2.4453 | 277.1558 | 2.4887 |
| | | 277.5254 | 2.5922 | 277.6558 | 2.4597 |
| | | 278.0254 | 2.5098 | 278.1558 | 2.4029 |
| | | 278.5254 | 2.4590 | 278.6558 | 2.4916 |
| | | 279.0254 | 2.5882 | 279.1558 | 2.4325 |
| | | 279.5254 | 2.5346 | 279.6558 | 2.3618 |
| | | 280.0254 | 2.4800 | 280.1558 | 2.4868 |
| | | 280.5254 | 2.5965 | 280.6558 | 2.4467 |
| | | 281.0254 | 2.5274 | 281.1558 | 2.3860 |
| | | 281.5254 | 2.4749 | 281.6558 | 2.5083 |
| | | 282.0254 | 2.5947 | 282.1558 | 2.4607 |
| | | 282.5254 | 2.5277 | 282.6558 | 2.3806 |
| | | 283.0254 | 2.4743 | 283.1558 | 2.5314 |
| | | 283.5254 | 2.6008 | 283.6558 | 2.4902 |
| | | 284.0254 | 2.5157 | 284.1558 | 2.4134 |
| | | 284.5254 | 2.4548 | 284.6558 | 2.5331 |
| | | 285.0254 | 2.5784 | 285.1558 | 2.5019 |
| | | 285.5254 | 2.4773 | 285.6558 | 2.4172 |
| | | 286.0254 | 2.4420 | 286.1558 | 2.5232 |
| | | 286.5254 | 2.5503 | 286.6558 | 2.5068 |
| | | 287.0254 | 2.4662 | 287.1558 | 2.4570 |
| | | 287.5254 | 2.4604 | 287.6558 | 2.5734 |
| | | 288.0254 | 2.5592 | 288.1558 | 2.5172 |
| | | 288.5254 | 2.4704 | 288.6558 | 2.4776 |
| | | 289.0254 | 2.4369 | 289.1558 | 2.5863 |
| | | 289.5254 | 2.5689 | 289.6558 | 2.5013 |
| | | 290.0254 | 2.4834 | 290.1558 | 2.4638 |
| | | 290.5254 | 2.4720 | 290.6558 | 2.5865 |
| | | 291.0254 | 2.5764 | 291.1558 | 2.5428 |
| | | 291.5254 | 2.4805 | 291.6558 | 2.5023 |
| | | 292.0254 | 2.4416 | 292.1558 | 2.6203 |
| | | 292.5254 | 2.5697 | 292.6558 | 2.5493 |
| | | 293.0254 | 2.5057 | 293.1558 | 2.4638 |
| | | 293.5254 | 2.5011 | 293.6558 | 2.5798 |
| | | 294.0254 | 2.6170 | 294.1558 | 2.5052 |
| | | 294.5254 | 2.5309 | 294.6558 | 2.0632 |
| | | 295.0254 | 2.5112 | 295.1558 | 2.1721 |
| | | 295.5254 | 2.6163 | 295.6558 | 2.0955 |
| | | 296.0254 | 2.5635 | 296.1558 | 2.0095 |
| | | 296.5254 | 2.5429 | 296.6558 | 2.0905 |
| | | 297.0254 | 2.6351 | 297.1558 | 2.0161 |
| | | 297.5254 | 2.5610 | 297.6558 | 1.9522 |
| | | 298.0254 | 2.5458 | 298.1558 | 2.0462 |
| | | 298.5254 | 2.6412 | 298.6558 | 1.9930 |
| | | 299.0254 | 2.5664 | 299.1558 | 1.9365 |
| | | 299.5254 | 2.5613 | 299.6558 | 2.0350 |
| | | 300.0254 | 2.6732 | 300.1558 | 1.9640 |
| | | 300.5254 | 2.5768 | 300.6558 | 1.7419 |
| | | 301.0254 | 2.5568 | 301.1558 | 1.7372 |
| | | 301.5254 | 2.6677 | 301.6558 | 1.5418 |
| | | 302.0254 | 2.5964 | 302.1558 | 0.9940 |
| | | 302.5254 | 2.5977 | 302.6558 | 1.1658 |
| | | 303.0254 | 2.6903 | 303.1558 | 0.9812 |
| | | 303.5254 | 2.6075 | 303.6558 | 0.7103 |
| | | 304.0254 | 2.6128 | 304.1558 | 0.1776 |
| | | 304.5254 | 2.7134 | 304.6558 | 0.1481 |
| | | 305.0254 | 2.6334 | 305.1558 | 0.1361 |
| | | 305.5254 | 2.6194 | 305.6558 | 0.2479 |
| | | 306.0254 | 2.7265 | 306.1558 | 0.1973 |
| | | 306.5254 | 2.6468 | 306.6558 | 0.1861 |
| | | 307.0254 | 2.6302 | 307.1558 | 0.2868 |
| | | 307.5254 | 2.7372 | 307.6558 | 0.2230 |
| | | 308.0254 | 2.6450 | 308.1558 | 0.1816 |
| | | 308.5254 | 2.6419 | 308.6558 | 0.2231 |
| | | 309.0254 | 2.7451 | 309.1558 | 0.1387 |
| | | 309.5254 | 2.6627 | 309.6558 | 0.0610 |
| | | 310.0254 | 2.6647 | 310.1558 | 0.0952 |
| | | 310.5254 | 2.7727 | 310.6558 | −0.0079 |
| | | 311.0254 | 2.6867 | 311.1558 | −0.0414 |
| | | 311.5254 | 2.6656 | 311.6558 | 0.0509 |
| | | 312.0254 | 2.7429 | 312.1558 | −0.0144 |
| | | 312.5254 | 2.6512 | 312.6558 | −0.0244 |
| | | 313.0254 | 2.6651 | 313.1558 | 0.0577 |
| | | 313.5254 | 2.7690 | 313.6558 | −0.0187 |
| | | 314.0254 | 2.6754 | 314.1558 | −0.0304 |
| | | 314.5254 | 2.6662 | 314.6558 | 0.0388 |
| | | 315.0254 | 2.7691 | 315.1558 | −0.0219 |
| | | 315.5254 | 2.6489 | 315.6558 | −0.0232 |
| | | 316.0254 | 2.6608 | 316.1558 | 0.0362 |
| | | 316.5254 | 2.7697 | 316.6558 | −0.0197 |
| | | 317.0254 | 2.6422 | | |
| | | 317.5254 | 2.6413 | | |
| | | 318.0254 | 2.7117 | | |
| | | 318.5254 | 2.6159 | | |
| | | 319.0254 | 2.6176 | | |
| | | 319.5254 | 2.7053 | | |
| | | 320.0254 | 2.6409 | | |
| | | 320.5254 | 2.5910 | | |
| | | 321.0254 | 2.6024 | | |
| | | 321.5254 | 2.4789 | | |
| | | 322.0254 | 2.4800 | | |
| | | 322.5254 | 2.5932 | | |
| | | 323.0254 | 2.5110 | | |
| | | 323.5254 | 2.5604 | | |
| | | 324.0254 | 2.6756 | | |
| | | 324.5254 | 2.5841 | | |
| | | 325.0254 | 2.6312 | | |
| | | 325.5254 | 2.7175 | | |
| | | 326.0254 | 2.6349 | | |
| | | 326.5254 | 2.5801 | | |
| | | 327.0254 | 2.5359 | | |
| | | 327.5254 | 2.3188 | | |
| | | 328.0254 | 2.0659 | | |
| | | 328.5254 | 1.8042 | | |
| | | 329.0254 | 1.6719 | | |
| | | 329.5254 | 1.1505 | | |
| | | 330.0254 | 0.9770 | | |
| | | 330.5254 | 0.5587 | | |
| | | 331.0254 | 0.5329 | | |
| | | 331.5254 | 0.3621 | | |
| | | 332.0254 | 0.2898 | | |
| | | 332.5254 | 0.3210 | | |
| | | 333.0254 | 0.3846 | | |
| | | 333.5254 | 0.3106 | | |
| | | 334.0254 | 0.3642 | | |
| | | 334.5254 | 0.4598 | | |
| | | 335.0254 | 0.2938 | | |
| | | 335.5254 | 0.2393 | | |
| | | 336.0254 | 0.3910 | | |
| | | 336.5254 | 0.3488 | | |
| | | 337.0254 | 0.0204 | | |
| | | 337.5254 | 0.0866 | | |
| | | 338.0254 | 0.0033 | | |
| | | 338.5254 | 0.0242 | | |
| | | 339.0254 | 0.0755 | | |
| | | 339.5254 | 0.0013 | | |
| | | 340.0254 | 0.0229 | | |
| | | 340.5254 | 0.0819 | | |
| | | 341.0254 | 0.0003 | | |
| | | 341.5254 | 0.0181 | | |
| | | 342.0254 | 0.0707 | | |
| | | 342.5254 | −0.0061 | | |
| | | 343.0254 | 0.0260 | | |
| | | 343.5254 | 0.0746 | | |
| | | 344.0254 | 0.0010 | | |
| | | 344.5254 | 0.0276 | | |
| | | 345.0254 | 0.0683 | | |

TABLE I-1
Data relating to Example 5, summarized in FIGS. 15 and 16

| | PLGA (rough) | | PLGA (smooth) | | SIS (rough) | | SIS (smooth) | | Dermabond Alone | | Native | |
|---|---|---|---|---|---|---|---|---|---|---|---|---|
| Aorta | T (N) | t (s) | T (N) | t (s) | T (N) | t (s) | T (N) | t (s) | T (N) | t (s) | T (N) | t (s) |
| 1 | 1.60 | 90 | 0.78 | 43 | 1.81 | 84 | 0.89 | 32 | 0.85 | 43 | 1.53 | 84 |
| 2 | 1.55 | 93 | 0.67 | 50 | 1.57 | 105 | 0.66 | 44 | 0.96 | 56 | 1.65 | 72 |
| 3 | 1.64 | 83 | 1.17 | 36 | 1.32 | 97 | 1.07 | 29 | 0.72 | 27 | 1.41 | 63 |
| 4 | 1.56 | 132 | 0.70 | 22 | 1.46 | 65 | 1.13 | 63 | 0.43 | 21 | 1.93 | 121 |
| 5 | 1.43 | 102 | 0.95 | 37 | 1.83 | 81 | 1.26 | 28 | 0.78 | 35 | 1.58 | 90 |
| 6 | 1.44 | 120 | 1.13 | 55 | 1.50 | 62 | 0.61 | 22 | 0.96 | 42 | 2.04 | 74 |
| 7 | 1.35 | 105 | 0.98 | 38 | 1.85 | 90 | 1.37 | 50 | 0.84 | 29 | 1.62 | 82 |
| 8 | 1.99 | 88 | 1.17 | 32 | 1.71 | 75 | 0.94 | 65 | 0.95 | 54 | 2.17 | 134 |
| 9 | 1.44 | 79 | 0.98 | 25 | 1.43 | 55 | 0.69 | 42 | 0.57 | 14 | 1.42 | 63 |
| 10 | 1.61 | 98 | 1.17 | 62 | 1.66 | 56 | 0.71 | 54 | 0.72 | 18 | 1.62 | 121 |
| Mean | 1.56 | 99 | 0.97 | 40 | 1.61 | 77 | 0.93 | 43 | 0.78 | 34 | 1.70 | 90 |
| St Dev | 0.18 | 17 | 0.20 | 13 | 0.19 | 17 | 0.27 | 15 | 0.18 | 15 | 0.26 | 26 |

TABLE I-2
Data relating to Example 5, summarized in FIGS. 15 and 16

| | PLGA (rough) | | PLGA (smooth) | | SIS (rough) | | SIS (smooth) | | Dermabond Alone | | Native | |
|---|---|---|---|---|---|---|---|---|---|---|---|---|
| Small Intestine | T (N) | t (s) | T (N) | t (s) | T (N) | t (s) | T (N) | t (s) | T (N) | t (s) | T (N) | t (s) |
| 1 | 0.96 | 55 | 0.85 | 35 | 1.25 | 22 | 0.80 | 52 | 0.54 | 29 | 1.16 | 94 |
| 2 | 1.21 | 82 | 0.60 | 70 | 1.10 | 61 | 0.86 | 41 | 0.24 | 10 | 1.42 | 82 |
| 3 | 0.79 | 67 | 0.55 | 50 | 0.72 | 50 | 0.69 | 22 | 0.77 | 32 | 1.36 | 93 |
| 4 | 0.95 | 33 | 0.85 | 60 | 1.41 | 73 | 0.55 | 25 | 0.52 | 12 | 1.11 | 45 |
| 5 | 1.22 | 48 | 0.50 | 45 | 1.25 | 50 | 0.67 | 18 | 0.58 | 15 | 0.58 | 76 |
| 6 | 1.29 | 81 | 0.45 | 5 | 1.08 | 72 | 0.46 | 12 | 0.21 | 8 | 1.24 | 89 |
| 7 | 0.87 | 75 | 0.55 | 45 | 0.87 | 55 | 0.77 | 41 | 0.83 | 36 | 0.68 | 77 |
| 8 | 0.88 | 71 | 0.40 | 5 | 1.14 | 40 | 0.62 | 15 | 0.38 | 14 | 1.24 | 56 |
| 9 | 1.21 | 45 | 0.95 | 50 | 1.30 | 35 | 0.93 | 32 | 0.16 | 6 | 0.77 | 86 |
| 10 | 0.80 | 66 | 1.04 | 55 | 0.70 | 65 | 0.50 | 8 | 0.48 | 24 | 1.30 | 39 |
| Mean | 1.02 | 62 | 0.67 | 42 | 1.08 | 52 | 0.69 | 27 | 0.47 | 19 | 1.09 | 74 |
| St Dev | 0.19 | 16 | 0.23 | 22 | 0.24 | 16 | 0.16 | 14 | 0.23 | 11 | 0.30 | 20 |

TABLE I-3
Data relating to Example 5, summarized in FIGS. 15 and 16

| | PLGA (rough) | | PLGA (smooth) | | SIS (rough) | | SIS (smooth) | | Dermabond Alone | | Native | |
|---|---|---|---|---|---|---|---|---|---|---|---|---|
| Liver | T (N) | t (s) | T (N) | t (s) | T (N) | t (s) | T (N) | t (s) | T (N) | t (s) | T (N) | t (s) |
| 1 | 1.26 | 52 | 1.01 | 36 | 1.41 | 37 | 1.06 | 38 | 0.85 | 35 | 1.24 | 70 |
| 2 | 1.28 | 64 | 1.08 | 50 | 1.34 | 52 | 1.08 | 32 | 0.53 | 18 | 1.27 | 54 |
| 3 | 1.25 | 60 | 0.94 | 44 | 1.08 | 23 | 1.03 | 35 | 0.65 | 32 | 1.14 | 44 |
| 4 | 1.23 | 47 | 1.03 | 58 | 1.11 | 47 | 0.95 | 27 | 1.04 | 39 | 1.45 | 64 |
| 5 | 1.42 | 42 | 1.15 | 24 | 1.12 | 32 | 0.82 | 20 | 0.47 | 13 | 1.48 | 85 |
| 6 | 1.10 | 38 | 1.19 | 35 | 1.07 | 45 | 0.88 | 33 | 0.59 | 30 | 1.42 | 43 |
| 7 | 1.17 | 42 | 1.00 | 22 | 0.92 | 12 | 0.90 | 36 | 0.82 | 47 | 1.30 | 68 |
| 8 | 1.22 | 58 | 1.18 | 32 | 1.44 | 57 | 0.99 | 42 | 0.52 | 32 | 1.28 | 37 |
| 9 | 1.30 | 55 | 1.25 | 46 | 1.25 | 63 | 0.75 | 25 | 0.56 | 36 | 1.21 | 47 |
| 10 | 1.42 | 74 | 0.86 | 18 | 1.16 | 37 | 1.02 | 57 | 0.41 | 22 | 1.43 | 72 |
| Mean | 1.27 | 53 | 1.07 | 37 | 1.19 | 41 | 0.95 | 35 | 0.64 | 30 | 1.32 | 58 |
| St Dev | 0.10 | 11 | 0.12 | 13 | 0.17 | 16 | 0.11 | 10 | 0.20 | 10 | 0.12 | 16 |

TABLE I-4
Data relating to Example 5, summarized in FIGS. 15 and 16

| | PLGA (rough) | | PLGA (smooth) | | SIS (rough) | | SIS (smooth) | | Dermabond Alone | | Native | |
|---|---|---|---|---|---|---|---|---|---|---|---|---|
| Spleen | T (N) | t (s) | T (N) | t (s) | T (N) | t (s) | T (N) | t (s) | T (N) | t (s) | T (N) | t (s) |
| 1 | 0.90 | 45 | 0.95 | 63 | 0.92 | 43 | 0.81 | 41 | 0.51 | 31 | 0.90 | 58 |
| 2 | 0.78 | 50 | 0.64 | 43 | 1.21 | 64 | 0.89 | 52 | 0.69 | 42 | 1.18 | 70 |
| 3 | 0.90 | 55 | 0.88 | 55 | 1.28 | 57 | 0.55 | 33 | 0.83 | 36 | 1.52 | 83 |
| 4 | 0.62 | 55 | 0.86 | 47 | 1.03 | 61 | 1.17 | 60 | 0.63 | 28 | 0.97 | 45 |
| 5 | 1.00 | 65 | 0.47 | 36 | 0.60 | 52 | 0.61 | 27 | 0.43 | 20 | 1.46 | 77 |
| 6 | 1.32 | 72 | 0.53 | 41 | 1.05 | 67 | 0.94 | 55 | 0.24 | 6 | 1.06 | 49 |
| 7 | 1.16 | 58 | 0.42 | 24 | 0.87 | 42 | 1.03 | 54 | 0.49 | 14 | 1.04 | 63 |
| 8 | 0.95 | 63 | 0.59 | 38 | 0.84 | 39 | 0.74 | 48 | 0.36 | 18 | 0.69 | 60 |
| 9 | 1.14 | 75 | 1.24 | 52 | 0.73 | 36 | 0.78 | 40 | 0.77 | 43 | 1.33 | 67 |
| 10 | 1.27 | 67 | 1.08 | 49 | 0.95 | 55 | 0.65 | 29 | 0.27 | 8 | 0.84 | 41 |
| Mean | 1.00 | 61 | 0.77 | 45 | 0.95 | 52 | 0.82 | 44 | 0.52 | 25 | 1.10 | 61 |
| St Dev | 0.22 | 10 | 0.28 | 11 | 0.21 | 11 | 0.19 | 12 | 0.20 | 13 | 0.27 | 14 |

TABLE I-5
Data relating to Example 5, summarized in FIGS. 15 and 16

| | PLGA (rough) | | PLGA (smooth) | | SIS (rough) | | SIS (smooth) | | Dermabond Alone | | Native | |
|---|---|---|---|---|---|---|---|---|---|---|---|---|
| Lung | T (N) | t (s) | T (N) | t (s) | T (N) | t (s) | T (N) | t (s) | T (N) | t (s) | T (N) | t (s) |
| 1 | 0.46 | 34 | 0.32 | 26 | 0.46 | 43 | 0.30 | 27 | 0.20 | 22 | 0.57 | 43 |
| 2 | 0.32 | 22 | 0.40 | 42 | 0.35 | 36 | 0.37 | 36 | 0.00 | 1 | 0.66 | 52 |
| 3 | 0.38 | 26 | 0.36 | 32 | 0.42 | 45 | 0.28 | 15 | 0.09 | 6 | 0.56 | 38 |
| 4 | 0.58 | 48 | 0.29 | 27 | 0.40 | 43 | 0.44 | 32 | 0.28 | 15 | 0.65 | 48 |
| 5 | 0.51 | 45 | 0.31 | 24 | 0.29 | 25 | 0.30 | 24 | 0.34 | 28 | 0.68 | 45 |
| 6 | 0.40 | 31 | 0.48 | 38 | 0.36 | 41 | 0.26 | 26 | 0.18 | 16 | 0.63 | 36 |
| 7 | 0.36 | 39 | 0.28 | 26 | 0.32 | 36 | 0.45 | 36 | 0.22 | 21 | 0.54 | 46 |
| 8 | 0.63 | 52 | 0.32 | 28 | 0.48 | 46 | 0.28 | 24 | 0.29 | 19 | 0.43 | 32 |
| 9 | 0.55 | 48 | 0.19 | 20 | 0.54 | 45 | 0.33 | 29 | 0.31 | 24 | 0.51 | 46 |
| 10 | 0.50 | 42 | 0.24 | 22 | 0.44 | 40 | 0.47 | 35 | 0.21 | 18 | 0.72 | 52 |
| Mean | 0.47 | 39 | 0.32 | 29 | 0.41 | 40 | 0.35 | 28 | 0.21 | 17 | 0.60 | 44 |
| St Dev | 0.10 | 10 | 0.08 | 7 | 0.08 | 6 | 0.08 | 7 | 0.10 | 8 | 0.09 | 7 |

TABLE J-1
Data relating to Example 6, summarized in FIGS. 17 and 18

| | 10 mm × | | 10 mm × | | 5 mm × | | 5 mm × | | 15 mm × | | 15 mm × | |
|---|---|---|---|---|---|---|---|---|---|---|---|---|
| | 10 mm | | 5 mm | | 10 mm | | 5 mm | | 10 mm | | 5 mm | |
| Aorta | T (N) | t (s) | T (N) | t (s) | T (N) | t (s) | T (N) | t (s) | T (N) | t (s) | T (N) | t (s) |
| 1 | 1.60 | 90 | 1.30 | 94 | 1.22 | 103 | 0.76 | 32 | 2.02 | 132 | 1.44 | 83 |
| 2 | 1.55 | 93 | 0.86 | 87 | 1.36 | 96 | 0.55 | 40 | 1.88 | 89 | 1.32 | 72 |
| 3 | 1.64 | 83 | 0.77 | 92 | 1.27 | 93 | 0.71 | 25 | 1.93 | 94 | 1.39 | 104 |
| 4 | 1.56 | 132 | 0.93 | 77 | 0.75 | 67 | 0.35 | 15 | 2.10 | 106 | 1.50 | 121 |
| 5 | 1.43 | 102 | 0.74 | 45 | 0.96 | 75 | 0.39 | 12 | 2.24 | 156 | 1.17 | 90 |
| 6 | 1.44 | 120 | 0.77 | 56 | 0.84 | 54 | 0.57 | 27 | 1.74 | 80 | 1.37 | 96 |
| 7 | 1.35 | 105 | 1.17 | 82 | 1.07 | 66 | 0.83 | 48 | 2.32 | 141 | 1.32 | 85 |
| 8 | 1.99 | 88 | 1.09 | 85 | 1.14 | 99 | 0.64 | 33 | 2.16 | 120 | 1.45 | 112 |
| 9 | 1.44 | 79 | 0.90 | 64 | 0.88 | 71 | 0.59 | 38 | 1.96 | 102 | 1.48 | 108 |
| 10 | 1.61 | 98 | 0.98 | 80 | 0.79 | 62 | 0.79 | 21 | 2.15 | 98 | 1.53 | 87 |
| Mean | 1.56 | 99 | 0.95 | 76 | 1.03 | 79 | 0.62 | 29 | 2.05 | 112 | 1.40 | 96 |
| St Dev | 0.18 | 17 | 0.19 | 16 | 0.21 | 18 | 0.16 | 11 | 0.18 | 25 | 0.11 | 15 |

TABLE J-2
Data relating to Example 6, summarized in FIGS. 17 and 18

| | 10 mm × | | 10 mm × | | 5 mm × | | 5 mm × | | 15 mm × | | 15 mm × | |
|---|---|---|---|---|---|---|---|---|---|---|---|---|
| Small | 10 mm | | 5 mm | | 10 mm | | 5 mm | | 10 mm | | 5 mm | |
| Intestine | T (N) | t (s) | T (N) | t (s) | T (N) | t (s) | T (N) | t (s) | T (N) | t (s) | T (N) | t (s) |
| 1 | 0.96 | 55 | 0.80 | 74 | 0.67 | 55 | 0.34 | 15 | 1.21 | 99 | 1.17 | 78 |
| 2 | 1.21 | 82 | 0.92 | 69 | 0.82 | 83 | 0.27 | 20 | 1.27 | 117 | 0.94 | 61 |
| 3 | 0.79 | 67 | 0.63 | 34 | 0.71 | 61 | 0.48 | 35 | 1.36 | 92 | 0.82 | 65 |
| 4 | 0.95 | 33 | 0.74 | 53 | 0.94 | 69 | 0.51 | 44 | 1.47 | 111 | 0.88 | 73 |
| 5 | 1.22 | 48 | 0.55 | 51 | 0.54 | 42 | 0.22 | 24 | 1.33 | 96 | 0.73 | 56 |
| 6 | 1.29 | 81 | 0.60 | 44 | 0.60 | 49 | 0.28 | 27 | 1.36 | 103 | 0.80 | 79 |
| 7 | 0.87 | 75 | 0.52 | 41 | 0.63 | 61 | 0.43 | 39 | 1.39 | 104 | 1.00 | 85 |
| 8 | 0.88 | 71 | 0.46 | 32 | 0.57 | 58 | 0.36 | 32 | 1.90 | 151 | 0.92 | 81 |
| 9 | 1.21 | 45 | 0.58 | 66 | 0.51 | 40 | 0.18 | 8 | 1.52 | 125 | 0.62 | 71 |
| 10 | 0.80 | 66 | 0.64 | 56 | 0.59 | 53 | 0.41 | 32 | 1.42 | 88 | 0.90 | 93 |
| Mean | 1.02 | 62 | 0.64 | 52 | 0.66 | 57 | 0.35 | 28 | 1.42 | 109 | 0.88 | 74 |
| St Dev | 0.19 | 16 | 0.14 | 15 | 0.13 | 13 | 0.11 | 11 | 0.19 | 19 | 0.15 | 11 |

TABLE K-1
Data relating to Example 7, summarized in FIGS. 20 and 21

| | Sandpaper | | Computer-Drilling | | Punch | | Mold | |
|---|---|---|---|---|---|---|---|---|
| Aorta | T (N) | t (s) | T (N) | t (s) | T (N) | t (s) | T (N) | t (s) |
| 1 | 1.47 | 78 | 1.04 | 84 | 1.59 | 132 | 1.42 | 113 |
| 2 | 1.82 | 118 | 0.83 | 79 | 1.63 | 126 | 0.98 | 72 |
| 3 | 1.55 | 97 | 0.99 | 93 | 1.92 | 137 | 1.06 | 94 |
| 4 | 1.57 | 125 | 1.15 | 112 | 1.47 | 96 | 1.36 | 132 |
| 5 | 1.32 | 69 | 0.87 | 66 | 1.33 | 100 | 1.25 | 99 |
| Mean | 1.55 | 97 | 0.98 | 87 | 1.59 | 118 | 1.21 | 102 |
| St Dev | 0.18 | 24 | 0.13 | 17 | 0.22 | 19 | 0.19 | 22 |

TABLE K-2
Data relating to Example 7, summarized in FIGS. 20 and 21

| | | | Computer- | | | | | |
|---|---|---|---|---|---|---|---|---|
| Small | Sandpaper | | Drilling | | Punch | | Mold | |
| Intestine | T (N) | t (s) | T (N) | t (s) | T (N) | t (s) | T (N) | t (s) |
| 1 | 1.07 | 60 | 0.47 | 40 | 1.00 | 72 | 0.75 | 64 |
| 2 | 0.94 | 48 | 0.72 | 62 | 1.03 | 69 | 0.63 | 44 |
| 3 | 1.00 | 74 | 0.55 | 44 | 1.26 | 83 | 0.81 | 77 |
| 4 | 1.23 | 89 | 0.69 | 65 | 1.32 | 81 | 0.77 | 59 |
| 5 | 0.92 | 44 | 0.43 | 45 | 0.75 | 53 | 0.90 | 72 |
| Mean | 1.03 | 63 | 0.57 | 51 | 1.07 | 72 | 0.77 | 63 |
| St Dev | 0.13 | 19 | 0.13 | 11 | 0.23 | 12 | 0.10 | 13 |

TABLE K-3
Data relating to Example 7, summarized in FIGS. 20 and 21

| | | | Computer- | | | | | |
|---|---|---|---|---|---|---|---|---|
| | Sandpaper | | Drilling | | Punch | | Mold | |
| Liver | T (N) | t (s) | T (N) | t (s) | T (N) | t (s) | T (N) | t (s) |
| 1 | 1.32 | 57 | 1.26 | 55 | 1.42 | 57 | 0.92 | 55 |
| 2 | 1.46 | 55 | 1.19 | 61 | 1.29 | 53 | 1.01 | 58 |
| 3 | 1.10 | 39 | 0.82 | 52 | 1.11 | 49 | 1.06 | 67 |
| 4 | 1.29 | 47 | 0.37 | 23 | 1.53 | 66 | 0.82 | 43 |
| 5 | 1.33 | 67 | 0.55 | 46 | 1.58 | 68 | 0.90 | 48 |
| Mean | 1.30 | 53 | 0.84 | 47 | 1.39 | 59 | 0.94 | 54 |
| St Dev | 0.13 | 11 | 0.39 | 15 | 0.19 | 8 | 0.09 | 9 |

TABLE K-4
Data relating to Example 7, summarized in FIGS. 20 and 21

| | | | Computer- | | | | | |
|---|---|---|---|---|---|---|---|---|
| | Sandpaper | | Drilling | | Punch | | Mold | |
| Spleen | T (N) | t (s) | T (N) | t (s) | T (N) | t (s) | T (N) | t (s) |
| 1 | 0.99 | 60 | 0.72 | 61 | 1.01 | 55 | 0.78 | 55 |
| 2 | 0.82 | 49 | 0.64 | 58 | 0.97 | 49 | 0.53 | 60 |
| 3 | 0.90 | 53 | 0.57 | 40 | 1.15 | 62 | 0.66 | 63 |
| 4 | 1.04 | 62 | 0.81 | 73 | 1.32 | 76 | 0.87 | 66 |
| 5 | 1.25 | 74 | 0.6 | 44 | 1.09 | 67 | 0.99 | 71 |
| Mean | 1.00 | 60 | 0.67 | 55 | 1.11 | 62 | 0.77 | 63 |
| St Dev | 0.16 | 10 | 0.10 | 13 | 0.14 | 10 | 0.18 | 6 |

TABLE K-5
Data relating to Example 7, summarized in FIGS. 20 and 21

| | | | Computer- | | | | | |
|---|---|---|---|---|---|---|---|---|
| | Sandpaper | | Drilling | | Punch | | Mold | |
| Lung | T (N) | t (s) | T (N) | t (s) | T (N) | t (s) | T (N) | t (s) |
| 1 | 0.48 | 36 | 0.29 | 27 | 0.62 | 48 | 0.29 | 31 |
| 2 | 0.75 | 46 | 0.38 | 35 | 0.51 | 51 | 0.41 | 42 |
| 3 | 0.43 | 32 | 0.43 | 38 | 0.43 | 37 | 0.33 | 37 |
| 4 | 0.50 | 43 | 0.23 | 31 | 0.47 | 32 | 0.38 | 46 |
| 5 | 0.37 | 31 | 0.25 | 30 | 0.60 | 45 | 0.44 | 39 |
| Mean | 0.51 | 38 | 0.32 | 32 | 0.53 | 43 | 0.37 | 39 |
| St Dev | 0.15 | 7 | 0.09 | 4 | 0.08 | 8 | 0.06 | 6 |

1. A composition suitable for medical and surgical applications, comprising:
a scaffold including at least one of a biological material, biocompatible material, and biodegradable material, and
a non-light activated adhesive including at least one of a biological material, biocompatible material, and biodegradable material, coupled to the scaffold to form a composite that, when used to repair biological tissue, has a tensile strength of at least about 120% of the tensile strength of the adhesive alone.

2. The composition of claim 1, wherein the scaffold is selected from the group consisting of poly(glycolic acid), poly (L-lactic-co-glycolic acid,) poly (epsilon-caprolactoma), poly(ethyleneglycol), poly (alpha ester)s, poly (ortho ester)s, poly (anhydride)s, small intestine submucosa, polymerized collagen, polymerized elastin.

3. The composition of claim 1, wherein the adhesive is selected from the group consisting of serum albumin, collagen, fibrin, fibrinogen, fibronectin, thrombin, barnacle glues, marine algae, cyanoacrylates.

4. The composition of claim 1, wherein the scaffold has an, at least partially, irregular surface.

5. The composition of claim 1, wherein the scaffold has a pore size in the range of about 100-500 μm.

6. The composition of claim 1, further comprising an activator.

7. The composition of claim 1, further comprising a dopant.

8. The composition of claim 1, wherein the composite, when used to repair biological tissue, exhibits a substantially constant tensile strength in response to a substantially constant application of force for a period at least about 130% longer than the adhesive alone.

9. The composition of claim 1, wherein the scaffold has a surface area, and the scaffold is selected for a medical or surgical application based on the surface area.

10. A method for repairing, joining, aligning, or sealing biological tissue, comprising the steps of:
combining a biological, biocompatible, or biodegradable scaffold and a non-light activated biological, biocompatible, or biodegradable adhesive to form a composition having a tensile strength of at least about 120% of the tensile strength of the adhesive alone, and
applying the composite to an adhesion site.

11. The method of claim 10, further comprising the step of combining an activator with the composite.

12. The method of claim 11, wherein the step of combining an activator with the composite is performed prior to the applying step.

13. The method of claim 10, further comprising the step of combining a dopant with the composite.

14. The method of claim 13, wherein the step of combining a dopant with the composite is performed prior to the applying step.

15. The method of claim 10, wherein the adhesion site is a portion of biological tissue.

16. The method of claim 10, wherein the adhesion site is a portion of a biocompatible implant.

17. The method of claim 10 wherein the applying step is performed as part of an internal surgical procedure.

18. The method of claim 10, wherein the applying step is performed as part of an external surgical procedure.

19. The method of claim 10, wherein the applying step is performed during an emergency medical procedure.

20. The method of claim 10, wherein the applying step includes the step of placing the composite over edges of severed tissue.

21. A product for joining, repairing, aligning or sealing biological tissue, comprising:
a biological, biocompatible, or biodegradable scaffold,
a biological, biocompatible, or biodegradable non-light activated adhesive, and
a device that facilitates combination of the scaffold and the adhesive to form a composite having a tensile strength of at least about 120% of the tensile strength of the adhesive alone.

22. The product of claim 21, further comprising instructions for coupling the scaffold and the adhesive.

23. The product of claim 21, further comprising an applicator suitable to apply the composite to an adhesion site.

24. The product of claim 21, further comprising instructions for applying the composite to an adhesion site.

25. The product of claim 21, further comprising an inert, removable material covering the scaffold and adhesive.

26. The product of claim 21, further comprising a fracturable membrane coupled to the adhesive.

27. The product of claim 21, further comprising a separator positioned between the scaffold and the adhesive.

28. The product of claim 27, further comprising a grip coupled to the separator such that exertion of force on the grip removes the separator from between the scaffold and the adhesive.

29. The product of claim 21, further comprising an activator and a first separator positioned between the activator and the adhesive.

30. The product of claim 29, further comprising a grip coupled to the separator such that exertion of a force on the grip causes the separator to be removed from between the activator and the adhesive.

31. The product of claim 29, further comprising a second separator positioned between the scaffold and the adhesive.

32. The product of claim 31, further comprising a grip coupled to the first separator and the second separator such that exertion of a force on the grip causes the first and second separators to be removed.

33. The product of claim 31, further comprising a first grip coupled to the first separator and a second grip coupled to the second separator.

34. The product of claim 31, further comprising a grip coupled to the second separator such that exertion of a force on the grip causes the second separator to be removed from between the scaffold and the adhesive.
